(19) United States
(12) Patent Application Publication
(10) Pub. No.: US 2005/0277175 A1
(43) Pub. Date: Dec. 15, 2005

(54) Truncated ADAMTS molecules

(75) Inventors: Edward R. LaVallie, Harvard, MA (US); Lisa A. Collins-Racie, Acton, MA (US); Christopher John Corcoran, Arlington, MA (US); Natalie C. Twine, Goffstown, NH (US); Michael J. Agostino, Andover, MA (US)

Correspondence Address: FITZPATRICK CELLA (WYETH), 30 ROCKEFELLER PLAZA, NEW YORK, NY, 10112-3800, US

(73) Assignee: WYETH, Madison, NJ (US)

(21) Appl. No.: 11/109,157

(22) Filed: Apr. 18, 2005

Related U.S. Application Data
(60) Provisional application No. 60/562,685, filed on Apr. 16, 2004.

(51) Int. Cl. C12P 21/06; C12N 9/64; C07H 21/04

(52) U.S. Cl. 435/69.1; 435/226; 435/320.1; 435/325; 536/23.2

(57) ABSTRACT

The invention provides truncated biologically active ADAMTS polypeptides, particularly those with hyalectenase activity, and more particularly those with aggrecanase activity, that exhibit greater stability and homogeneity and higher expression yields than their full-length counterparts. The invention also provides nucleic acid molecules encoding such truncated biologically active ADAMTS polypeptides and methods for producing the truncated biologically active ADAMTS polypeptides. In addition, the invention provides methods for identifying compounds capable of modulating biologically active ADAMTS polypeptides, particularly those compounds that inhibit aggrecanase activity.

This application claims the benefit of U.S. Provisional Patent Application No. 60/562,685, filed Apr. 16, 2004.

### BACKGROUND OF THE INVENTION

1. Field of the Invention

The present invention relates to novel truncated ADAMTS polypeptides, particularly those with aggrecanase activity, as well as nucleic acid molecules encoding such novel polypeptides. The invention further relates to methods for producing such novel truncated ADAMTS polypeptides, as wells as methods employing such novel polypeptides to develop ADAMTS inhibitors, particularly aggrecanase inhibitors.

2. Related Background Art

ADAM (“a disintegrin and metalloproteinase”) proteins represent a family of membrane-associated multidomain zinc-dependent metalloproteases with high sequence homology and domain organization. In addition to the disintegrin and protease domains, the ADAM proteins generally contain a prodomain, a cysteine-rich domain, an EGF-like domain, a transmembrane domain, and a cytoplasmic tail domain. The ADAM proteins are unique among cell-surface proteins in containing features of both adhesive proteins and proteases (Kaushal and Shah, J. Clin. Invest. 105:1335 (2000)).

Recently, new members of the ADAM family have been identified which lack the transmembrane and cytoplasmic tail domains. More importantly, these new members contain unique thrombospondin type I repeats (TSRs) not found in other ADAMs. These ADAMTS (“a disintegrin and metalloproteinase with thrombospondin motifs”) proteins also contain a prodomain, a metalloprotease domain, a disintegrin domain, a cysteine-rich domain, and a spacer region, and may also contain a PLAC domain which is a 30-40 amino acid peptide containing six cysteins. Like other ADAM proteins, all ADAMTS proteins identified to date contain the catalytic consensus sequence HXXGXXHD, which coordinates the Zn²⁺ ion necessary for protease activity (Tang, Int. J. Biochem. Cell Biol. 445:223 (2001)).

The members of the ADAMTS family, which now number over twenty, all contain a single TSR following the disintegrin domain; this internal TSR has been shown to bind to heparin (Kuno et al., J. Biol. Chem. 272:556 (1997)). The ADAMTSs, however, can be distinguished from each other, in part, by the variable number of C-terminal TSRs they contain downstream of the spacer region. For example, ADAMTS-4 contains no C-terminal TSRs, ADAMTS-5 contains one C-terminal TSR, ADAMTS-1 (as well as the human homolog METH1) and ADAMTS-16 contain two C-terminal TSRs, ADAMTS-10 and -18 contain five C-terminal TSRs, and ADAMTS-9 and -20 contain fourteen C-terminal TSRs.

The ADAMTSs have been implicated in a variety of pathological disorders. For example, mutations in ADAMTS-2 result in Ehlers-Danlos syndrome in humans and dermatosparaxis in cattle (Colige et al., Am. J. Hum. Genet. 65:308 (1999)), while mutations in ADAMTS-13 (also known as the von Willebrand factor cleaving protein) result in thrombotic thrombocytopenic purpura (Kokame et al., Proc. Natl. Acad. Sci. USA 99:11902 (2002)).

Recently, several ADAMTSs have also been implicated in the pathophysiological events leading to inflammatory disorders of articular cartilage, such as osteoarthritis (OA) and rheumatoid arthritis (RA). ADAMTS-4 and ADAMTS-5 (the latter also known as ADAMTS-11) were originally identified as the proteases responsible for the cleavage of aggrecan (they are now termed aggrecanase-1 and aggrecanase-2, respectively), which contributes to the mechanical properties of articular cartilage in withstanding compressive deformation under load (Tortorella et al., Science 284:1664 (1999); Abbaszade et al., J. Biol. Chem. 274:23443 (1999)). Subsequently, ADAMTS-1 was also shown to possess this cartilage-damaging “aggrecanase” activity (Rodriguez-Manzaneque et al., Biochem. Biophys. Res. Commun. 293:501 (2002)). There is also evidence to suggest that these aggrecanases possess brain-enriched hyaluronan binding/brevican cleavage activity, which may play a role in the invasiveness of gliomas (Matthews et al., J. Biol. Chem. 275:22695 (2000)). Aggrecanases are more generally referred to as hyalectanases because they cleave hyalectans, which include aggrecan, brevican and versican.

The ADAMTS aggrecanases cleave between amino acids Glu³⁷³-Ala³⁷⁴ within the interglobular domain of the G1 globular domain of aggrecan, which exposes an N-terminal neoepitope (³⁷⁴ARGSV) on the resulting C-terminal aggrecan fragment (Tortorella et al., Matrix Biol. 21:499 (2002); Westling et al., J. Biol. Chem. 277:16059 (2002); Tortorella et al., J. Biol. Chem. 275:18566 (2000)). This ³⁷⁴ARGSV aggrecan fragment has been found in synovial fluid from patients with inflammatory joint disease, joint injury, and OA (Malfait et al., J. Biol. Chem. 277:22201 (2002); Lohmander et al., Arthritis Rheum. 36:1214 (1993); Sandy et al., J. Clin. Invest. 89:1512 (1992)). In addition, the resulting N-terminal aggrecan fragment containing the C-terminal NITEGE³⁷³ neoepitope has been found in articular cartilage from patients with joint injury, OA, and RA (Malfait et al., supra; Sandy and Verscharen, Biochem. J. 358:615 (2001); Lark et al., J. Clin. Invest. 100:93 (1997)). Ihibition of aggrecanase activity with a synthetic ADAMTS inhibitor has been shown to prevent aggrecan degradation in osteoarthritic cartilage, as measured by release of aggrecan fragments containing the ³⁷⁴ARGSV neoepitope (Malfait et al., supra).

Because of their involvement in various inflammatory disorders such as OA and RA, there is a need to identify inhibitors of the ADAMTS aggrecanases, particularly small molecule inhibitors. To do so, large amounts of purified, homogeneous aggrecanase proteins are required to perform the necessary screening assays and crystallization studies. It has proved difficult, however, to isolate and purify large amounts of these proteins due to the heterogeneity, low expression, and poor stability of these molecules. For example, recombinant expression of aggrecanase-1 (ADAMTS-4) yields several isoforms with molecular weights lower than the mature protein due to C-terminal truncations at various positions in the polypeptide (Flannery et al., J. Biol. Chem. 277:42775 (2002); Gao et al., J. Biol. Chem. 277:11034 (2002)). In addition, native aggrecanase-1 and -2 (ADAMTS-5) both exist in various low molecular weight forms indicative of C-terminal truncation (Tortorella et al., J. Biol. Chem. 275:25791 (2000); Abbaszade, supra).

United States Patent Application Publication No. 2004/0044194 A1, incorporated herein in its entirety by reference, relates to ADAMTS 18 nucleic acid molecules and polypeptides encoded thereby.

United States Patent Application Publication No. 2004/0054149 A1, incorporated herein in its entirety by reference, relates to truncated ADAMTS molecules and preferably truncated ADAMTS-4 (aggrecanase-1) and ADAMTS-5 (aggrecanase-2) nucleic acid molecules and polypeptides encoded thereby. Simularly, U.S. Patent Application Publication No. 2004/0142863 A1, incorporated herein in its entirety by reference, relates to truncated ADAMTS-4 nucleic acid molecules and polypeptides encoded thereby.

The truncated ADAMTS molecules described heretofore are generally truncated at the c-terminus. There still exists a need to identify other ADAMTS-related molecules, and particularly truncated ADAMTS molecules that are useful to increase the yield, stability, and homogeneity of ADAMTS aggrecanases.

### SUMMARY OF THE INVENTION

The invention provides truncated biologically active ADAMTS polypeptides, particularly those with hyalectenase activity, and more particularly those with aggrecanase activity, that exhibit greater stability and homogeneity and higher expression yields than their full-length counterparts. In one aspect, the truncated ADAMTS lacks a substantial portion of the cysteine-rich domain. Preferably the truncated ADAMTS retains a substantial portion of the catalytic domain, disintegrin domain, and the central thrombospondin type 1 repeat. In a particular embodiment, the truncated ADAMTS polypeptides lack a substantial portion of the c-terminus after the conserved Phe, and may further lack, or alternatively lack the prodomain. The invention also provides nucleic acid molecules encoding such truncated biologically active ADAMTS polypeptides. The invention further provides methods for producing such truncated biologically active ADAMTS polypeptides, as well as methods for identifying compounds capable of modulating biologically active ADAMTS polypeptides, particularly those compounds that inhibit aggrecanase activity.

In one aspect of the invention, there is provided an isolated or recombinant aggrecanase obtainable by deleting from a full-length ADAMTS protein a plurality of amino acid residues, wherein the full-length ADAMTS protein comprises a cysteine-rich domain, and the plurality of deleted amino acid residues comprise a substantial portion of the cysteine-rich domain, and wherein the full-length ADAMTS protein is not a full-length ADAMTS-4 protein.

### BRIEF DESCRIPTION OF THE DRAWINGS

FIG. 1 schematically depicts the domain structures of ADAMTS-7, -9, -10, -16, and -18 proteins.

FIG. 2 schematically illustrates the domain structures of modified ADAMTS-7, -9, -10, -16, and -18 proteins.

FIG. 3 shows the amino acid sequence of (a) modified ADAMTS-7 lacking the prodomain (SEQ ID NO:2); (b) modified ADAMTS-7 lacking the c-terminus after the conserved Phe (SEQ ID NO:3); and (c) modified ADAMTS-7 lacking both the prodomain and the c-terminus after the conserved Phe (SEQ ID NO:4).

FIG. 4 shows the amino acid sequence of (a) modified ADAMTS-9 lacking the prodomain (SEQ ID NO:6); (b) modified ADAMTS-9 lacking the c-terminus after the conserved Phe (SEQ ID NO:7); and (c) modified ADAMTS-9 lacking both the prodomain and the c-terminus after the conserved Phe (SEQ ID NO:8).

FIG. 5 shows the amino acid sequence of (a) modified ADAMTS-10 lacking the prodomain (SEQ ID NO:10); (b) modified ADAMTS-10 lacking the c-terminus after the conserved Phe (SEQ ID NO:11); and (c) modified ADAMTS-10 lacking both the prodomain and the c-terminus after the conserved Phe (SEQ ID NO:12).

FIG. 6 shows the amino acid sequence of (a) modified ADAMTS-16 lacking the prodomain (SEQ ID NO:14); (b) modified ADAMTS-16 lacking the c-terminus after the conserved Phe (SEQ ID NO:15); and (c) modified ADAMTS-16 lacking both the prodomain and the c-terminus after the conserved Phe (SEQ ID NO:16).

FIG. 7 shows the amino acid sequence of (a) modified ADAMTS-18 lacking the prodomain (SEQ ID NO:18); (b) modified ADAMTS-18 lacking the c-terminus after the conserved Phe (SEQ ID NO:19); and (c) modified ADAMTS-18 lacking both the prodomain and the c-terminus after the conserved Phe (SEQ ID NO:20).

FIG. 8 shows a Western blot of the neoepitope-containing aggrecan G1 domain following incubation of bovine aggrecan with (a) truncated ADAMTS-7; (b) truncated ADAMTS-9; (c) truncated ADAMTS-10; (d) truncated ADAMTS-16; and (e) truncated ADAMTS-18.

All drawings are included for illustration, and should not be construed as limiting the present invention.

### DETAILED DESCRIPTION OF THE INVENTION

The present invention is based on the discovery that truncated forms of ADAMTS proteins have greater stability and higher expression levels and are more homogenous than their full-length counterparts, while still retaining biological activity. As such, the present invention provides novel truncated forms of biologically active ADAMTS proteins, particularly those with hyalectanase activity and more particularly those with aggrecanase activity, that possess greater stability and higher expression levels than the full-length forms of the proteins.

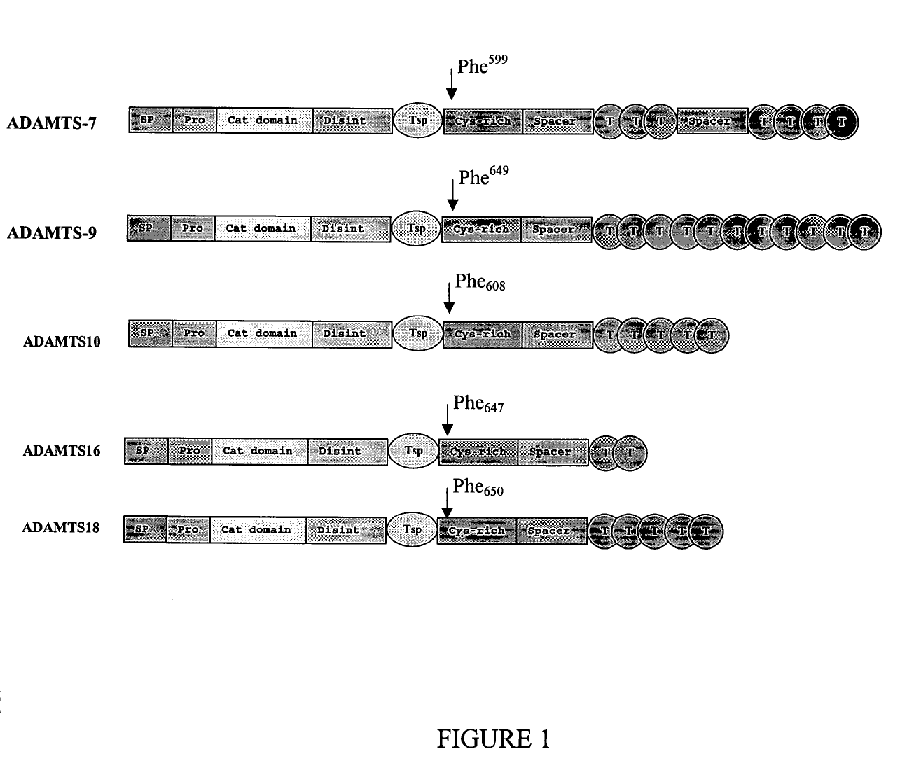

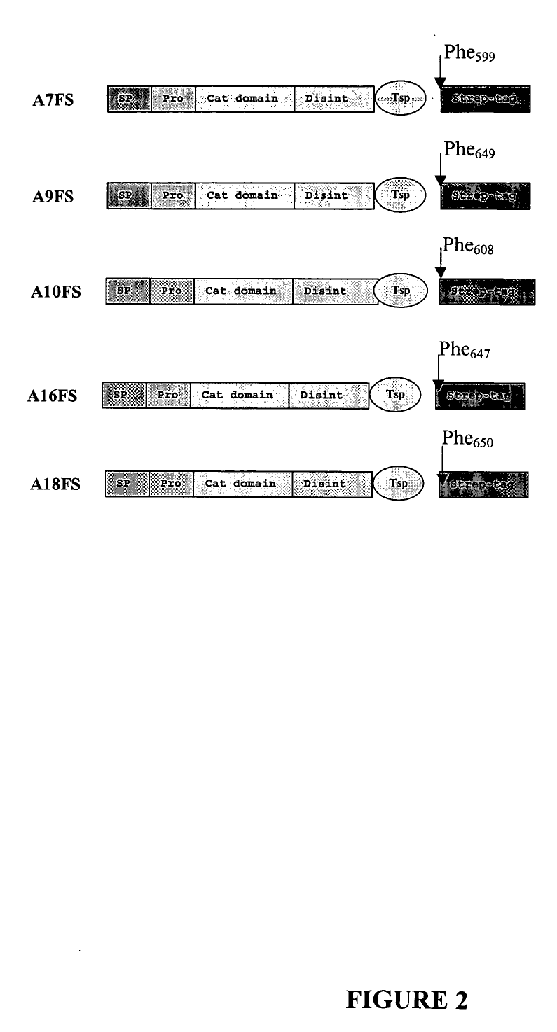

In a preferred embodiment, the truncated ADAMTS molecules are truncated at the c-terminus and retain hyalectanase activity, and preferably aggrecanase activity. In another preferred embodiment, the truncated ADAMTS molecules comprise a substantial truncation of the c-terminus after the conserved phenylalanine (Phe) shown in FIGS. 1 and 2. In yet another preferred embodiment, the truncated ADAMTS molecules lack a substantial portion of the prodomain and retain hyalectanase activity, and preferably aggrecanase activity. In a particularly preferred embodiment, a substantial portion of the cysteine rich domain is deleted, such that the truncated ADAMTS retains hyalectanase activity, and more preferably aggrecanase activity.

In one aspect of the invention, a truncated ADAMTS with hyalectanase activity, and more preferably with aggrecanase activity, is a truncated ADAMTS lacking at least the prodomain. Such truncated ADAMTS molecules include, inter alia, ADAMTS-4, ADAMTS-5, ADAMTS-7, ADAMTS-9, ADAMTS-10, ADAMTS-16 and ADAMTS-18 lacking at least the prodomain. These truncated ADAMTS molecules having hyalectanase acitivity may further comprise a c-terminal truncation, for example, a truncation at the c-terminal conserved Phe.

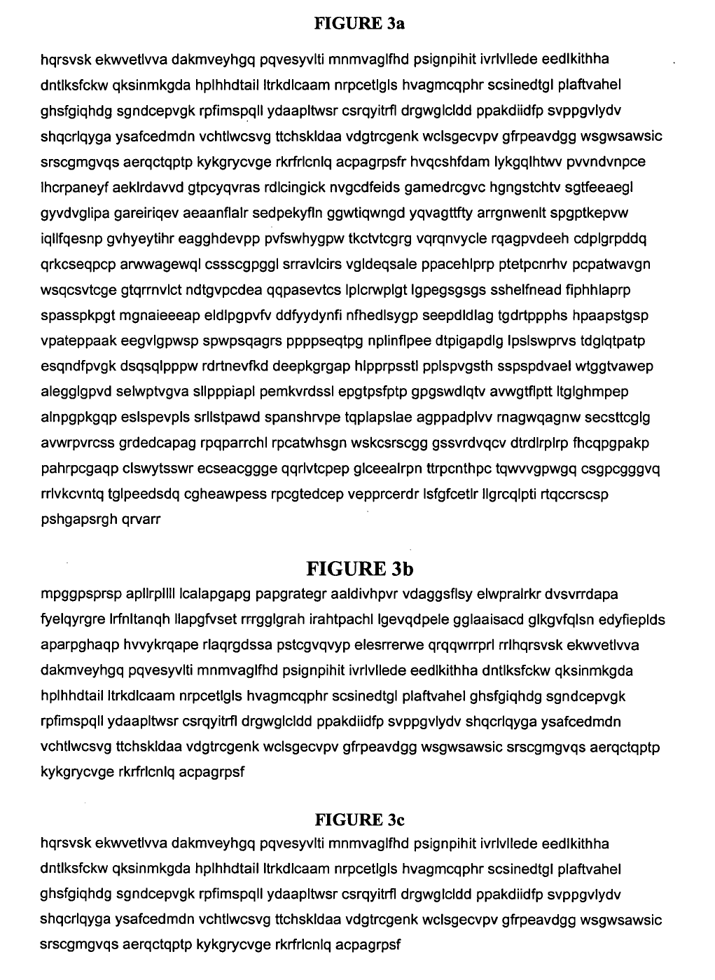

In one aspect of the invention, a truncated ADAMTS with aggrecanase activity is a truncated ADAMTS-7. In one embodiment, the truncation deletes the cysteine-rich, spacer, and five C-terminal TSR domains of ADAMTS-7. Full length ADAMTS-7 is set forth by SEQ ID NO:1 (GenBank Accession No. NP_{—}055087). In a particular embodiment, the truncated ADAMTS-7 molecule lacks the prodomain and comprises, consists essentially of, or consists of amino acids 233-1686, as set forth in SEQ ID NO:2 (FIG. 3a). In another particular embodiment, the truncated ADAMTS 7 lacks the c-terminus after the conserved Phe and comprises, consists essentially of, or consists of amino acids 1-599, as set for in SEQ ID NO:3 (FIG. 3b). In further particular embodiment, the truncated ADAMTS-7 molecule lacks the protein domain and the c-terminus after the conserved Phe and comprises, consists essentially of, or consists of amino acids 233-599, as set forth in SEQ ID NO:4 (FIG. 3c).

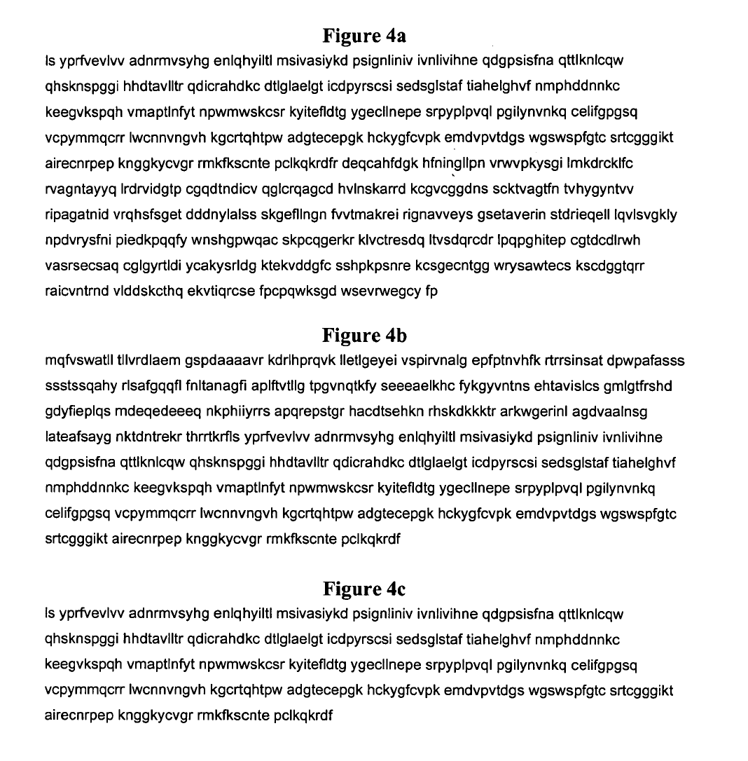

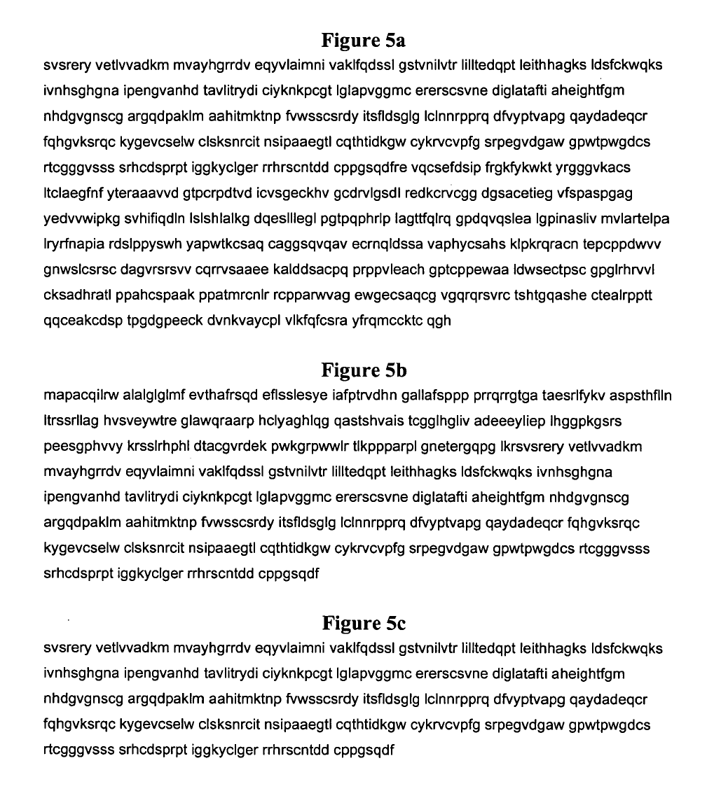

In another aspect of the invention, a truncated ADAMTS with aggrecanase activity is a truncated ADAMTS-9. Full length ADAMTS-9 is set forth by SEQ ID NO:5 (GenBank Accession No. AAF89106). In one embodiment, the truncation deletes the cysteine-rich, spacer, and two C-terminal TSR domains of ADAMTS-9. In a particular embodiment, the truncated ADAMTS-9 lacks the prodomain and comprises, consists essentially of, or consists of amino acids 288-1072, as set forth in SEQ ID NO:6 (FIG. 4a). In another particular embodiment, the truncated ADAMTS-9 lacks the c-terminus after the conserved Phe and comprises, consists essentially of, or consists of amino acids 1-649, as set forth by SEQ ID NO:7 (FIG. 4b). In a further embodiment, the truncated ADAMTS-9 lacks the c-terminus after the conserved Phe and the prodomain and comprises, consists essentially of, or consists of amino acids 288-649, as set forth by SEQ ID NO:8 (FIG. 4c).

In a further aspect of the invention, a truncated ADAMTS with aggrecanase activity is a truncated ADAMTS-10. In one embodiment, the truncation deletes the cysteine-rich, spacer, and five C-terminal TSR domains of ADAMTS-10. Full length ADAMTS-10 is set forth in SEQ ID NO:9 (GenBank Accession No. NP_{—}112219). In a particular embodiment, the truncated ADAMTS-10, lacks the prodomain and comprises, consists essentially of, or consists of amino acids 234-1103, as set forth in SEQ ID NO:10 (FIG. 5a). In another particular embodiment, the truncated ADAMTS-10 lacks the c-terminus after the conserved Phe and comprises, consists essentially of, or consist of amino acids 1-608, as set forth in SEQ ID NO:11 (FIG. 5b) the truncated ADAMTS-10, lacks the prodomain and comprises, consists essentially of, or consists of amino acids 234-1103, as set forth in SEQ ID NO:11 (FIG. 5b). In another embodiment, the truncated ADAMTS-10 lacks the c-terminus after the conserved Phe and the prodomain and comprises, consists essentially of, or consists of amino acids 234-608, as set forth in SEQ ID NO:12 (FIG. 5c).

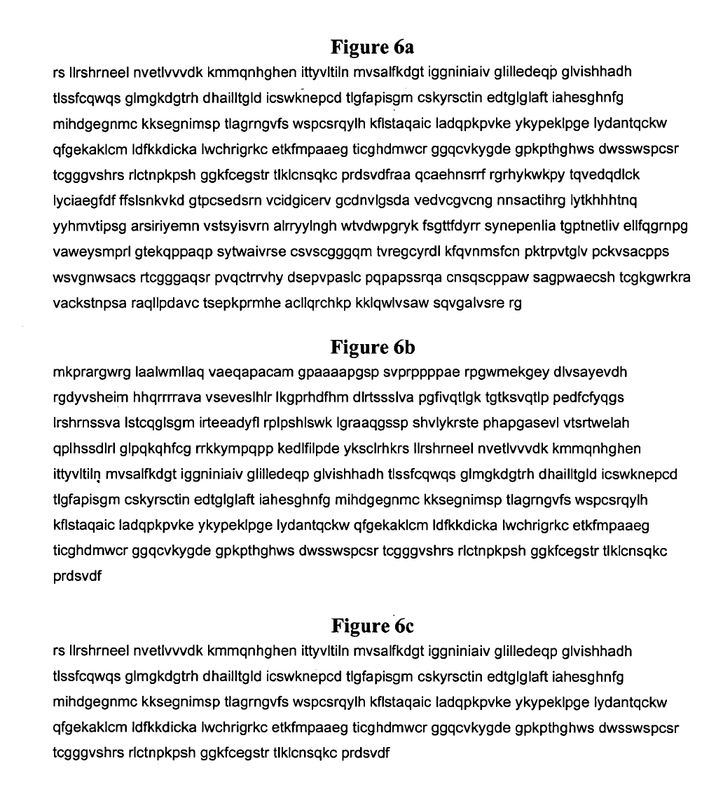

In another aspect of the invention, a truncated ADAMTS with aggrecanase activity is a truncated ADAMTS-16. In one embodiment, the truncation deletes the cysteine-rich, spacer, and two C-terminal TSR domains of ADAMTS-16. Full length ADAMTS-16 is set forth in SEQ ID NO:13 (GenBank Accession No. NP_{—}620687). In a particular embodiment, the truncated ADAMTS-16 lacks the prodomain and comprises, consists essentially of, or consists of amino acids 279-1072, as set forth in SEQ ID NO:14 (FIG. 6a). In another particular embodiment, the truncated ADAMTS-16 lacks the c-terminus after the conserved Phe and comprises, consists essentially of, or consists of amino acids 1-647, as set forth in SEQ ID NO:15 (FIG. 6b). In a further particular embodiment, the truncated ADAMTS-16, lacks the prodomain and the c-terminus after the conserved Phe and comprises, consists essentially of, or consists of amino acids 279-647, as set forth in SEQ ID NO:16 (FIG. 6c).

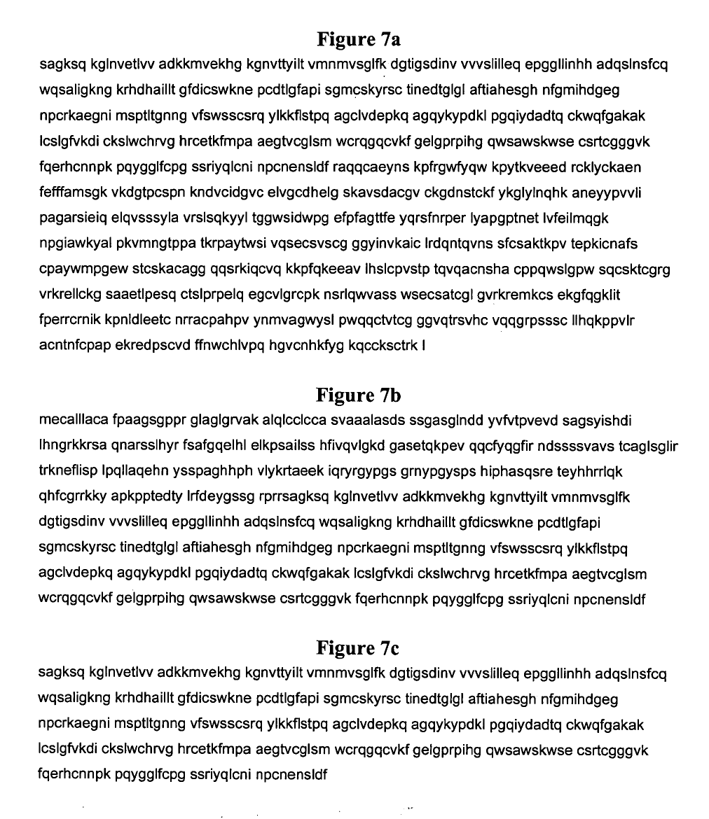

In a further aspect of the invention, a truncated ADAMTS with aggrecanase activity is a truncated ADAMTS-18. In one embodiment, the truncation deletes the cysteine-rich, spacer, and five C-terminal TSR domains of ADAMTS-18. Full length ADAMTS-18 is set forth in SEQ ID NO:17 (GenBank Accession No. NP_{—}955387). In a particular embodiment, the truncated ADAMTS-18 lacks the prodomain and comprises, consists essentially of, or consists of amino acids 285-1221, as set forth in SEQ ID NO:18 (FIG. 7a). In another particular embodiment, the truncated ADAMTS-18 lacks the c-terminus after the conserved Phe and comprises, consists essentially of, or consists of amino acids 1-650, as set forth in SEQ ID NO:19 (FIG. 7b). In a further particular embodiment, the truncated ADAMTS-18 lacks the the c-terminus after the conserved Phe and the prodomain and comprises, consists essentially of, or consists of amino acids 285-650, as set forth in SEQ ID NO:20 (FIG. 7c).

In addition to the proteins described above, the truncated biologically active ADAMTS proteins provided herein also include those with amino acid sequences similar to those set forth in SEQ ID NOs:2-4,6-8, 10-12, 14-16, and 18-20 but into which insertions, deletions, or substitutions have been naturally provided (i.e., allelic variants) or deliberately engineered. For example, numerous conservative substitutions between functionally similar amino acids (e.g., acidic, basic, branched, etc.) are possible without significantly affecting the structure or activity of the truncated proteins described above.

In one embodiment, an aggrecanase of the present invention is obtainable by deleting from a full-length ADAMTS protein at least a substantial portion of the cysteine-rich domain. For instance, the deletion can include, without limitation, at least 30%, 40%, 50%, 60%, 70%, 80%, 90%, 95%, 96%, 97%, 98%, 99% or 100% of the amino acid residues of the cysteine-rich domain. Amino acid residues from other regions or ancillary domains can also be deleted. These other regions or ancillary domains include, for example, the disintegrin-like domain, the central thrombospondin type I repeat, the spacer domain, any C-terminal thrombospondin type I repeat, any regions located between or after the ancillary domains, the signal peptide, and the prodomain.

In another embodiment, an aggrecanase of the present invention is obtainable by deleting from a full-length ADAMTS protein a substantial portion of the amino acid residues that are located C-terminal to a spatially conserved phenylalanine residue after the central thrombospondin type I repeat. As used herein, a conserved residue is shared by at least the majority of the ADAMTS family members. For instance, a conserved residue can be shared by at least 60%, 70%, 80%, 90%, 95% or 100% of all of the ADAMTS family members. A conserved residue can be identified using various methods known in the art. In one example, an optimal sequence alignment is first generated for different ADAMTS family members. Algorithms suitable for this purpose include, but are not limited to, CLUSTALW, MSA, PRALINE, DIALIGN, PRRP, SAGA, and MACAW. See Mount, BIOINFORMATICS (Cold Spring Harbor Laboratory Press, New York, 2001), p. 141. Conserved residues shared by at least the majority of the ADAMTS family members can be identified. Other approaches can also be employed to identify conserved residues.

The deletion utilized can encompass any residue or sequence fragment located C-terminal to the first conserved phenylalanine residue after the central thrombospondin type I repeat. For instance, the deleted amino acid residues can be selected from the cysteine-rich domain, the spacer domain, the C-terminal thrombospondin domain(s), or any region located therebetween or thereafter. The deleted residues can include residues from one or more domains. The deletion of a domain can be either complete or partial.

In one example, the deletion includes at least 30% of the total amino acid residues located C-terminal to the first conserved phenylalanine residue. For instance, the deletion can include at least 40%, 50%, 60%, 70%, 80%, 90% or 100% of all of the amino acid residues located C-terminal to the conserved phenylalanine residue. The deleted residues can include one or more consecutive sequence fragments. Each deleted sequence fragment can include, for example, from 2 to 5 amino acids, from 5 to 10 amino acids, from 10 to 20 amino acids, from 20 to 30 amino acids, from 30 to 50 amino acids, from 50 to 100 amino acids, from 100 to 150 amino acids, from 150 to 200 amino acids, from 200 to 250 amino acids, from 250 to 300 amino acids, from 300 to 350 amino acids, from 350 to 400 amino acids, from 400 to 450 amino acids, from 450 to 500 amino acids, or greater than 500 amino acids. In addition, the deleted residues can include nonconsecutive residues.

In still yet another embodiment, the full-length ADAMTS protein, from which an aggrecanase of the present invention can be derived, is a naturally-occurring full-length ADAMTS protein. The naturally-occurring full-length protein includes ADAMTS isoforms produced by alternative RNA splicing. The full-length ADAMTS protein can be a pro-protein which includes a signal peptide or a prodomain. The full-length ADAMTS protein can also be a mature proteins which lacks the signal peptide and prodomain.

In another embodiment, the full-length ADAMTS protein, from which an aggrecanase of the present invention can be derived, is a variant of a naturally-occurring full-length ADAMTS protein. The amino acid sequence of the variant is substantially identical to that of the naturally-occurring protein. In one example, the amino acid sequence of the variant has at least 80%, 85%, 90%, 95%, 99%, or more global sequence identity or similarity to the naturally-occurring protein. Sequence identity or similarity can be determined using various methods known in the art. For instance, sequence identity or similarity can be determined using standard alignment algorithms, such as Basic Local Alignment Tool (BLAST) described in Altschul, et al., J. MOL. BIOL., 215:403-410 (1990), the algorithm of Needleman, et al., J. MOL. BIOL., 48:444-453 (1970), the algorithm of Meyers, et al., COMPUT. APPL. BIOSCI., 4:11-17(1988), and dot matrix analysis. Suitable sequence alignment programs include, but are not limited to, BLAST programs provided by the National Center for Biotechnology Information (Bethesda, Md.) and MegAlign provided by DNASTAR, Inc. (Madison, Wis.). In one instance, the sequence identity or similarity is determined by using the Genetics Computer Group (GCG) programs GAP (Needleman-Wunsch algorithm). Default values assigned by the programs are employed (e.g., the penalty for opening a gap in one of the sequences is 11 and for extending the gap is 8). Similar amino acids can be defined using the BLOSUM62 substitution matrix.

In one example, the naturally-occurring ADAMTS protein and its variant can be substantially identical in one or more regions, but divergent in others. In another example, the variant retains the overall domain structure of the naturally-occurring protein. In yet another example, the variant is prepared by making at least 1, 2, 3, 4, 5, 10, 15, 20, 25, 30, 35, 40, 45, 50, or more amino acid substitutes, deletions, or insertions in the naturally-occurring sequence. The substitutions can be conservative, non-conservative, or both. The aggrecanases of the present invention can be pro-proteins that include a signal peptide or a prodomain. The aggrecanases of the present invention can also be mature proteins that lack any signal peptide or prodomain.

The aggrecanases of the present invention can also include deletions located N-terminal to the first consecutive phenylalanine residue after the central thrombospondin type I repeat. For instance, certain residues in the metalloprotease catalytic domain, the disintegrin-like domain, or the central thrombospondin type I repeat can be deleted without abolishing or significantly changing the aggrecanase activity of the original protein. The deleted residues may or may not be involved in aggrecan binding or proteolytic activities.

The present invention also contemplates variants of the above-described aggrecanases. These variants have aggrecanase activities that can be readily determined using the assays described below. Variants in a protein sequence can be naturally occurring, such as by allelic variations or polymorphisms, or deliberately engineered. Numerous conservative amino acid substitutions can be introduced into a protein sequence without significantly changing the structure or biological activity of the protein. Conservative amino acid substitutions can be made on the basis of similarity in polarity, charge, solubility, hydrophobicity, hydrophilicity, or the amphipathic nature of the residues. For instance, conservative amino acid substitutions can be made among amino acids with basic side chains, such as lysine (Lys or K), arginine (Arg or R) and histidine (His or H); amino acids with acidic side chains, such as aspartic acid (Asp or D) and glutamic acid (Glu or E); amino acids with uncharged polar side chains, such as asparagine (Asn or N), glutamine (Gln or Q), serine (Ser or S), threonine (Thr or T), and tyrosine (Tyr or Y); and amino acids with nonpolar side chains, such as alanine (Ala or A), glycine (Gly or G), valine (Val or V), leucine (Leu or L), isoleucine (Ile or I), proline (Pro or P), phenylalanine (Phe or F), methionine (Met or M), tryptophan (Trp or W) and cysteine (Cys or C). Other exemplary amino acid substitutions are illustrated in Table 1.

TABLE 1
Exemplary Amino Acid Substitutions

| | | More |
|---|---|---|
| Original | | Conservative |
| Residues | Exemplary Substitutions | Substitutions |
| Ala (A) | Val, Leu, Ile | Val |
| Arg (R) | Lys, Gln, Asn | Lys |
| Asn (N) | Gln | Gln |
| Asp (D) | Glu | Glu |
| Cys (C) | Ser, Ala | Ser |
| Gln (Q) | Asn | Asn |
| Gly (G) | Pro, Ala | Ala |
| His (H) | Asn, Gln, Lys, Arg | Arg |
| Ile (I) | Leu, Val, Met, Ala, Phe, Norleucine | Leu |
| Leu (L) | Norleucine, Ile, Val, Met, Ala, Phe | Ile |
| Lys (K) | Arg, 1,4 Diamino-butyric Acid, Gln, Asn | Arg |
| Met (M) | Leu, Phe, Ile | Leu |
| Phe (F) | Leu, Val, Ile, Ala, Tyr | Leu |
| Pro (P) | Ala | Gly |
| Ser (S) | Thr, Ala, Cys | Thr |
| Thr (T) | Ser | Ser |
| Trp (W) | Tyr, Phe | Tyr |
| Tyr (Y) | Trp, Phe, Thr, Ser | Phe |
| Val (V) | Ile, Met, Leu, Phe, Ala, Norleucine | Leu |

Normaturally occurring amino acid residues can be used for conservative substitutions. These amino acid residues are typically incorporated by chemical peptide synthesis rather than by synthesis in biological systems.

In addition, aggrecanase variants can include amino acid substitutions to increase the stability of the molecules. For example, an E-to-Q mutation at position 411 in the catalytic domain of an aggrecanase molecule may increase stability and half-life of the aggrecanase. Amino acid mutations in other regions of an aggrecanase can be also employed to increase the stability of the molecule.

Other desirable amino acid substitutions (whether conservative or nonconservative) can be also introduced into the aggrecanase molecules. For instance, amino acid residues important to the biological activity of an aggrecanase molecule can be identified. Substitutions capable of increasing or decreasing the aggrecanase activity can then be selected.

Furthermore, aggrecanase variants can include modifications of glycosylation sites. These modifications can involve O-linked or N-linked glycosylation sites. For instance, the amino acid residues at asparagine-linked glycosylation recognition sites can be substituted or deleted, resulting in partial glycosylation or complete abolishment of glycosylation. The asparagine-linked glycosylation recognition sites typically comprise tripeptide sequences that are recognized by appropriate cellular glycosylation enzymes. These tripeptide sequences can be either asparagine-X-threonine or asparagine-X-serine, where X is usually any amino acid. A variety of amino add substitutions or deletions at one or both of the first or third amino acid positions of a glycosylation recognition site (or amino acid deletion at the second position) can result in non-glycosylation at the modified tripeptide sequence. Additionally, bacterial expression of an aggrecanase-related protein also results in production of a non-glycosylated protein, even if the glycosylation sites are left unmodified.

Aggrecanase variants can also be prepared by incorporating other modifications into the original polypeptide. These modifications can be introduced by naturally-occurring processes, such as posttranslational modifications, or by artificial or synthetic processes. Suitable modifications can occur anywhere in the polypeptide, including the backbone, the amino acid side chains, and the amino or carboxyl termini. The same type of modification can be present in the same or varying degrees at several sites in a variant. A variant can also contain many different types of modifications. Exemplary modifications suitable for this invention include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, ubiquitination, or any combination thereof. A polypeptide variant can be branched, for example, as a result of ubiquitination, or it may be cyclic, with or without branching.

In yet another embodiment, the aggrecanases of the present invention are obtainable from a full-length ADAMTS protein by modifying the amino acid residues that are deletable according to the present invention. Exemplary modifications include, but are not limited to, substitutions and insertions. In one example, the modifications substantially transform an ancillary domain or a fragment thereof such that the ancillary domain or fragment is considered deleted from the full-length ADAMTS protein. In another example, the transformed domain or fragment has less than 50%, 40%, 30%, 20%, 10% or 5% sequence identity or similarity to the original domain or fragment. In a further example, the modifications include at least an insertion of a sequence after the first conserved phenylalanine residue after the central thrombospondin type I repeat. The domains that are located N-terminal to the inserted sequence retain aggrecanase activity and therefore constitute a separable aggrecanase unit.

In many embodiments, the aggrecanases of the present invention are in isolated or purified forms. In one example, an aggrecanase preparation of the present invention is substantially free from other proteins. For instance, the aggrecanase preparation can include less than 80%, 70%, 60%, 50%, 40%, 30%, 20%, 10%, 5% or 1% by weight of other proteins. In another example, the aggrecanase preparation contains an insignificant amount of contaminants that would otherwise interfere with the intended use of the aggrecanase.

The aggrecanases of the present invention have proteolytic activity and preferably cleave the Glu³⁷³-Ala³⁷⁴ bond in the IGD of aggrecan. In one example, an aggrecanase of the present invention retains a substantial portion of the aggrecanase activity of the full-length ADAMTS protein from which the aggrecanase can be derived. For instance, the aggrecanase can retain at least 10%, 20%, 30%, 40%, 50%, 60%, 70%, 80%, 90% or 100% of the aggrecanase activity of the full-length ADAMTS protein. In another example, an aggrecanase of the present invention possesses a higher aggrecanase activity than that of the full-length ADAMTS protein. In yet another embodiment, the full-length ADAMTS protein has no detectable aggrecanase activity, and deletion of numerous amino acid residues from the full-length protein confers aggrecanase activity to the modified protein.

The present invention also provides polynucleotides encoding novel truncated forms of biologically active ADAMTS proteins, particularly those with aggrecanase activity.

In one aspect of the invention, a polynucleotide encodes truncated ADAMTS-7. Preferably, the polynucleotide encodes a truncated ADAMTS-7 molecule in which the cysteine-rich, spacer, and five C-terminal TSR domains are deleted. In a particular embodiment, the polynucleotide encoding truncated ADAMTS-7 lacks the region which encodes the prodomain and comprises, consists essentially of, or consists of nucleic acids 699-5058, as set forth in SEQ ID NO:21. In another embodiment, the polynucleotide encoding truncated ADAMTS-7 lacks the region which encodes the c-terminus after the conserved Phe and comprises, consists essentially of, or consists of nucleic acids 1-1797, as set forth in SEQ ID NO:22. In a further embodiment, the polynucleotide encoding truncated ADAMTS-7 lacks both the prodomain and the c-terminus after the conserved Phe and comprises, consists essentially of, or consists of nucleic acids 699-1797, as set forth in SEQ ID NO:23.

In another aspect of the invention, a polynucleotide encodes truncated ADAMTS-9. Preferably, the polynucleotide encodes a truncated ADAMTS-9 molecule in which the cysteine-rich, spacer, and two C-terminal TSR domains are deleted. In a particular embodiment, the polynucleotide encoding truncated ADAMTS-9 lacks the prodomain and comprises, consists essentially of, or consists of nucleotides 864-3216, as set forth in SEQ ID NO:24. In another embodiment, the polynucleotide encoding truncated ADAMTS-9 lacks the region which encodes the c-terminus after the conserved Phe and comprises, consists essentially of, or consists of nucleic acids 1-1947, as set forth in SEQ ID NO:25. In a further embodiment, the polynucleotide encoding truncated ADAMTS-9 lacks both the prodomain and the c-terminus after the conserved Phe and comprises, consists essentially of, or consists of nucleic acids 864-1947, as set forth in SEQ ID NO:26.

In further aspect of the invention, a polynucleotide encodes truncated ADAMTS-10. Preferably, the polynucleotide encodes a truncated ADAMTS-10 molecule in which the cysteine-rich, spacer, and five C-terminal TSR domains are deleted. In a particular embodiment, the polynucleotide encoding truncated ADAMTS-10 lacks the prodomain and comprises, consists essentially of, or consists of nucleic acids 702-3309, as set forth in SEQ ID NO:27. In another embodiment, the polynucleotide encoding truncated ADAMTS-10 lacks the region encoding the c-terminus after the conserved Phe and comprises, consists essentially of, or consists of nucleotides 1-1824, as set for in SEQ ID NO:28. In a further embodiment, the polynucleotide encoding truncated ADAMTS-10 lacks the region encoding both the prodomain and the c-terminus after the conserved Phe and comprises, consists essentially of, or consists of polynucleotides 702-1824, as set forth in SEQ ID NO:29.

In another aspect of the invention, a polynucleotide encodes truncated ADAMTS-16. Preferably, the polynucleotide encodes a truncated ADAMTS-16 molecule in which the cysteine-rich, spacer, and five C-terminal TSR domains are deleted. In a particular embodiment, the polynucleotide encoding truncated ADAMTS-16 lacks the prodomain and comprises, consists essentially of, or consists of nucleic acids 837-3216, as set forth in SEQ ID NO:30. In another embodiment, the polynucleotide encoding truncated ADAMTS-16 lacks the region encoding the c-terminus after the conserved Phe and comprises, consists essentially of, or consists of nucleotides 1-1941, as set for in SEQ ID NO:31. In a further embodiment, the polynucleotide encoding truncated ADAMTS-16 lacks the region encoding both the prodomain and the c-terminus after the conserved Phe and comprises, consists essentially of, or consists of polynucleotides 837-1941, as set forth in SEQ ID NO:32.

In another aspect of the invention, a polynucleotide encodes truncated ADAMTS-18. Preferably, the polynucleotide encodes a truncated ADAMTS-18 molecule in which the cysteine-rich, spacer, and five C-terminal TSR domains are deleted. In a particular embodiment, the polynucleotide encoding truncated ADAMTS-18 lacks the prodomain and comprises, consists essentially of, or consists of nucleic acids 855-3663, as set forth in SEQ ID NO:33. In another embodiment, the polynucleotide encoding truncated ADAMTS-18 lacks the region encoding the c-terminus after the conserved Phe and comprises, consists essentially of, or consists of nucleotides 1-1950, as set for in SEQ ID NO:34. In a further embodiment, the polynucleotide encoding truncated ADAMTS-18 lacks the region encoding both the prodomain and the c-terminus after the conserved Phe and comprises, consists essentially of, or consists of polynucleotides 855-1950, as set forth in SEQ ID NO:35.

The polynucleotides of the present invention also include those with nucleotide sequences that differ in codon sequence from those set forth above, but which encode a protein that consists of the amino acid sequence set forth in SEQ ID NOs:2-4,6-8, 10-12, 14-16, and 18-20 (e.g., due to the well-known degeneracy of the genetic code).

In addition to the polynucleotides encoding truncated biologically active ADAMTS proteins described above, the polynucleotides of the present invention also include those that hybridize under stringent (preferably highly stringent) conditions to the nucleotide sequences set forth in SEQ ID NOs: 21-35. Such polynucleotides include those with nucleotide sequences similar to the polynucleotides set forth in SEQ ID NOs: 21-35, but into which insertions, deletions, or substitutions have been naturally provided (i.e., allelic variants) or deliberately engineered. Preferably, allelic variants of the present invention have at least 90% sequence identity (more preferably, at least 95% identity; most preferably, at least 99% identity) with the nucleotide sequences set forth in SEQ ID NOs: 21-35.

The polynucleotides of the present invention that hybridize under stringent conditions to the nucleotide sequences set forth in SEQ ID NOs: 21-35 also include those with sequences homologous to the disclosed polynucleotides. These homologs are polynucleotides (and translated polypeptides) isolated from a different species than those of the disclosed polynucleotides (and translated polypeptides), or within the same species, but with significant sequence similarity to the disclosed polynucleotides (and translated polypeptides). Preferably, the polynucleotide homologs have at least 60% sequence identity (more preferably, at least 75% identity; most preferably, at least 90% identity) with the disclosed polynucleotides and are isolated from mammalian species (more preferably primate, most preferably human).

Hybridization conditions of high stringency are well known in the art. Examples of various stringency conditions are shown in Table 2 below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE 2

| Stringency | Polynucleotide | Hybrid | Hybridization Temperature | Wash Temperature |
|---|---|---|---|---|
| Condition | Hybrid | Length (bp)¹ | and Buffer² | and Buffer² |
| A | DNA:DNA | >50 | 65° C.; 1 × SSC -or- | 65° C.; 0.3 × SSC |
| | | | 42° C.; 1 × SSC, 50% | |
| | | | formamide | |
| B | DNA:DNA | <50 | T_{B}*; 1 × SSC | T_{B}*; 1 × SSC |
| C | DNA:RNA | >50 | 67° C.; 1 × SSC -or- | 67° C.; 0.3 × SSC |
| | | | 45° C.; 1 × SSC, 50% | |
| | | | formamide | |
| D | DNA:RNA | <50 | T_{D}*; 1 × SSC | T_{D}*; 1 × SSC |
| E | RNA:RNA | >50 | 70 C.; 1 × SSC | 70° C.; 0.3 × SSC |
| | | | -or- | |
| | | | 50° C.; 1 × SSC, 50% | |
| | | | formamide | |
| F | RNA:RNA | <50 | T_{F}*; 1 × SSC | T_{F}*; 1 × SSC |
| G | DNA:DNA | >50 | 65° C.; 4 × SSC | 65° C.; 1 × SSC |
| | | | -or- | |
| | | | 42° C.; 4 × SSC, 50% | |
| | | | formamide | |
| H | DNA:DNA | <50 | T_{H}*; 4 × SSC | T_{H}*; 4 × SSC |
| I | DNA:RNA | >50 | 67° C.; 4 × SSC | 67° C.; 1 × SSC |
| | | | -or- | |
| | | | 45° C.; 4 × SSC, 50% | |
| | | | formamide | |
| J | DNA:RNA | <50 | T_{J}*; 4 × SSC | T_{J}*; 4 × SSC |
| K | RNA:RNA | >50 | 70° C.; 4 × SSC | 67° C.; 1 × SSC |
| | | | -or- | |
| | | | 50° C.; 4 × SSC, 50% | |
| | | | formamide | |
| L | RNA:RNA | <50 | T_{L}*; 2 × SSC | T_{L}*; 2 × SSC |
| M | DNA:DNA | >50 | 50° C.; 4 × SSC | 50° C.; 2 × SSC |
| | | | -or- | |
| | | | 40° C.; 6 × SSC, 50% | |
| | | | formamide | |
| N | DNA:DNA | <50 | T_{N}*; 6 × SSC | T_{N}*; 6 × SSC |
| O | DNA:RNA | >50 | 55° C.; 4 × SSC | 55° C.; 2 × SSC |
| | | | -or- | |
| | | | 42° C.; 6 × SSC, 50% | |
| | | | formamide | |
| P | DNA:RNA | <50 | T_{P}*; 6 × SSC | T_{P}*; 6 × SSC |
| Q | RNA:RNA | >50 | 60° C.; 4 × SSC -or- | 60° C.; 2 × SSC |
| | | | 45° C.; 6 × SSC, 50% | |
| | | | formamide | |
| R | RNA:RNA | <50 | T_{R}*; 4 × SSC | T_{R}*; 4 × SSC |

¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity.
²SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes after hybridization is complete. T_{B}*−T_{R}*: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀Na⁺)+0.41(% G+C)−(600/N), where N is the number of bases in the hybrid, and Na⁺ is the concentration of sodium ions in the hybridization buffer (Na⁺ for 1×SSC=0.165M).

Additional examples of stringency conditions for polynucleotide hybridization are provided in Sambrook et al., Molecular Cloning: A Laboratory Manual, Chs. 9 & 11, Cold Spring Harbor Laboratory Press, Cold Spring Harbor, N.Y. (1989), and Ausubel et al., eds., Current Protocols in Molecular Biology, Sects. 2.10 & 6.3-6.4, John Wiley & Sons, Inc. (1995), herein incorporated by reference.

Polynucleotides encoding the aggrecanases of the present invention can be prepared using a variety of methods. For instance, the coding sequence for an aggrecanase of the present invention can be derived from the cDNA sequence of a full-length ADAMTS by one or more deletions. For published full-length ADAMTS cDNA sequences, see, for example, Tortorella, et al., SCIENCE, 284:1664-1666 (1999); Hurskainen, et al., supra; Clark, et al., GENOMICS, 67:343-350 (2000); and Cal, et al., GENE, 283:49-62 (2002). Deletions from a full-length ADAMTS cDNA sequence can be prepared using numerous methods.

In one embodiment, deletion of a sequence located between two selected fragments is prepared using PCR-mediated reactions. The selected fragments can be first PCR amplified and then in-frame ligated, thereby deleting the sequence located therebetween. The ligation product can be subcloned into a vector for expression in host cells. In another embodiment, a truncated ADAMTS can be produced by PCR amplifying only the desired portion of the ADAMTS coding sequence. In yet another embodiment, the deletion is based on two naturally-occurring or genetically engineered restriction endonuclease recognition sites in an ADAMTS coding sequence. Desired restriction sites can be introduced into the ADAMTS coding sequence by any traditional means, such as site-directed mutagenesis. Cleavage at the two restriction sites and subsequent in-frame ligation will delete the sequence located between the two restriction sites. Other deletion methods, such as oligonucleotide-directed “loop-out” mutagenesis, PCR overlap extension, time-controlled digestion with exonuclease III, the megaprimer procedure, inverse PCR, or automated DNA synthesis, can also be employed.

Deletions can be introduced into any region in an ADAMTS coding sequence. The modified ADAMTS protein can differ from a full-length ADAMTS protein by two or more deletions. Deletions can occur in the same domain or different domains of an ADAMTS protein.

In one embodiment, a deletion library is generated. The deletion library can include coding sequences for N-terminal, C-terminal, or internal deleted ADAMTS proteins. An exemplary method for this purpose is described in Pues, et al., NUCLEIC ACIDS RES., 25:1303-1305 (1997). Commercial kits, such as the EZ::TN Plasmid-Based Deletion Machine and the pWEB::TNC™ Deletion Cosmid Transposition Kit (Epicentre, Madison, Wis.), can also be used to generate ADAMTS deletion libraries. Deletions can be verified by DNA or protein sequencing. Deletions that produce biologically active aggrecanases can be selected.

In another embodiment, an ADAMTS fragment is deleted by randomly introducing mutations into the coding sequence of the fragment. Suitable methods for this purpose include, but are not limited to, saturation mutagenesis. Where a stop codon is introduced, the deletion includes all the residues located after the stop codon.

As described above, deletion includes the situations where the deleted amino acid residues or fragments are replaced by other residues or fragments. Such a replacement can be readily achieved at the coding sequence level using various methods known in the art. Other suitable methods can also be employed. Thus, the deletion of a fragment can be created when randomly introduced mutations substantially transform the encoded polypeptide fragment.

Preparation of deletions is not limited to the use of full-length ADAMTS cDNA sequences. Deletions can also be prepared using expression sequence tags or other partial or incomplete cDNA or mRNA sequences. In addition, genomic sequences can be used to produce modified ADAMTSs of the present invention. Moreover, deletions can be carried out by modifying the splice acceptor or donor sites or other functional intron sequences in ADAMTS coding sequences.

Sequences including the degeneracy of the genetic code or other variations can also be employed. There are many polynucleotide variants that encode the same polypeptide as a result of the degeneracy of the genetic code. Some of these polynucleotide variants bear minimal sequence identity to the original polynucleotide. Nonetheless, the present invention contemplates the use of polynucleotides that vary due to differences in codon usage.

The nucleic acid sequences that encode other polypeptides can be in-frame fused to the 5′ or 3′ end of the aggrecanase coding sequence. These additional polypeptides can be, for example, peptide tags, enzymes, ligand/receptor binding proteins, antibodies, or any combination thereof.

The polynucleotides of the present invention can be modified to increase stability in vivo. Possible modifications include, but are not limited to, the addition of flanking sequences at the 5′ or 3′ end; the use of phosphorothioate or 2-o-methyl instead of phosphodiesterase linkages in the backbone; and the inclusion of nontraditional bases such as inosine, queosine and wybutosine, as well as acetyl-, methyl-, thio-, or other modified forms of adenine, cytidine, guanine, thymine and uridine.

The polynucleotides of the present invention can be DNA, RNA, or other expressible nucleic acid molecules. The polynucleotides can be single-stranded or double-stranded.

In one embodiment, the polynucleotides of the present invention are expression vectors comprising 5′ or 3′ untranslated regulatory sequences operatively linked to the sequence encoding an aggrecanase of the present invention. In another embodiment, the aggrecanases of the present invention are expressed from expression vectors without undergoing any C-terminal proteolytic cleavage.

Expression vectors commonly include one or more selectable markers and one or more origins of replication, although those skilled in the art will recognize that within certain systems selectable markers can be provided on separate vectors, and replication of the exogenous DNA can be provided by integration into the host cell genome. The design of expression vectors depends on such factors as the choice of the host cells or the desired expression levels. Selection of promoters, enhancers, selectable markers, and other elements is a matter of routine design within the level of ordinary skill in the art. Many such elements are described in the literature and are available through commercial suppliers.

Expression vectors can be derived from a variety of sources, such as plasmids, viruses, or any combination thereof. Suitable viral vectors include, but are not limited to, retroviral, lentiviral, adenoviral, adeno-associated viral (AAV), herpes viral, alphavirus, astrovirus, coronavirus, orthomyxovirus, papovavirus, paramyxovirus, parvovirus, picornavirus, poxvirus, or togavirus vectors.

In one embodiment, the expression vector is an E. coli vector which has a constitutive or inducible promoter. Sequences encoding additional peptides can be fused to the aggrecanase coding sequence in order to serve desirable purposes, such as increasing the expression or solubility of the recombinant protein or aiding its purification. In one example, the fused peptide(s) is cleavable from the recombinant protein. Expression vectors suitable for this purpose include, but are not limited to, pGEX (Pharmacia Piscataway, N.J.), pMAL (New England Biolabs, Beverly, Mass.), and pRITS (Pharmacia, Piscataway, N.J.).

Various methods can be used to maximize the expression of the recombinant protein in E. coli. One strategy is to use a host bacterium that has an impaired capacity to proteolytically cleave the recombinant protein. Another strategy is to alter the coding sequence such that the individual codon for each amino acid is preferentially utilized by E. coli.

In another embodiment, the expression vector is a yeast expression vector. Exemplary yeast expression vectors include, but are not limited to, pYepSec1, pMFa, pJRY88, pYES2 (Invitrogen Corporation, San Diego, Calif.), and picZ (Invitrogen Corp, San Diego, Calif.).

In yet another embodiment, the expression vector is an insect cell expression vector. Commonly used insect cell expression vectors include baculovirus expression vectors, such as the pAc and pVL series.

In still another embodiment, the expression vector is a mammalian expression vector. Suitable mammalian expression vectors include, but are not limited to, pCDM8, pMT2PC, pJL3, pJL4, pMT2 CXM, and pEMC2β1. When used in mammalian cells, the expression control sequences are often provided by viral regulatory elements. For example, promoters derived from polyoma, adenovirus 2, cytomegalovirus, or Simian virus 40 are commonly employed in mammalian expression vectors.

The mammalian expression vector of the present invention can also include tissue-specific regulatory elements. Suitable tissue-specific promoters include, but are not limited to, liver-specific promoters, lymphoid-specific promoters, T cell-specific promoters, neuron-specific promoters, pancreas-specific promoters, and mammary gland-specific promoters. In addition, the present invention contemplates the use of developmentally-regulated promoters, such as the α-fetoprotein promoter. The expression of ADAMTSs has been detected in numerous tissues and at various developmental stages. For instance, Northern blot analysis showed that ADAMTS-9 is highly expressed in adult heart, placenta, and skeletal muscle, but has low to undetectable levels in spleen, thymus, prostate, testis, small intestine, and peripheral blood leukocytes. See Somerville, et al., J. BIOL. CHEM., 278:9503-9513 (2003). RT-PCR analysis also detected ADAMTS-9 expression in ovary, pancreas, lung, and kidney. During development, expression of ADAMTS-9 is high in 7- and 17-day-old mouse embryos and lower in 11- and 15-day-old mouse embryos. Likewise, ADAMTS-7 has been detected in a variety of tissues, such as brain, heart, lung, liver, pancreas, kidney, skeletal muscle, and placenta. See Hurskainen, et al., supra. The use of tissue-specific or developmentally-regulated promoters allows more specific functional analyses of ADAMTS proteins.

In still yet another embodiment, the expression vector includes the ADAMTS coding sequence in an antisense orientation. Regulatory sequences that are operatively linked to the antisense-oriented coding sequence can be chosen to direct continuous expression of the antisense RNA molecule in a variety of cell types. Suitable regulatory sequences include viral promoters or enhancers. Regulatory sequences can also be selected to direct constitutive or tissue specific expression of the antisense RNA.

Moreover, the present invention contemplates the use of regulatable expression systems to express aggrecanases in numerous types of cells. Systems suitable for this purpose include, but are not limited to, the Tet-on/off system, the Ecdysone system, the Progesterone system, and the Rapamycin system. The Tet-on/off system is based on two regulatory elements derived from the tetracycline-resistance operon of the E. coli Tn10 transposon. The system includes two components: a regulator plasmid and a reporter plasmid. The regulator plasmid encodes a hybrid protein containing a mutated Tet repressor (rtetR) fused to the VP16 activation domain of herpes simplex virus. The reporter plasmid contains a tet-responsive element (TRE) which controls the expression of a reporter gene. The rtetR-VP16 fusion protein binds to the TRE, thereby activating the transcription of the reporter gene in the presence of tetracycline. The Tet-on/off system can be incorporated into a variety of viral vectors, such as retroviral, adenoviral, or AAV vectors.

The Ecdysone system is based on the molting induction system in Drosophila. The system uses muristerone A, an analog of the Drosophila steroid hormone ecdysone, to activate gene expression via a heterodimeric nuclear receptor. In certain embodiments, the induced expression level can be at least 200-fold over the basal level with no significant effect on the physiology of the transfected cells.

The Progesterone system is based on the action of the progesterone receptor. The progesterone receptor is a member of the nuclear/steroid receptor superfamily. Upon binding to its hormone ligand (such as progesterone), the receptor binds to the progesterone response element, thereby activating gene transcription. The action of the progesterone receptor can be blocked by binding to mifepristone (RU486), a progesterone antagonist. A chimeric transcription factor can be made by fusing the RU486-binding domain of the progesterone receptor to the yeast GAL4 DNA-binding domain and the HSV VP16 transcriptional activation domain. The chimeric factor is inactive in the absence of RU486. The addition of RU486, however, induces a conformational change, which in turn activates the chimeric factor and allows transcription from a promoter that contains the GAL4-binding site.

The Rapamycin system, also known as the CID system (“chemical inducers of dimerization”), employs the dimerization activity caused by rapamycin. Rapamycin induces heterodimerization of two cellular proteins FKBP12 and FRAP. The Rapamycin system employs two chimeric proteins. The first chimeric protein includes FKBP12 which is fused to a DNA-binding domain that binds to a DNA response element. The second chimeric protein includes FRAP which is fused to a transcriptional activation domain. The addition of rapamycin causes dimerization of the two chimeric proteins, thereby activating gene transcription controlled by the DNA response element.

The present invention also provides methods for producing truncated biologically active ADAMTS proteins, preferably those with aggrecanase activity. For example, a suitable host cell line, transformed or transfected with a polynucleotide of the present invention (e.g., SEQ ID NOs:21-35) under the control of an expression control sequence, can be cultured under conditions such that the truncated ADAMTS protein (e.g., SEQ ID NOs:2-4. 6-8, 10-12, 14-16, and 18-20) is produced. The protein is recovered from the cells or the culture medium and purified, such that the protein is substantially free from other proteins. General methods for expressing and purifying recombinant proteins are well known in the art.

A number of cell lines may act as suitable host cells for recombinant expression of the polypeptides of the truncated ADAMTS proteins. Mammalian host cell lines include, e.g., COS cells, CHO cells, 293T cells, A431 cells, 3T3 cells, CV-1 cells, HeLa cells, L cells, BHK21 cells, HL-60 cells, U937 cells, HaK cells, Jurkat cells, as well as cell strains derived from in vitro culture of primary tissue and primary explants.

The truncated ADAMTS proteins may also be recombinantly produced in insect cells, such as Sf9 cells and Drosophila S2 cells. Materials and methods for Sf9 and S2 expression are commercially available in kit form (e.g., the MaxBac® kit and DESK kits, respectively, Invitrogen, Carlsbad, Calif.).

Alternatively, it may be possible to recombinantly produce the truncated ADAMTS proteins in lower eukaryotes such as yeast or in prokaryotes. Potentially suitable yeast strains include Sshizosaccharomyces cerevisiae, Schizosaccharomyces pombe, Kluyveromyces strains, and Candida strains. Potentially suitable bacterial strains include Escherichia coli, Bacillus subtilis, and Salmonella typhimurium. If the truncated ADAMTS proteins are made in yeast or bacteria, it may be necessary to modify them by, for example, phosphorylation or glycosylation of appropriate sites, in order to obtain functionality. Such covalent attachments may be accomplished using well-known chemical or enzymatic methods.

Additional polypeptides can be fused to the N- or C-terminus of an aggrecanase of the present invention. Various methods are available for making fusion proteins. The fused polypeptide(s) can serve to facilitate protein purification, detection, immobilization, folding, targeting, or other desirable purposes. The fused polypeptide(s) can also serve to increase the expression, solubility, or stability of the recombinant protein. In one embodiment, the fused polypeptide(s) do not significantly affect the proteolytic activity of the aggrecanase.

Exemplary polypeptides suitable for making fusion proteins include, but are not limited to, peptide tags, enzymes, antibodies, receptors, ligand/receptor binding proteins, or any combination thereof. As used herein, an antibody can be, for example, a polyclonal, monoclonal, mono-specific, poly-specific, non-specific, humanized, human, single-chain, chimeric, synthetic, recombinant, hybrid, mutated, grafted, or in vitro generated antibody. An antibody can also be Fab, F(ab′)₂, Fv, scFv, Fd, dAb, or any other antibody fragment that retains the antigen-binding function.

Peptide tags suitable for the present invention include, but are not limited to, the poly-histidine or poly-histidine-glycine tag, the FLAG epitope tag, the KT3 epitope peptide, the flu HA tag polypeptide, the c-myc tag, the Herpes simplex glycoprotein D, beta-galactosidase, maltose binding protein, streptavidin tag, tubulin epitope peptide, the T7 gene 10 protein peptide tag, and glutathione S-transferase. Antibodies against these peptide tags are readily obtainable. Representative antibodies include antibody 12CA5 against the flu HA tag polypeptide, and the 8F9, 3C7, 6E10, G4, B7 and 9E 10 antibodies against the c-myc tag.

In one embodiment, the fused polypeptide(s) has insubstantial sequence identity or similarity to naturally-occurring ADAMTS sequences. For instance, the fused polypeptide(s) can have less than 80%, 70%, 60%, 50%, 40%, 30%, 20%, 10% or 5% sequence identity or similarity to naturally-occurring full-length ADAMTS proteins. Sequence identity or similarity can be determined by using, for example, the GCG BESTFIT (Smith-Waterman algorithm).

In one embodiment, a Strep-tag® (IBA) is covalently attached to the C-terminus of an aggrecanase of the present invention. The Strep-tag has the amino acid sequence “WSHPQFEK” (amino acid residues 4-11 of SEQ ID NO:36), encoded, for example, by nucleotidesTGGAGCCACCCGCAGTTCGAAAAATAA (SEQ ID NO:37). A peptide linker (e.g., “GSA”) can be added between the tag and the aggrecanase to enhance the accessibility of the tag to give GSAWSHPQFEK (SEQ ID NO:38), encoded by nucleotides GGAAGCGCTTGGAGCCACCCGCAGTTCGAAAAATAA (SEQ ID NO:39.

SEQ ID NO:40-44 show the amino acid sequences of exemplary fusion proteins which include modified ADAMTS-7, -9, -10, -16, and -18, respectively, covalently linked to a Strep-tag at the C-terminus.

A proteolytically cleavable site can be introduced at the junction between the fused polypeptide(s) and the aggrecanase. The cleavable site enables separation of the aggrecanase from the fused polypeptide(s) after purification of the recombinant protein. Suitable cleavage enzymes for this purpose include, but are not limited to, Factor Xa, thrombin, and enterokinase.

In another embodiment, two or more copies of the aggrecanase(s) of the present invention are included in the same protein. Such a fusion protein may have enhanced aggrecanase activity.

The truncated ADAMTS proteins can also be tagged with a small epitope and subsequently identified or purified using a specific antibody to the epitope. A preferred epitope is the FLAG™ epitope, which is commercially available from Eastman Kodak (New Haven, Conn.). In addition, the truncated ADAMTS proteins can be expressed as 6×His-tagged proteins for purification using metal chelate affinity chromatography. Materials and methods for His-tagged protein expression and purification are commercially available in kit form (e.g., QIAexpress® system, Qiagen, Valencia, Calif.).

The truncated ADAMTS proteins may also be produced by known conventional chemical synthesis. Methods for chemically synthesizing polypeptides are well known to those skilled in the art. Such chemically synthetic polypeptides may possess biological properties in common with natural, purified polypeptides, and thus may be employed as biologically active or immunological substitutes for natural polypeptides.

Antibody molecules to ADAMTS proteins (particularly aggrecanases) are commercially available from, e.g., Cedarlane Laboratories, Ontario, Canada; Triple Point Biologics, Forest Grove, Oreg.; and Acris GmbH, Hiddenhausen, Germany. Such antibodies should recognize the truncated ADAMTS proteins of the present invention provided they were made to the mature N-terminus (nontruncated portion) of the proteins. Alternatively, antibodies that specifically recognize the truncated ADAMTS proteins of the present invention may be produced by methods well known to those skilled in the art.

For example, polyclonal sera and antibodies can be produced by immunizing a suitable subject with a truncated ADAMTS protein. The antibody titer in the immunized subject may be monitored over time by standard techniques, such as with an enzyme-linked immunosorbent assay (ELISA) using immobilized marker protein. If desired, the antibody molecules may be isolated from the subject or culture media and further purified by well-known techniques, such as protein-A or -G chromatography, to obtain an IgG fraction.

Monoclonal antibodies that recognize a truncated ADAMTS protein can be produced by generation of hybridomas in accordance with known methods. Hybridomas formed in this manner are then screened using standard methods, such as ELISA, to identify one or more hybridomas that produce an antibody that specifically recognizes the protein. The entire truncated ADAMTS protein may be used as the immunogen, or, alternatively, antigenic peptide fragments of the protein may be used. In addition, recombinant monospecific antibodies to the truncated ADAMTS proteins of the present invention can be produced using kits and methods well known to those skilled in the art.

Once the protein is purified, it can be analyzed and verified using standard techniques such as SDS-PAGE or immunoblots. SDS-PAGE can be stained with coommassie blue, silver, or other suitable agents to visualize the purified protein. The purified protein can be further analyzed by protein sequencing or mass spectroscopy. In one example, the protein band of interest is excised manually from an SDS-PAGE, and then reduced, alkylated and digested with trypsin or endopeptidase Lys-C (Promega, Madison, Wis.). The digestion can be conducted in situ using an automated in-gel digestion robot. After digestion, the peptide extracts can be concentrated and separated by microelectrospray reversed phase HPLC. Peptide analyses can be done on a Finnigan LCQ ion trap mass spectrometer (ThermoQuest, San Jose, Calif.). Automated analysis of MS/MS data can be performed using the SEQUEST computer algorithm incorporated into the Finnigan Bioworks data analysis package (ThermoQuest, San Jose, Calif.).

The purified aggrecanase protein can also be analyzed or verified using immunoblots such as Western blot. In one embodiment, protein samples in an SDS-PAGE are transferred to a nitrocellulose membrane and then detected by antibodies. In one example, the purified aggrecanase is detected using a rabbit antibody against the modified ADAMTS, followed by goat-anti-rabbit IgG-HRP and a chemiluminescent substrate (Pierce, Milwaukee, Wis.).

In yet another embodiment, the aggrecanase is expressed using cell-free transcription and translation systems. Suitable cell-free expression systems include, but are not limited to, wheat germ extracts, reticulocyte lysates, or HeLa nuclear extracts.

The truncated ADAMTS of the present invention preferably have aggrecanase activity. Numerous assays are available for detection of the biological activities of a truncated ADAMTS of the present invention. Exemplary assays include, but are not limited to, the fluorescent peptide assay, the neoepitope Western blot, the aggrecan ELISA, and the activity assay. The first two assays are suitable for detecting the cleavage capability at the Glu³⁷³-Ala³⁷⁴ bond in the IGD of aggrecan.

In the fluorescent peptide assay, the aggrecanase is incubated with a synthetic peptide which contains the amino acid sequence at the aggrecanase cleavage site. Either the N-terminus or the C-terminus of the synthetic peptide is labeled with a fluorophore and the other terminus includes a quencher. Cleavage of the peptide separates the fluorophore and quencher, thereby eliciting fluorescence. Relative fluorescence can be used to determine the relative activity of the expressed aggrecanase.

In the neoepitope Western blot, the aggrecanase is incubated with intact aggrecan. The cleavage products are then subject to several biochemical treatments before being separated by an SDS-PAGE. The biochemical treatments include, for example, dialysis, chondroitinase treatment, lyophilization, and reconstitution. Protein samples in the SDS-PAGE are transferred to a membrane (such as a nitrocellulose paper), and stained with a neoepitope specific antibody. The neoepitope antibody specifically recognizes a new N- or C-terminal amino acid sequence exposed by proteolytic cleavage of aggrecan. The antibody does not bind to such an epitope on the original or uncleaved molecule. Suitable neoepitope specific antibodies include, but are not limited to, MAb BC-13, MAb BC-3, and the 119C antibody. See, e.g., Caterson, et al., supra; and Hashimoto, et al., FEBS LETTERS, 494:192-195 (2001). Cleaved aggrecan fragments can be visualized using an alkaline phosphatases-conjugated secondary antibody and nitroblue tetrazolium chromogen and bromochloroindolyl phosphate substrate (NBT/BCIP). Relative density of the bands is indicative of relative aggrecanase activity.

The aggrecan ELISA can be used to detect any cleavage in an aggrecan molecule. In this assay, the modified protein is incubated with intact aggrecan which has been previously adhered to plastic wells. The wells are washed and then incubated with an antibody that detects aggrecan. The wells are developed with a secondary antibody. If the original amount of aggrecan remains in the wells, the antibody staining would be dense. If aggrecan is digested by the aggrecanase, the attached aggrecan molecule will come off the wells, thereby reducing the subsequent staining by the antibody. This assay can detect whether a modified protein is capable of cleaving aggrecan. The relative cleavage activity of the modified protein can also be determined using this assay.

The activity assay can also be employed to assess the cleavage activity of the aggrecanase. In this assay, microtiter plates are first coated with hyaluronic acid (ICN), followed by chondroitinase-treated bovine aggrecan. Chondroitinase can be obtained from Seikagaku Chemicals. The culture medium containing the expressed recombinant aggrecanase is added to the aggrecan-coated plates. Aggrecan cleaved at the Glu³⁷³-Ala³⁷⁴ within the IGD is washed away. The remaining uncleaved aggrecan can be detected with the 3B3 antibody (ICN), followed by anti-IgM-HRP secondary antibody (Southern Biotechnology). Final color development can be obtained using, for example, 3,3″, 5,5″ tetramethylbenzidine (TMB, BioFx Laboratories).

In many embodiments, the aggrecanases of the present invention have improved stability and increased expression. This allows the isolation of an aggrecanase in large amounts, thereby facilitating the development of aggrecanase inhibitors.

Inhibitors can be developed using any suitable screen assay. Typically, a screen method involves contacting the aggrecanase with an aggrecanase substrate in the presence or absence of a compound of interest. The cleavage activity of the aggrecanase is then measured to determine the inhibitory effect of the compound of interest. See, e.g., Hashimoto, et al., supra. In one embodiment, inhibitors are screened using high throughput processes or compound libraries. Following their expression and purification, the truncated biologically active ADAMTS proteins may be used in screening assays to identify pharmacological agents or lead compounds capable of modulating ADAMTS activity. For example, samples containing purified truncated ADAMTS protein can be contacted with one of a plurality of test compounds (e.g., small organic molecules, biological agents), and the activity of the ADAMTS protein (e.g., hyelectanase activity, aggrecanase activity, α₂-macroglobulin cleavage activity) compared to the activity of uncontacted protein or protein contacted with a different test compound(s) to determine whether any of the test compounds provides 1) a substantially decreased level of ADAMTS activity, thereby indicating an inhibitor of ADAMTS activity; or 2) a substantially increased level of ADAMTS activity, thereby indicating an activator of ADAMTS activity.

Preferably, the purified truncated ADAMTS proteins possess hyelectanase activity and more preferably, aggrecan-cleaving activity, and are used in the above-mentioned screening assays to identify inhibitors of hyelectanase and/or aggrecanase activity. Several selective aggrecanase inhibitors have been identified using similar screening assays (see, e.g., Cherney et al., Bioorg. Med. Chem. Lett. 12:101 (2002); Yao et al., Bioorg. Med. Chem. Lett. 13:1297 (2003); Yao et al., J. Med. Chem. 44:3347 (2001)). Assays for aggrecanase activity are well known in the art and include the aggrecan-polyacrylamide particle assay (Vankemmelbeke et al., Eur. J. Biochem. 270:2394 (2003)) and detection of aggrecan core protein fragments by SDS-PAGE (Hashimoto et al., FEBS Lett. 494:192 (2001)).

Preferably, the aggrecanase activity assay described above is an immunoassay. Such immunoassays utilize an antibody that specifically recognizes an aggrecan neoepitope produced by the enzymatic activity of a truncated ADAMTS protein (preferably at the Glu³⁷³-Ala³⁷⁴ position in aggrecan). Such antibodies, for example BC-3 (which recognizes N-terminal neoepitope ³⁷⁴ARGSV) and BC-13 (which recognizes the C-terminal neoepitope ITEGE³⁷³), are well known in the art (Hughes et al., Biochem. J. 305:799 (1995)) or can be produced by methods well known to those skilled in the art, and can be used to detect aggrecan cleavage products by Western blot and ELISA (see, e.g., Miller et al., Anal. Biochem. 314:260 (2003); Hughes et al., J. Biol. Chem. 272:20269 (1997)).

Compounds identified by the screening assays described above (particularly those that inhibit aggrecanase activity) can be formulated according to methods known in the art and administered in vivo in the form of pharmaceutical compositions for the treatment of arthritis and other inflammatory disorders. The pharmaceutical compositions may be administered by any number of routes that are well known in the art, including, but not limited to, intraarticular, oral, nasal, rectal, topical, sublingual, intravenous, intramuscular, intraarterial, intramedullary, intrathecal, intraventricular, intraperitoneal, and transdermal routes. In addition to the active ingredients, the pharmaceutical compositions may contain pharmaceutically acceptable carriers comprising, for example, excipients, coatings, and auxiliaries well known in the art.

Inhibitors can also be identified or designed using three-dimensional structural analysis or computer aided drug design. The latter method may entail determination of binding sites for inhibitors based on the three dimensional structure of aggrecanase or aggrecan, and then developing molecules reactive with the binding site(s) on aggrecanase or aggrecan. Candidate molecules are subsequently assayed for inhibitory activity. Other conventional methods suitable for developing protease inhibitors can also be employed to identify aggrecanase inhibitors.

Aggrecanase inhibitors can be, for example, proteins, peptides, antibodies, small molecules, or chemical compounds. An inhibitor can produce a reduction, a diminution, or an elimination of the proteolytic activity of an aggrecanase. The reduction, diminution, or elimination of aggrecanase activity can be measured by the assays described above. In one example, an inhibitor of the present invention can reduce aggrecanase activity by at least 10%, 20%, 30%, 40%, 50%, 60%, 70%, 80% 90%, or more. In another example, the aggrecanase inhibitor specifically reduces or eliminates the enzymatic activity of aggrecanases but not other proteases, such as MMPs. In yet another example, the aggrecanase inhibitor reduces or eliminates the aggrecanase activity of specific ADAMTS protein(s), but not other ADAMTS protein(s).

Various diseases or conditions are characterized by degradation of aggrecan. Aggrecanase inhibitors identified by the present invention can be used in the treatment of these diseases or conditions. Diseases that are contemplated as being treatable by using aggrecanase inhibitors include, but are not limited to, osteoarthritis, cancer, inflammatory joint disease, rheumatoid arthritis, septic arthritis, periodontal diseases, corneal ulceration, proteinuria, coronary thrombosis from atherosclerotic plaque rupture, aneurysmal aortic disease, inflammatory bowel disease, Crohn's disease, emphysema, acute respiratory distress syndrome, asthma, chronic obstructive pulmonary disease, Alzheimer's disease, brain and hematopoietic malignancies, osteoporosis, Parkinson's disease, migraine, depression, peripheral neuropathy, Huntington's disease, multiple sclerosis, ocular angiogenesis, macular degeneration, aortic aneurysm myocardial infarction, autoimmune disorders, degenerative cartilage loss following traumatic joint injury, head trauma, dystrophobic epidermolysis bullosa, spinal cord injury, acute and chronic neurodegenerative diseases, osteopenias, tempero mandibular joint disease, demyelating diseases of the nervous system, organ transplant toxicity and rejection, cachexia, allergy, tissue ulcerations, restenosis, and other diseases characterized by abnormal degradation of the extracellular matrix, altered aggrecanase activity, or altered aggrecanase level.

As used herein, treatment includes therapeutic treatment or prophylactic or preventative measures. Those in need of treatment can include individuals already having a particular medical disorder as well as those who may ultimately acquire the disorder (i.e., those needing preventative measures). Treatment may regulate aggrecanase activity or the protein level of aggrecanase to prevent or ameliorate clinical symptoms of the disease. The inhibitors can function by, for example, preventing the interaction between aggrecanase and aggrecan, or reducing or eliminating the proteolytic activity.

In one embodiment, the aggrecanase inhibitor of the present invention is administered to a patient or animal in a pharmaceutical composition. The pharmaceutical composition includes an effective amount of the inhibitor that is sufficient to treat the patient or animal. The pharmaceutical composition can also include a pharmaceutically acceptable carrier. The pharmaceutically acceptable carrier can include solvents, solubilizers, fillers, stabilizers, binders, absorbents, bases, buffering agents, lubricants, controlled release vehicles, diluents, emulsifying agents, humectants, lubricants, dispersion media, coatings, antibacterial or antifungal agents, isotonic and absorption delaying agents, and the like, that are compatible with pharmaceutical administration. The use of such media and agents for pharmaceutically active substances is well-known in the art. Supplementary agents can also be incorporated into the composition.

The pharmaceutical composition can be formulated to be compatible with its intended route of administration. Examples of routes of administration include parenteral, intravenous, intradermal, subcutaneous, oral, inhalation, transdermal, rectal, transmucosal, topical, and systemic administration. In one example, the administration is carried out by using an implant.

Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates; and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

The pharmaceutical composition can be administered to the patient or animal so that the aggrecanase inhibitor is in a sufficient amount to reduce or abolish the targeted aggrecanase activity. Suitable therapeutic dosages for an aggrecanase inhibitor can range, for example, from 5 mg to 100 mg, from 15 mg to 85 mg, from 30 mg to 70 mg, or from 40 mg to 60 mg. Dosages below 5 mg or above 100 mg can also be used. Inhibitors can be administered in one dose or multiple doses. The doses can be administered at intervals such as once daily, once weekly, or once monthly. Dosage schedules for administration of an aggrecanase inhibitor can be adjusted based on, for example, the affinity of the inhibitor for its aggrecanase target, the half-life of the inhibitor, and the severity of the patient's condition. In one embodiment, inhibitors are administered as a bolus dose, to maximize their circulating levels. In another embodiment, continuous infusions are used after the bolus dose.

Toxicity and therapeutic efficacy of aggrecanase compounds can be determined by standard pharmaceutical procedures in cell culture or experimental animal models. For instance, the LD₅₀ (the dose lethal to 50% of the population) and the ED₅₀ (the dose therapeutically effective in 50% of the population) can be determined. The dose ratio between toxic and therapeutic effects is the therapeutic index, and can be expressed as the ratio LD₅₀/ED₅₀. In one example, inhibitors which exhibit large therapeutic indices are selected.

The data obtained from cell culture assays and animal studies can be used in formulating a range of dosages for use in humans. The dosage of such compounds may lie within a range of circulating concentrations that exhibit an ED₅₀ with little or no toxicity. The dosage may vary within this range depending upon the dosage form employed and the route of administration utilized. For any inhibitor used according to the present invention, a therapeutically effective dose can be estimated initially from cell culture assays. A dose may be formulated in animal models to achieve a circulating plasma concentration range that exhibits an IC₅₀ (i.e., the concentration of the test inhibitor which achieves a half-maximal inhibition of symptoms) as determined by cell culture assays. Levels in plasma may be measured, for example, by high performance liquid chromatography. The effects of any particular dosage can be monitored by suitable bioassays. Examples of suitable bioassays include DNA replication assays, transcription-based assays, GDF protein/receptor binding assays, creatine kinase assays, assays based on the differentiation of pre-adipocytes, assays based on glucose uptake in adipocytes, and immunological assays.

The dosage regimen for the administration of composition can be determined by the attending physician based on various factors which modify the action of the aggrecanase protein, the site of pathology, the severity of disease, the patient's age, sex, and diet, the severity of any inflammation, time of administration and other clinical factors. Generally, systemic or injectable administration will be initiated at a dose which is minimally effective, and the dose will be increased over a preselected time course until a positive effect is observed. Subsequently, incremental increases in dosage will be made limiting to levels that produce a corresponding increase in effect while taking into account any adverse affects that may appear. The addition of other known factors to a final composition may also affect the dosage.

Progress can be monitored by periodic assessment of disease progression. The progress can be monitored, for example, by X-rays, MRI or other imaging modalities, synovial fluid analysis, or clinical examination.

Where a disease is caused by accumulation of aggrecan or other extracellular matrix proteins, an aggrecanase of the present invention can be introduced into a human or animal affected by the disease to correct such deficiency. The aggrecanase thus introduced should be proteolytically active against the extracellular matrix protein at issue. Methods for administering a therapeutic protein to a human or animal are well known in the art. Suitable methods include those described above. In addition, a gene therapy-based approach can be employed.

The aggrecanase inhibitor of the present invention can be used in assays and methods of detection to determine the presence or absence of, or quantify aggrecanase in a sample. The assays or methods of detection can be in vivo or in vitro. By correlating the presence or level of these proteins with a disease, one of skill in the art can diagnose the associated disease or determine its severity. Diseases that may be diagnosed by the presently disclosed inhibitors are set forth above.

Where inhibitors are intended for diagnostic purposes, it may be desirable to modify them; for example, with a ligand group (such as biotin or other molecules having specific binding partners) or a detectable marker group (such as a fluorophore, a chromophore, a radioactive atom, an electron-dense reagent, or an enzyme). Molecules having specific binding partners include, for example, biotin and avidin or streptavidin, IgG and protein A, and numerous receptor-ligand couples known in the art. Enzymes are typically detected by their activity. For example, horseradish peroxidase can be detected by its ability to convert tetramethylbenzidine (TMB) to a blue pigment, quantifiable with a spectrophotometer.

It should be understood that the above-described embodiments and the following examples are given by way of illustration, not limitation. Various changes and modifications within the scope of the present invention will become apparent to those skilled in the art from the present description.

### EXAMPLES

### Example 1

### Construction of Truncated ADAMTS

The overall domain structures of representative full-length ADAMTS-7, -9, -10, -16 and -18 proteins are depicted in FIG. 1. Like other full-length ADAMTS family members, ADAMTS-7, -9, -10, -16 and -18 have a signal peptide (SP), a pro peptide (Pro), a catalytic domain (Cat domain), a disintegrin-like domain (Disint), a thrombospondin type 1 repeat (Tsp), a cysteine-rich domain (Cys-rich), a spacer domain (Spacer), and a variable number of carboxy-terminus thrombospondin repeats (T). ADAMTS-7 further contains one additional spacer domain located between the third and fourth carboxyl terminal thrombospondin repeats. A spatially conserved phenylalanine residue after the central thrombospondin type I repeat, Phe⁵⁹⁹ for ADAMTS-7, Phe⁶⁴⁹ for ADAMTS-9, Phe⁶⁰⁸ for ADAMTS-10, Phe⁶⁴⁷ for ADAMTS-16, and Phe⁶⁵⁰ for ADAMTS-18 is indicated in FIG. 1.

The domain structures of five truncated ADAMTS-7 (A7FS), ADAMTS-9 (A9FS), ADAMTS-10 (A10FS), ADAMTS-16 (A16FS), and ADAMTS-18 (A18FS) proteins are illustrated in FIG. 2. Each truncation includes deletion of all of the amino acid residues that are located C-terminal to the conserved phenylalanine residue. A Step-tag is added to the C-terminus of each truncated ADAMTS to aid protein purification. The amino acid sequences for A7FS, A9FS, A10FS, A16FS, and A18FS are depicted in SEQ ID NOs:41-45, respectively.

The DNA coding sequences for A7FS, A9FS, A10FS, A16FS, and A18FS can be prepared using PCR. PCR primers can be designed from the published sequences of human ADAMTS-7 (GenBank Accession No. AF140675), ADAMTS-9 (GenBank Accession No. AF261918), ADAMTS-10 (GenBank Accession No. NP_{—}112219), ADAMTS-16 (GenBank Accession No. NP_{—}620687) and ADAMTS-18 (GenBank Accession No. NP_{—}955387). In one example, the A7FS or A9FS coding sequence can be amplified from a suitable human cDNA library (e.g., a heart, skeletal muscle, kidney, or pancreas cDNA library) using the Advantage-GC PCR kit (Clontech). Reaction conditions can be those recommended by the manufacturer. In certain cases, the reaction conditions include the following exceptions: the amount of GC Melt used is 10 μl per 50 μl reaction; the amount of Not I linearized library used is 0.2 ng/μl reaction; and the amount of each oligo used is 2 pmol/μl reaction. Cycling conditions are as follows: 95° C. for 1 min, one cycle; followed by 30 cycles consisting of 95° C. for 15 sec/68° C. for 2 min.

The 5′ primer for the PCR amplification can incorporate an EcoR I site (GAATTC) and a modified Kozak sequence (CCACC) upstream of the start codon (ATG) of the ADAMTS-7, -9, -10, -16, and -18 coding sequence. The 3′ primer for the PRC amplification can incorporate an additional sequence encoding the linker “GSA,” the Step-tag, a stop codon (e.g., TAA), and a Not I site (GCGGCCGC). The additional sequence can be added downstream of the codon for the conserved phenylalanine residue. PCR products with the appropriate sizes are isolated, and then digested with EcoR I and Not I. The digested products are ligated into an expression vector which includes the same restriction sites. The cloned PCR fragments can be sequenced to verify their identities.

In one example, the expression vector is a CHO cell expression vector, such as the pTmed vector, the sequence of which is shown in SEQ ID NO:8.

### Example 2

### Expression and Purification of Truncated ADAMTS

The pTmed vector containing the A7FS, A9FS, A10FS, A16FS, or A18FS sequence was transfected into CHO/DUKX cells using the manufacturer's recommended protocol for lipofection (Lipofectin from InVitrogen). Clones were selected in 0.02 μM methotrexate. Colonies were picked and expanded into cell lines while cultured in selection medium.

Cell lines expressing the highest level of recombinant protein were selected by monitoring recombinant protein in CHO conditioned media by Western blotting using an anti-streptavidin antibody conjugated to horseradish peroxidase (HRP) (Southern Biotech) followed by ECL chemiluminescence (Amersham Biosciences) and autoradiography.

Recombinant proteins were purified by a combination of ultrafiltration and affinity purification on a Strep-Tactin column (IBA). CHO condition media was concentrated approximately 35-fold by ultra-filtration utilizing a 10,000 MWCO filter. The condition media retentate was then applied to a Strep-Tactin affinity column. Non-specifically bound proteins were removed from the column by application of multiple aliquotes of wash buffer following the manufacturers recommended protocol. Recombinant protein was eluted from the column by the addition of desthiobiotin.

### Example 3

### Detection of Aggrecanase Activity of A7FS, A9FS, A10FS, A16FS and A18FS

Aggrecanase activity was assayed by incubating bovine aggrecan with purified recombinant protein followed by SDS-PAGE fractionation and Western blot analysis of the digest. Western blots were probed with C1 monoclonal antibody (C1 MAb), which specifically recognizes a neoepitope generated by the proteolysis of aggrecan (i.e., the carboxyl terminal sequence . . . NITEGE³⁷³ (SEQ ID NO:9) of the ˜70 kDa G1-bearing product after cleavage of aggrecan at the Glu³⁷³-Ala³⁷⁴ bond). C1 MAb was visualized by incubation with NBT/BCIP substrate (Promega).

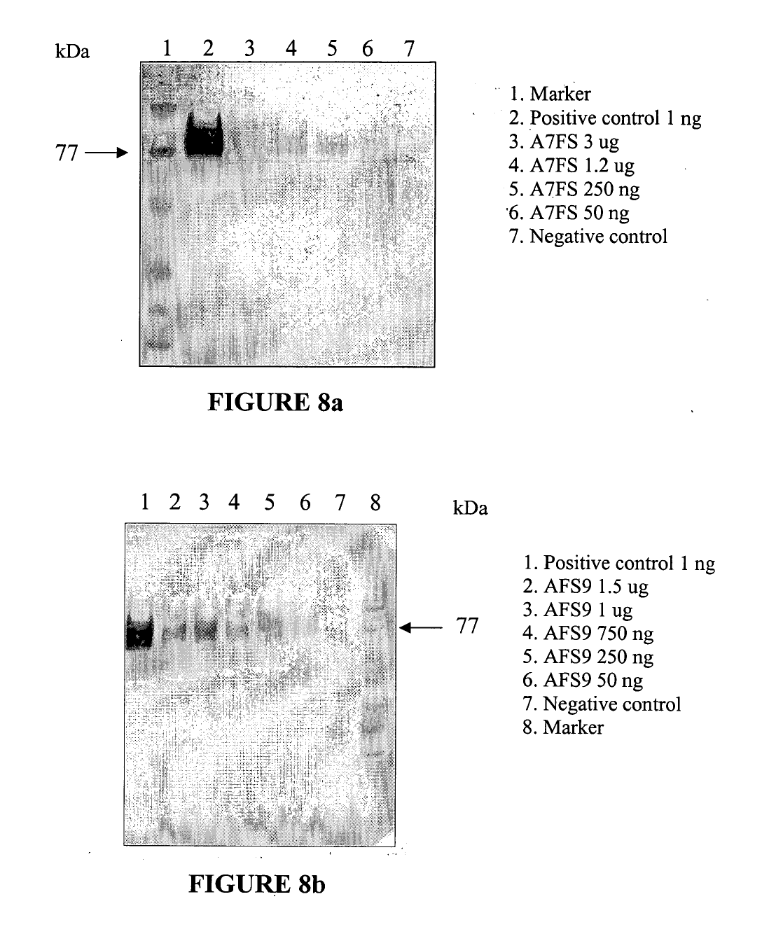

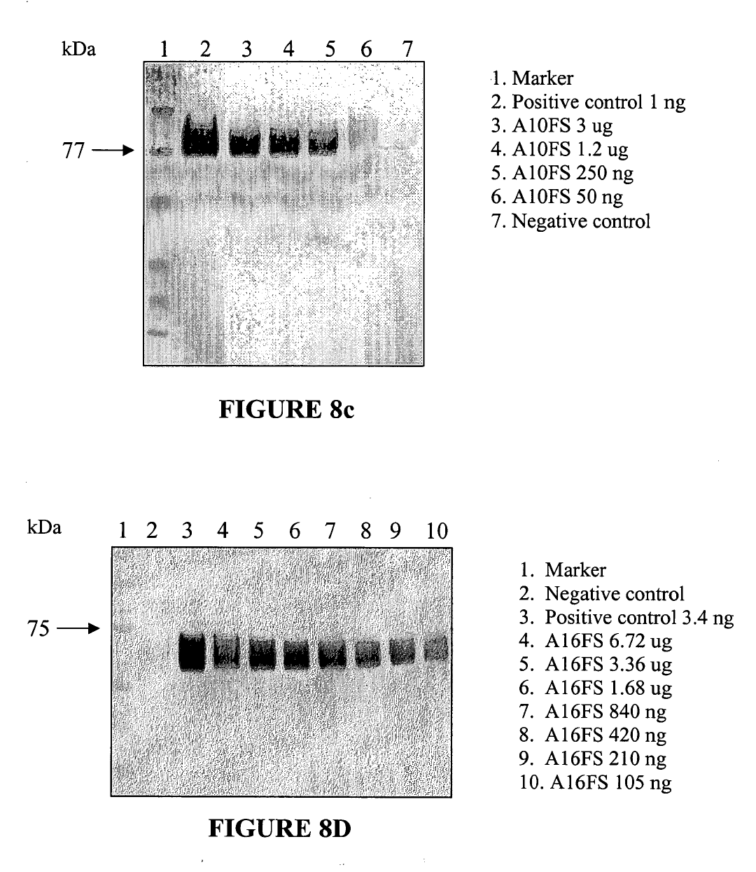

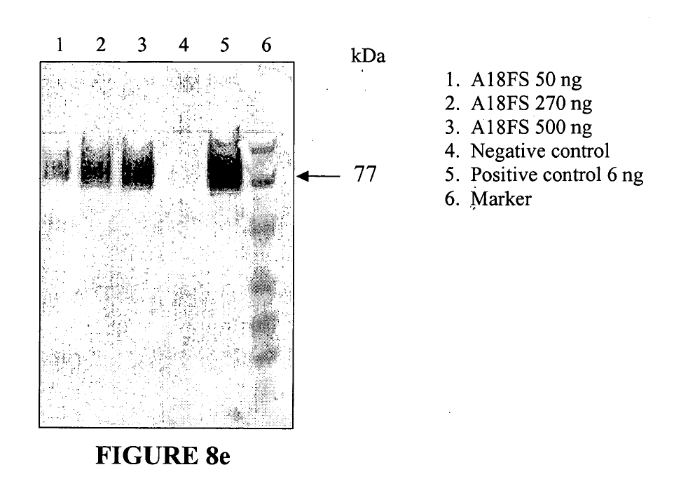

FIGS. 8A-8E show bovine aggrecan digestion with recombinant A7FS protein, A9FS protein, A10FS protein, A16FS, and A18FS protein, respectively. Digested protein was fractionated on SDS-PAGE then transferred to a nylon membrane for Western blot analysis. Negative control is bovine aggrecan minus recombinant protein. Positive control is recombinant aggrecanase 1 protein (ADAMTS-4).

### Example 4

### Production of C1 MAb

The synthetic peptide CGGPLPRNITEGE (SEQ ID NO:46) was coupled to the carrier protein KLH, and the conjugate was used as the immunogen for the production of monoclonal antibodies by standard hybridoma technology. Briefly, BALB/c mice were immunized subcutaneously with 20 μg of immunogen in complete Freund's adjuvant. The injection was repeated twice (biweekly) using peptide in incomplete Freund's adjuvant. Test bleeds were done on the immunized mice, and serum was evaluated by ELISA for reactivity against both the immunizing peptide and ADAMTS-4-digested bovine articular cartilage aggrecan (Flannery et al., supra). Three days prior to hybridoma fusion, a final immunization without adjuvant was given to the mouse exhibiting highest antibody titer. Spleen cells from this mouse were isolated and fused with FO myeloma cells (American Type Culture Collection, Manassas, Va.) and cultured in HAT selection medium (Sigma-Aldrich, St. Louis, Mo.). Hybridoma culture supernatants were screened against KLH and KLH-CGGPLPRNITEGE antigens by ELISA, and against ADAMTS-4-digested aggrecan by Western blotting. Positive hybridoma clones were selected for subcloning by limiting dilution. A single hybridoma cell line, designated C1 MAb, was expanded in culture. Antibody isotype was determined to be IgG1 (κ light chain) using the Mouse Monoclonal Antibody Isotyping kit (Roche, Indianapolis, Ind.) and IgG from 1 liter of culture media was purified by Protein A affinity chromatography.

### Example 5

### Expression Vectors

The mammalian expression vector pMT2 CXM, which is a derivative of p91023(b), can also be used in the present invention. The pMT2 CXM vector differs from p91023(b) in that the former contains the ampicillin resistance gene in place of the tetracycline resistance gene and further contains an Xho I site for insertion of cDNA clones. The functional elements of pMT2 CXM include the adenovirus VA genes, the SV40 origin of replication (including the 72 bp enhancer), the adenovirus major late promoter (including a 5′ splice site and the majority of the adenovirus tripartite leader sequence present on adenovirus late mRNAs), a 3′ splice acceptor site, a DHFR insert, the SV40 early polyadenylation site (SV40), and pBR322 sequences needed for propagation in E. coli

Plasmid pMT2 CXM is obtained by EcoR I digestion of pMT2-VWF, which has been deposited with the American Type Culture Collection (ATCC), Rockville, Md. (USA) under accession number ATCC 67122. EcoR I digestion excises the cDNA insert present in pMT2-VWF, yielding pMT2 in linear form which can be ligated and used to transform E. coli HB 101 or DH-5 to ampicillin resistance. Plasmid pMT2 DNA can be prepared by conventional methods. pMT2 CXM is then constructed using loopout/in mutagenesis. This removes bases 1075 to 1145 relative to the Hind III site near the SV40 origin of replication and enhancer sequences of pMT2. In addition, it inserts a sequence containing the recognition site for the restriction endonuclease Xho I. A derivative of pMT2CXM, termed pMT23, contains recognition sites for the restriction endonucleases Pst I, EcoR I, Sal I and Xho I. Plasmid pMT2 CXM and pMT23 DNA may be prepared by conventional methods.

pEMC2B1 derived from pMT21 may also be suitable in practice of the present invention. pMT21 is derived from pMT2 which is derived from pMT2-VWF. As described above, EcoR I digestion excises the cDNA insert present in pMT-VWF, yielding pMT2 in linear form which can be ligated and used to transform E. Coli HR 101 or DH-5 to ampicillin resistance. Plasmid pMT2 DNA can be prepared by conventional methods.

pMT21 is derived from pMT2 through the following two modifications. First, 76 bp of the 5′ untranslated region of the DHFR cDNA including a stretch of 19 G residues from G/C tailing for cDNA cloning is deleted. In this process, Pst I, EcoR I, and Xho I sites are inserted immediately upstream of DHFR.

Second, a unique Cla I site is introduced by digestion with EcoR V and Xba I, treatment with Klenow fragment of DNA polymerase I, and ligation to a Cla I linker (CATCGATG). This deletes a 250 bp segment from the adenovirus associated RNA (VAI) region but does not interfere with VAI RNA gene expression or function. pMT21 is digested with EcoR I and Xho I, and used to derive the vector pEMC2B1.

A portion of the EMCV leader is obtained from pMT2-ECAT1 by digestion with EcoR I and Pst I, resulting in a 2752 bp fragment. This fragment is digested with Taq I yielding an EcoR I-Taq I fragment of 508 bp which is purified by electrophoresis on low melting agarose gel. A 68 bp adapter and its complementary strand are synthesized with a 5′ Taq I protruding end and a 3′ Xho I protruding end.

The adapter sequence matches the EMC virus leader sequence from nucleotide 763 to 827. It also changes the ATG at position 10 within the EMC virus leader to an ATT and is followed by an Xho I site. A three way ligation of the pMT21 EcoR I-Xho I fragment, the EMC virus EcoR I-Taq I fragment, and the 68 bp oligonucleotide adapter Taq I-Xho I adapter resulting in the vector pEMC2β1.

This vector contains the SV40 origin of replication and enhancer, the adenovirus major late promoter, a cDNA copy of the majority of the adenovirus tripartite leader sequence, a small hybrid intervening sequence, an SV40 polyadenylation signal and the adenovirus VA I gene, DHFR and β-lactamase markers and an EMC sequence, in appropriate relationships to direct the high level expression of the desired cDNA in mammalian cells.

The construction of vectors may involve modification of the aggrecanase-related DNA sequences. For instance, a cDNA encoding an aggrecanase can be modified by removing the non-coding nucleotides on the 5′ and 3′ ends of the coding region. The deleted non-coding nucleotides may or may not be replaced by other sequences known to be beneficial for expression. These vectors are transformed into appropriate host cells for expression of an aggrecanase of the present invention.

In one example, the mammalian regulatory sequences flanking the coding sequence of aggrecanase are eliminated or replaced with bacterial sequences to create bacterial vectors for intracellular or extracellular expression of the aggrecanase molecule. The coding sequences can be further manipulated (e.g. ligated to other known linkers or modified by deleting non-coding sequences therefrom or altering nucleotides therein by other known techniques). An aggrecanase encoding sequence can then be inserted into a known bacterial vector using procedures as appreciated by those skilled in the art. The bacterial vector can be transformed into bacterial host cells to express the aggrecanases of the present invention. For a strategy for producing extracellular expression of aggrecanase proteins in bacterial cells, see, e.g. European Patent Application 177,343.

Similar manipulations can be performed for construction of an insect vector for expression in insect cells (see, e.g., procedures described in published European Patent Application 155,476). A yeast vector can also be constructed employing yeast regulatory sequences for intracellular or extracellular expression of the proteins of the present invention in yeast cells (see, e.g., procedures described in published PCT application WO86/00639 and European Patent Application 123,289).

A method for producing high levels of aggrecanase proteins in mammalian, bacterial, yeast, or insect host cell systems can involve the construction of cells containing multiple copies of the heterologous aggrecanase gene. The heterologous gene can be linked to an amplifiable marker, e.g., the dihydrofolate reductase (DHFR) gene for which cells containing increased gene copies can be selected for propagation in increasing concentrations of methotrexate (MTX). This approach can be employed with a number of different cell types.

For example, a plasmid containing a DNA sequence for an aggrecanase in operative association with other plasmid sequences enabling expression thereof and an DHFR expression plasmid (such as, pAdA26SV(A)3) can be co-introduced into DHFR-deficient CHO cells (DUKX-BII) by various methods including calcium phosphate-mediated transfection, electroporation, or protoplast fusion. DHFR expressing transformants are selected for growth in alpha media with dialyzed fetal calf serum, and subsequently selected for amplification by growth in increasing concentrations of MTX (e.g. sequential steps in 0.02, 0.2, 1.0 and 5 μM MTX). Transformants are cloned, and biologically active aggrecanase expression is monitored by at least one of the assays described above. Aggrecanase protein expression should increase with increasing levels of MTX resistance. Aggrecanase polypeptides are characterized using standard techniques known in the art such as pulse labeling with ³⁵S methionine or cysteine and polyacrylamide gel electrophoresis. Similar procedures can be followed to produce other aggrecanases.

### Example 6

### Transfection of Expression Vectors

As one example, an aggrecanase nucleotide sequence of the present invention is cloned into the expression vector pED6. COS and CHO DUKX B11 cells (Urlaub and Chasin, PROC. NATL. ACAD. SCI. USA, 77:4218-4220 (1980)) are transiently transfected with the aggrecanase sequence by lipofection (LF2000, Invitrogen) (+/−co-transfection of PACE on a separate PED6 plasmid). Duplicate transfections are performed for each molecule of interest: (a) one transfection set for harvesting conditioned media for activity assay and (b) the other transfection set for 35-S-methionine/cysteine metabolic labeling.

On day one, media is changed to DME(COS) or alpha (CHO) media plus 1% heat-inactivated fetal calf serum +/−100 μg/ml heparin on wells of set (a) to be harvested for activity assay. After 48 h, conditioned media is harvested for activity assay.

On day 3, the duplicate wells of set (b) are changed to MEM (methionine-free/cysteine free) media plus 1% heat-inactivated fetal calf serum, 100 μg/ml heparin and 100 μCi/ml ³⁵S-methionine/cysteine (Redivue Pro mix, Amersham). Following 6 h incubation at 37° C., conditioned media is harvested and run on SDS-PAGE gels under reducing conditions. Proteins can be visualized by autoradiography.

The foregoing description of the present invention provides illustration and description, but is not intended to be exhaustive or to limit the invention to the precise one disclosed. Modifications and variations are possible consistent with the above teachings or may be acquired from practice of the invention. Thus, it is noted that the scope of the invention is defined by the claims and their equivalents.

1. An isolated or recombinant aggrecanase obtainable by deleting from a full-length ADAMTS protein a plurality of amino acid residues, wherein the full-length ADAMTS protein comprises a cysteine-rich domain, and said plurality of deleted amino acid residues comprise a substantial portion of the cysteine-rich domain, and wherein the full-length ADAMTS protein is not a full-length ADAMTS-4 protein.

2. The aggrecanase according to claim 1, wherein the full-length ADAMTS protein comprises a thrombospondin type I repeat located N-terminal to the cysteine-rich domain, and a conserved phenylalanine residue located C-terminal to the thrombospondin type I repeat, and wherein said plurality of deleted amino acid residues comprise a substantial portion of all of the amino acid residues that are located C-terminal to the conserved phenylalanine residue.

3. The aggrecanase according to claim 2, wherein the conserved phenylalanine residue is the first conserved phenylalanine residue that is located C-terminal to the thrombospondin type I repeat.

4. The aggrecanase according to claim 3, wherein said plurality of deleted amino acid residues comprise all of the amino acid residues that are located C-terminal to the conserved phenylalanine residue.

5. The aggrecanase according to claim 1, further comprising a deletion of a substantial portion of the prodomain.

6. The aggrecanase according to claim 1, wherein the full-length ADAMTS protein is selected from the group consisting of ADAMTS-7, ADAMTS-9, ADAMTS-10, ADAMTS-16 and ADAMTS-18.

7. The aggrecanase according to claim 1, consisting of an amino acid sequence selected from the group consisting of SEQ ID NO:2, SEQ ID NO:3, SEQ ID NO:4, SEQ ID NO:6, SEQ ID NO:7, SEQ ID NO:8, SEQ ID NO:10, SEQ ID NO:11, SEQ ID NO:12, SEQ ID NO:14, SEQ ID NO:15, SEQ ID NO:16, SEQ ID NO:18, SEQ ID NO:19, and SEQ ID NO:20.

8. The aggrecanase according to claim 9, consisting of a variant of the amino acid sequence.

9. An isolated or recombinant protein comprising the aggrecanase of claim 1 and a polypeptide covalently linked to the aggrecanase.

10. A polynucleotide encoding the aggrecanase of claim 1.

12. A kit or assay system comprising the aggrecanase of claim 1 or a polynucleotide encoding the same.

13. A method of identifying a compound capable of modulating the activity of an aggrecanase comprising the steps of:
(a) contacting a sample containing the truncated aggrecanase of claim 1 with one of a plurality of test compounds; and
(b) comparing the activity of the contacted sample with that of a corresponding protein sample not contacted with a test compound,
wherein a substantial decrease in activity identifies a compound as a modulator of aggrecanase activity.

14. The method according to claim 13, wherein the compound inhibits said aggrecanase activity.

15. The method according to claim 13, wherein the compound increases said aggrecanase activity.

16. An antibody specific for the aggrecanase of claim 1.

17. An isolated or recombinant aggrecanase consisting essentially of a catalytic domain, a disintegrin domain, and a central thrombospondin type 1 repeat of a full-length ADAMTS protein, wherein the full-length ADAMTS is not an ADAMTS-4 protein.

18. A composition comprising a purified truncated aggrecanase of claim 1.

19. A host cell transformed or transfected with the nucleic acid molecule of claim 10.

20. A method of producing purified truncated aggrecanase comprising the steps of:
(a) culturing the host cell of claim 19 under conditions such that said protein is expressed; and
(b) recovering and purifying said protein from the cell or culture medium.

21. A method for the treatment of an inflammatory condition in a subject comprising administering a compound identified by the method of claim 13.


SEQUENCE LISTING (44 sequences)

SEQ ID NO: 1 - protein, 1686 aa, homo sapiens
MPGGPSPRSPAPLLRPLLLLLCALAPGAPGPAPGRATEGRAALDIVHPVRVDAGGSFLSYELWPRALRKRDVSVRRDAPAFYELQYRGRELRFNLTANQHLLAPGFVSETRRRGGLGRAHIRAHTPACHLLGEVQDPELEGGLAAISACDGLKGVFQLSNEDYFIEPLDSAPARPGHAQPHVVYKRQAPERLAQRGDSSAPSTCGVQVYPELESRRERWEQRQQWRRPRLRRLHQRSVSKEKWVETLVVADAKMVEYHGQPQVESYVLTIMNMVAGLFHDPSIGNPIHITIVRLVLLEDEEEDLKITHHADNTLKSFCKWQKSINMKGDAHPLHHDTAILLTRKDLCAAMNRPCETLGLSHVAGMCQPHRSCSINEDTGLPLAFTVAHELGHSFGIQHDGSGNDCEPVGKRPFIMSPQLLYDAAPLTWSRCSRQYITRFLDRGWGLCLDDPPAKDIIDFPSVPPGVLYDVSHQCRLQYGAYSAFCEDMDNVCHTLWCSVGTTCHSKLDAAVDGTRCGENKWCLSGECVPVGFRPEAVDGGWSGWSAWSICSRSCGMGVQSAERQCTQPTPKYKGRYCVGERKRFRLCNLQACPAGRPSFRHVQCSHFDAMLYKGQLHTWVPVVNDVNPCELHCRPANEYFAEKLRDAVVDGTPCYQVRASRDLCINGICKNVGCDFEIDSGAMEDRCGVCHGNGSTCHTVSGTFEEAEGLGYVDVGLIPAGAREIRIQEVAEAANFLALRSEDPEKYFLNGGWTIQWNGDYQVAGTTFTYARRGNWENLTSPGPTKEPVWIQLLFQESNPGVHYEYTIHREAGGHDEVPPPVFSWHYGPWTKCTVTCGRGVQRQNVYCLERQAGPVDEEHCDPLGRPDDQQRKCSEQPCPARWWAGEWQLCSSSCGPGGLSRRAVLCIRSVGLDEQSALEPPACEHLPRPPTETPCNRHVPCPATWAVGNWSQCSVTCGEGTQRRNVLCTNDTGVPCDEAQQPASEVTCSLPLCRWPLGTLGPEGSGSGSSSHELFNEADFIPHHLAPRPSPASSPKPGTMGNAIEEEAPELDLPGPVFVDDFYYDYNFINFHEDLSYGPSEEPDLDLAGTGDRTPPPHSHPAAPSTGSPVPATEPPAAKEEGVLGPWSPSPWPSQAGRSPPPPSEQTPGNPLINFLPEEDTPIGAPDLGLPSLSWPRVSTDGLQTPATPESQNDFPVGKDSQSQLPPPWRDRTNEVFKDDEEPKGRGAPHLPPRPSSTLPPLSPVGSTHSSPSPDVAELWTGGTVAWEPALEGGLGPVDSELWPTVGVASLLPPPIAPLPEMKVRDSSLEPGTPSFPTPGPGSWDLQTVAVWGTFLPTTLTGLGHMPEPALNPGPKGQPESLSPEVPLSSRLLSTPAWDSPANSHRVPETQPLAPSLAEAGPPADPLVVRNAGWQAGNWSECSTTCGLGAVWRPVRCSSGRDEDCAPAGRPQPARRCHLRPCATWHSGNWSKCSRSCGGGSSVRDVQCVDTRDLRPLRPFHCQPGPAKPPAHRPCGAQPCLSWYTSSWRECSEACGGGEQQRLVTCPEPGLCEEALRPNTTRPCNTHPCTQWVVGPWGQCSGPCGGGVQRRLVKCVNTQTGLPEEDSDQCGHEAWPESSRPCGTEDCEPVEPPRCERDRLSFGFCETLRLLGRCQLPTIRTQCCRSCSPPSHGAPSRGHQRVARR

SEQ ID NO: 2 - protein, 1454 aa, homo sapiens
MHQRSVSKEKWVETLVVADAKMVEYHGQPQVESYVLTIMNMVAGLFHDPSIGNPIHITIVRLVLLEDEEEDLKITHHADNTLKSFCKWQKSINMKGDAHPLHHDTAILLTRKDLCAAMNRPCETLGLSHVAGMCQPHRSCSINEDTGLPLAFTVAHELGHSFGIQHDGSGNDCEPVGKRPFIMSPQLLYDAAPLTWSRCSRQYITRFLDRGWGLCLDDPPAKDIIDFPSVPPGVLYDVSHQCRLQYGAYSAFCEDMDNVCHTLWCSVGTTCHSKLDAAVDGTRCGENKWCLSGECVPVGFRPEAVDGGWSGWSAWSICSRSCGMGVQSAERQCTQPTPKYKGRYCVGERKRFRLCNLQACPAGRPSFRHVQCSHFDAMLYKGQLHTWVPVVNDVNPCELHCRPANEYFAEKLRDAVVDGTPCYQVRASRDLCINGICKNVGCDFEIDSGAMEDRCGVCHGNGSTCHTVSGTFEEAEGLGYVDVGLIPAGAREIRIQEVAEAANFLALRSEDPEKYFLNGGWTIQWNGDYQVAGTTFTYARRGNWENLTSPGPTKEPVWIQLLFQESNPGVHYEYTIHREAGGHDEVPPPVFSWHYGPWTKCTVTCGRGVQRQNVYCLERQAGPVDEEHCDPLGRPDDQQRKCSEQPCPARWWAGEWQLCSSSCGPGGLSRRAVLCIRSVGLDEQSALEPPACEHLPRPPTETPCNRHVPCPATWAVGNWSQCSVTCGEGTQRRNVLCTNDTGVPCDEAQQPASEVTCSLPLCRWPLGTLGPEGSGSGSSSHELFNEADFIPHHLAPRPSPASSPKPGTMGNAIEEEAPELDLPGPVFVDDFYYDYNFINFHEDLSYGPSEEPDLDLAGTGDRTPPPHSHPAAPSTGSPVPATEPPAAKEEGVLGPWSPSPWPSQAGRSPPPPSEQTPGNPLINFLPEEDTPIGAPDLGLPSLSWPRVSTDGLQTPATPESQNDFPVGKDSQSQLPPPWRDRTNEVFKDDEEPKGRGAPHLPPRPSSTLPPLSPVGSTHSSPSPDVAELWTGGTVAWEPALEGGLGPVDSELWPTVGVASLLPPPIAPLPEMKVRDSSLEPGTPSFPTPGPGSWDLQTVAVWGTFLPTTLTGLGHMPEPALNPGPKGQPESLSPEVPLSSRLLSTPAWDSPANSHRVPETQPLAPSLAEAGPPADPLVVRNAGWQAGNWSECSTTCGLGAVWRPVRCSSGRDEDCAPAGRPQPARRCHLRPCATWHSGNWSKCSRSCGGGSSVRDVQCVDTRDLRPLRPFHCQPGPAKPPAHRPCGAQPCLSWYTSSWRECSEACGGGEQQRLVTCPEPGLCEEALRPNTTRPCNTHPCTQWVVGPWGQCSGPCGGGVQRRLVKCVNTQTGLPEEDSDQCGHEAWPESSRPCGTEDCEPVEPPRCERDRLSFGFCETLRLLGRCQLPTIRTQCCRSCSPPSHGAPSRGHQRVARR

SEQ ID NO: 3 - protein, 599 aa, HOMO SAPIENS
MPGGPSPRSPAPLLRPLLLLLCALAPGAPGPAPGRATEGRAALDIVHPVRVDAGGSFLSYELWPRALRKRDVSVRRDAPAFYELQYRGRELRFNLTANQHLLAPGFVSETRRRGGLGRAHIRAHTPACHLLGEVQDPELEGGLAAISACDGLKGVFQLSNEDYFIEPLDSAPARPGHAQPHVVYKRQAPERLAQRGDSSAPSTCGVQVYPELESRRERWEQRQQWRRPRLRRLHQRSVSKEKWVETLVVADAKMVEYHGQPQVESYVLTIMNMVAGLFHDPSIGNPIHITIVRLVLLEDEEEDLKITHHADNTLKSFCKWQKSINMKGDAHPLHHDTAILLTRKDLCAAMNRPCETLGLSHVAGMCQPHRSCSINEDTGLPLAFTVAHELGHSFGIQHDGSGNDCEPVGKRPFIMSPQLLYDAAPLTWSRCSRQYITRFLDRGWGLCLDDPPAKDIIDFPSVPPGVLYDVSHQCRLQYGAYSAFCEDMDNVCHTLWCSVGTTCHSKLDAAVDGTRCGENKWCLSGECVPVGFRPEAVDGGWSGWSAWSICSRSCGMGVQSAERQCTQPTPKYKGRYCVGERKRFRLCNLQACPAGRPSF

SEQ ID NO: 4 - protein, 367 aa, HOMO SAPIENS
MHQRSVSKEKWVETLVVADAKMVEYHGQPQVESYVLTIMNMVAGLFHDPSIGNPIHITIVRLVLLEDEEEDLKITHHADNTLKSFCKWQKSINMKGDAHPLHHDTAILLTRKDLCAAMNRPCETLGLSHVAGMCQPHRSCSINEDTGLPLAFTVAHELGHSFGIQHDGSGNDCEPVGKRPFIMSPQLLYDAAPLTWSRCSRQYITRFLDRGWGLCLDDPPAKDIIDFPSVPPGVLYDVSHQCRLQYGAYSAFCEDMDNVCHTLWCSVGTTCHSKLDAAVDGTRCGENKWCLSGECVPVGFRPEAVDGGWSGWSAWSICSRSCGMGVQSAERQCTQPTPKYKGRYCVGERKRFRLCNLQACPAGRPSF

SEQ ID NO: 5 - protein, 1076 aa, homo sapiens
MQFVSWATLLTLLVRDLAEMGSPDAAAAVRKDRLHPRQVKLLETLGEYEIVSPIRVNALGEPFPTNVHFKRTRRSINSATDPWPAFASSSSSSTSSQAHYRLSAFGQQFLFNLTANAGFIAPLFTVTLLGTPGVNQTKFYSEEEAELKHCFYKGYVNTNSEHTAVISLCSGMLGTFRSHDGDYFIEPLQSMDEQEDEEEQNKPHIIYRRSAPQREPSTGRHACDTSEHKNRHSKDKKKTRARKWGERINLAGDVAALNSGLATEAFSAYGNKTDNTREKRTHRRTKRFLSYPRFVEVLVVADNRMVSYHGENLQHYILTLMSIVASIYKDPSIGNLINIVIVNLIVIHNEQDGPSISFNAQTTLKNLCQWQHSKNSPGGIHHDTAVLLTRQDICRAHDKCDTLGLAELGTICDPYRSCSISEDSGLSTAFTIAHELGHVFNMPHDDNNKCKEEGVKSPQHVMAPTLNFYTNPWMWSKCSRKYITEFLDTGYGECLLNEPESRPYPLPVQLPGILYNVNKQCELIFGPGSQVCPYMMQCRRLWCNNVNGVHKGCRTQHTPWADGTECEPGKHCKYGFCVPKEMDVPVTDGSWGSWSPFGTCSRTCGGGIKTAIRECNRPEPKNGGKYCVGRRMKFKSCNTEPCLKQKRDFRDEQCAHFDGKHFNINGLLPNVRWVPKYSGILMKDRCKLFCRVAGNTAYYQLRDRVIDGTPCGQDTNDICVQGLCRQAGCDHVLNSKARRDKCGVCGGDNSSCKTVAGTFNTVHYGYNTVVRIPAGATNIDVRQHSFSGETDDDNYLALSSSKGEFLLNGNFVVTMAKREIRIGNAVVEYSGSETAVERINSTDRIEQELLLQVLSVGKLYNPDVRYSFNIPIEDKPQQFYWNSHGPWQACSKPCQGERKRKLVCTRESDQLTVSDQRCDRLPQPGHITEPCGTDCDLRWHVASRSECSAQCGLGYRTLDIYCAKYSRLDGKTEKVDDGFCSSHPKPSNREKCSGECNTGGWRYSAWTECSKSCDGGTQRRRAICVNTRNDVLDDSKCTHQEKVTIQRCSEFPCPQWKSGDWSEVRWEGCYFPCYFP

SEQ ID NO: 6 - protein, 785 aa, homo sapiens
MLSYPRFVEVLVVADNRMVSYHGENLQHYILTLMSIVASIYKDPSIGNLINIVIVNLIVIHNEQDGPSISFNAQTTLKNLCQWQHSKNSPGGIHHDTAVLLTRQDICRAHDKCDTLGLAELGTICDPYRSCSISEDSGLSTAFTIAHELGHVFNMPHDDNNKCKEEGVKSPQHVMAPTLNFYTNPWMWSKCSRKYITEFLDTGYGECLLNEPESRPYPLPVQLPGILYNVNKQCELIFGPGSQVCPYMMQCRRLWCNNVNGVHKGCRTQHTPWADGTECEPGKHCKYGFCVPKEMDVPVTDGSWGSWSPFGTCSRTCGGGIKTAIRECNRPEPKNGGKYCVGRRMKFKSCNTEPCLKQKRDFRDEQCAHFDGKHFNINGLLPNVRWVPKYSGILMKDRCKLFCRVAGNTAYYQLRDRVIDGTPCGQDTNDICVQGLCRQAGCDHVLNSKARRDKCGVCGGDNSSCKTVAGTFNTVHYGYNTVVRIPAGATNIDVRQHSFSGETDDDNYLALSSSKGEFLLNGNFVVTMAKREIRIGNAVVEYSGSETAVERINSTDRIEQELLLQVLSVGKLYNPDVRYSFNIPIEDKPQQFYWNSHGPWQACSKPCQGERKRKLVCTRESDQLTVSDQRCDRLPQPGHITEPCGTDCDLRWHVASRSECSAQCGLGYRTLDIYCAKYSRLDGKTEKVDDGFCSSHPKPSNREKCSGECNTGGWRYSAWTECSKSCDGGTQRRRAICVNTRNDVLDDSKCTHQEKVTIQRCSEFPCPQWKSGDWSEVRWEGCYFP

SEQ ID NO: 7 - protein, 649 aa, homo sapiens
MQFVSWATLLTLLVRDLAEMGSPDAAAAVRKDRLHPRQVKLLETLGEYEIVSPIRVNALGEPFPTNVHFKRTRRSINSATDPWPAFASSSSSSTSSQAHYRLSAFGQQFLFNLTANAGFIAPLFTVTLLGTPGVNQTKFYSEEEAELKHCFYKGYVNTNSEHTAVISLCSGMLGTFRSHDGDYFIEPLQSMDEQEDEEEQNKPHIIYRRSAPQREPSTGRHACDTSEHKNRHSKDKKKTRARKWGERINLAGDVAALNSGLATEAFSAYGNKTDNTREKRTHRRTKRFLSYPRFVEVLVVADNRMVSYHGENLQHYILTLMSIVASIYKDPSIGNLINIVIVNLIVIHNEQDGPSISFNAQTTLKNLCQWQHSKNSPGGIHHDTAVLLTRQDICRAHDKCDTLGLAELGTICDPYRSCSISEDSGLSTAFTIAHELGHVFNMPHDDNNKCKEEGVKSPQHVMAPTLNFYTNPWMWSKCSRKYITEFLDTGYGECLLNEPESRPYPLPVQLPGILYNVNKQCELIFGPGSQVCPYMMQCRRLWCNNVNGVHKGCRTQHTPWADGTECEPGKHCKYGFCVPKEMDVPVTDGSWGSWSPFGTCSRTCGGGIKTAIRECNRPEPKNGGKYCVGRRMKFKSCNTEPCLKQKRDF

SEQ ID NO: 8 - protein, 362 aa, homo sapiens
MLSYPRFVEVLVVADNRMVSYHGENLQHYILTLMSIVASIYKDPSIGNLINIVIVNLIVIHNEQDGPSISFNAQTTLKNLCQWQHSKNSPGGIHHDTAVLLTRQDICRAHDKCDTLGLAELGTICDPYRSCSISEDSGLSTAFTIAHELGHVFNMPHDDNNKCKEEGVKSPQHVMAPTLNFYTNPWMWSKCSRKYITEFLDTGYGECLLNEPESRPYPLPVQLPGILYNVNKQCELIFGPGSQVCPYMMQCRRLWCNNVNGVHKGCRTQHTPWADGTECEPGKHCKYGFCVPKEMDVPVTDGSWGSWSPFGTCSRTCGGGIKTAIRECNRPEPKNGGKYCVGRRMKFKSCNTEPCLKQKRDF

SEQ ID NO: 9 - protein, 1103 aa, homo sapiens
MAPACQILRWALALGLGLMFEVTHAFRSQDEFLSSLESYEIAFPTRVDHNGALLAFSPPPPRRQRRGTGATAESRLFYKVASPSTHFLLNLTRSSRLLAGHVSVEYWTREGLAWQRAARPHCLYAGHLQGQASTSHVAISTCGGLHGLIVADEEEYLIEPLHGGPKGSRSPEESGPHVVYKRSSLRHPHLDTACGVRDEKPWKGRPWWLRTLKPPPARPLGNETERGQPGLKRSVSRERYVETLVVADKMMVAYHGRRDVEQYVLAIMNIVAKLFQDSSLGSTVNILVTRLILLTEDQPTLEITHHAGKSLDSFCKWQKSIVNHSGHGNAIPENGVANHDTAVLITRYDICIYKNKPCGTLGLAPVGGMCERERSCSVNEDIGLATAFTIAHEIGHTFGMNHDGVGNSCGARGQDPAKLMAAHITMKTNPFVWSSCSRDYITSFLDSGLGLCLNNRPPRQDFVYPTVAPGQAYDADEQCRFQHGVKSRQCKYGEVCSELWCLSKSNRCITNSIPAAEGTLCQTHTIDKGWCYKRVCVPFGSRPEGVDGAWGPWTPWGDCSRTCGGGVSSSSRHCDSPRPTIGGKYCLGERRRHRSCNTDDCPPGSQDFREVQCSEFDSIPFRGKFYKWKTYRGGGVKACSLTCLAEGFNFYTERAAAVVDGTPCRPDTVDICVSGECKHVGCDRVLGSDLREDKCRVCGGDGSACETIEGVFSPASPGAGYEDVVWIPKGSVHIFIQDLNLSLSHLALKGDQESLLLEGLPGTPQPHRLPLAGTTFQLRQGPDQVQSLEALGPINASLIVMVLARTELPALRYRFNAPIARDSLPPYSWHYAPWTKCSAQCAGGSQVQAVECRNQLDSSAVAPHYCSAHSKLPKRQRACNTEPCPPDWVVGNWSLCSRSCDAGVRSRSVVCQRRVSAAEEKALDDSACPQPRPPVLEACHGPTCPPEWAALDWSECTPSCGPGLRHRVVLCKSADHRATLPPAHCSPAAKPPATMRCNLRRCPPARWVAGEWGECSAQCGVGQRQRSVRCTSHTGQASHECTEALRPPTTQQCEAKCDSPTPGDGPEECKDVNKVAYCPLVLKFQFCSRAYFRQMCCKTCQGH

SEQ ID NO: 10 - protein, 871 aa, homo sapiens
MSVSRERYVETLVVADKMMVAYHGRRDVEQYVLAIMNIVAKLFQDSSLGSTVNILVTRLILLTEDQPTLEITHHAGKSLDSFCKWQKSIVNHSGHGNAIPENGVANHDTAVLITRYDICIYKNKPCGTLGLAPVGGMCERERSCSVNEDIGLATAFTIAHEIGHTFGMNHDGVGNSCGARGQDPAKLMAAHITMKTNPFVWSSCSRDYITSFLDSGLGLCLNNRPPRQDFVYPTVAPGQAYDADEQCRFQHGVKSRQCKYGEVCSELWCLSKSNRCITNSIPAAEGTLCQTHTIDKGWCYKRVCVPFGSRPEGVDGAWGPWTPWGDCSRTCGGGVSSSSRHCDSPRPTIGGKYCLGERRRHRSCNTDDCPPGSQDFREVQCSEFDSIPFRGKFYKWKTYRGGGVKACSLTCLAEGFNFYTERAAAVVDGTPCRPDTVDICVSGECKHVGCDRVLGSDLREDKCRVCGGDGSACETIEGVFSPASPGAGYEDVVWIPKGSVHIFIQDLNLSLSHLALKGDQESLLLEGLPGTPQPHRLPLAGTTFQLRQGPDQVQSLEALGPINASLIVMVLARTELPALRYRFNAPIARDSLPPYSWHYAPWTKCSAQCAGGSQVQAVECRNQLDSSAVAPHYCSAHSKLPKRQRACNTEPCPPDWVVGNWSLCSRSCDAGVRSRSVVCQRRVSAAEEKALDDSACPQPRPPVLEACHGPTCPPEWAALDWSECTPSCGPGLRHRVVLCKSADHRATLPPAHCSPAAKPPATMRCNLRRCPPARWVAGEWGECSAQCGVGQRQRSVRCTSHTGQASHECTEALRPPTTQQCEAKCDSPTPGDGPEECKDVNKVAYCPLVLKFQFCSRAYFRQMCCKTCQGH

SEQ ID NO: 11 - protein, 608 aa, homo sapiens
MAPACQILRWALALGLGLMFEVTHAFRSQDEFLSSLESYEIAFPTRVDHNGALLAFSPPPPRRQRRGTGATAESRLFYKVASPSTHFLLNLTRSSRLLAGHVSVEYWTREGLAWQRAARPHCLYAGHLQGQASTSHVAISTCGGLHGLIVADEEEYLIEPLHGGPKGSRSPEESGPHVVYKRSSLRHPHLDTACGVRDEKPWKGRPWWLRTLKPPPARPLGNETERGQPGLKRSVSRERYVETLVVADKMMVAYHGRRDVEQYVLAIMNIVAKLFQDSSLGSTVNILVTRLILLTEDQPTLEITHHAGKSLDSFCKWQKSIVNHSGHGNAIPENGVANHDTAVLITRYDICIYKNKPCGTLGLAPVGGMCERERSCSVNEDIGLATAFTIAHEIGHTFGMNHDGVGNSCGARGQDPAKLMAAHITMKTNPFVWSSCSRDYITSFLDSGLGLCLNNRPPRQDFVYPTVAPGQAYDADEQCRFQHGVKSRQCKYGEVCSELWCLSKSNRCITNSIPAAEGTLCQTHTIDKGWCYKRVCVPFGSRPEGVDGAWGPWTPWGDCSRTCGGGVSSSSRHCDSPRPTIGGKYCLGERRRHRSCNTDDCPPGSQDF

SEQ ID NO: 12 - protein, 376 aa, homo sapiens
MSVSRERYVETLVVADKMMVAYHGRRDVEQYVLAIMNIVAKLFQDSSLGSTVNILVTRLILLTEDQPTLEITHHAGKSLDSFCKWQKSIVNHSGHGNAIPENGVANHDTAVLITRYDICIYKNKPCGTLGLAPVGGMCERERSCSVNEDIGLATAFTIAHEIGHTFGMNHDGVGNSCGARGQDPAKLMAAHITMKTNPFVWSSCSRDYITSFLDSGLGLCLNNRPPRQDFVYPTVAPGQAYDADEQCRFQHGVKSRQCKYGEVCSELWCLSKSNRCITNSIPAAEGTLCQTHTIDKGWCYKRVCVPFGSRPEGVDGAWGPWTPWGDCSRTCGGGVSSSSRHCDSPRPTIGGKYCLGERRRHRSCNTDDCPPGSQDF

SEQ ID NO: 13 - protein, 1072 aa, homo sapiens
MKPRARGWRGLAALWMLLAQVAEQAPACAMGPAAAAPGSPSVPRPPPPAERPGWMEKGEYDLVSAYEVDHRGDYVSHEIMHHQRRRRAVAVSEVESLHLRLKGPRHDFHMDLRTSSSLVAPGFIVQTLGKTGTKSVQTLPPEDFCFYQGSLRSHRNSSVALSTCQGLSGMIRTEEADYFLRPLPSHLSWKLGRAAQGSSPSHVLYKRSTEPHAPGASEVLVTSRTWELAHQPLHSSDLRLGLPQKQHFCGRRKKYMPQPPKEDLFILPDEYKSCLRHKRSLLRSHRNEELNVETLVVVDKKMMQNHGHENITTYVLTILNMVSALFKDGTIGGNINIAIVGLILLEDEQPGLVISHHADHTLSSFCQWQSGLMGKDGTRHDHAILLTGLDICSWKNEPCDTLGFAPISGMCSKYRSCTINEDTGLGLAFTIAHESGHNFGMIHDGEGNMCKKSEGNIMSPTLAGRNGVFSWSPCSRQYLHKFLSTAQAICLADQPKPVKEYKYPEKLPGELYDANTQCKWQFGEKAKLCMLDFKKDICKALWCHRIGRKCETKFMPAAEGTICGHDMWCRGGQCVKYGDEGPKPTHGHWSDWSSWSPCSRTCGGGVSHRSRLCTNPKPSHGGKFCEGSTRTLKLCNSQKCPRDSVDFRAAQCAEHNSRRFRGRHYKWKPYTQVEDQDLCKLYCIAEGFDFFFSLSNKVKDGTPCSEDSRNVCIDGICERVGCDNVLGSDAVEDVCGVCNGNNSACTIHRGLYTKHHHTNQYYHMVTIPSGARSIRIYEMNVSTSYISVRNALRRYYLNGHWTVDWPGRYKFSGTTFDYRRSYNEPENLIATGPTNETLIVELLFQGRNPGVAWEYSMPRLGTEKQPPAQPSYTWAIVRSECSVSCGGGQMTVREGCYRDLKFQVNMSFCNPKTRPVTGLVPCKVSACPPSWSVGNWSACSRTCGGGAQSRPVQCTRRVHYDSEPVPASLCPQPAPSSRQACNSQSCPPAWSAGPWAECSHTCGKGWRKRAVACKSTNPSARAQLLPDAVCTSEPKPRMHEACLLQRCHKPKKLQWLVSAWSQVGALVSRERG

SEQ ID NO: 14 - protein, 795 aa, homo sapiens
MRSLLRSHRNEELNVETLVVVDKKMMQNHGHENITTYVLTILNMVSALFKDGTIGGNINIAIVGLILLEDEQPGLVISHHADHTLSSFCQWQSGLMGKDGTRHDHAILLTGLDICSWKNEPCDTLGFAPISGMCSKYRSCTINEDTGLGLAFTIAHESGHNFGMIHDGEGNMCKKSEGNIMSPTLAGRNGVFSWSPCSRQYLHKFLSTAQAICLADQPKPVKEYKYPEKLPGELYDANTQCKWQFGEKAKLCMLDFKKDICKALWCHRIGRKCETKFMPAAEGTICGHDMWCRGGQCVKYGDEGPKPTHGHWSDWSSWSPCSRTCGGGVSHRSRLCTNPKPSHGGKFCEGSTRTLKLCNSQKCPRDSVDFRAAQCAEHNSRRFRGRHYKWKPYTQVEDQDLCKLYCIAEGFDFFFSLSNKVKDGTPCSEDSRNVCIDGICERVGCDNVLGSDAVEDVCGVCNGNNSACTIHRGLYTKHHHTNQYYHMVTIPSGARSIRIYEMNVSTSYISVRNALRRYYLNGHWTVDWPGRYKFSGTTFDYRRSYNEPENLIATGPTNETLIVELLFQGRNPGVAWEYSMPRLGTEKQPPAQPSYTWAIVRSECSVSCGGGQMTVREGCYRDLKFQVNMSFCNPKTRPVTGLVPCKVSACPPSWSVGNWSACSRTCGGGAQSRPVQCTRRVHYDSEPVPASLCPQPAPSSRQACNSQSCPPAWSAGPWAECSHTCGKGWRKRAVACKSTNPSARAQLLPDAVCTSEPKPRMHEACLLQRCHKPKKLQWLVSAWSQVGALVSRERG

SEQ ID NO: 15 - protein, 647 aa, homo sapiens
MKPRARGWRGLAALWMLLAQVAEQAPACAMGPAAAAPGSPSVPRPPPPAERPGWMEKGEYDLVSAYEVDHRGDYVSHEIMHHQRRRRAVAVSEVESLHLRLKGPRHDFHMDLRTSSSLVAPGFIVQTLGKTGTKSVQTLPPEDFCFYQGSLRSHRNSSVALSTCQGLSGMIRTEEADYFLRPLPSHLSWKLGRAAQGSSPSHVLYKRSTEPHAPGASEVLVTSRTWELAHQPLHSSDLRLGLPQKQHFCGRRKKYMPQPPKEDLFILPDEYKSCLRHKRSLLRSHRNEELNVETLVVVDKKMMQNHGHENITTYVLTILNMVSALFKDGTIGGNINIAIVGLILLEDEQPGLVISHHADHTLSSFCQWQSGLMGKDGTRHDHAILLTGLDICSWKNEPCDTLGFAPISGMCSKYRSCTINEDTGLGLAFTIAHESGHNFGMIHDGEGNMCKKSEGNIMSPTLAGRNGVFSWSPCSRQYLHKFLSTAQAICLADQPKPVKEYKYPEKLPGELYDANTQCKWQFGEKAKLCMLDFKKDICKALWCHRIGRKCETKFMPAAEGTICGHDMWCRGGQCVKYGDEGPKPTHGHWSDWSSWSPCSRTCGGGVSHRSRLCTNPKPSHGGKFCEGSTRTLKLCNSQKCPRDSVDF

SEQ ID NO: 16 - protein, 370 aa, homo sapiens
MRSLLRSHRNEELNVETLVVVDKKMMQNHGHENITTYVLTILNMVSALFKDGTIGGNINIAIVGLILLEDEQPGLVISHHADHTLSSFCQWQSGLMGKDGTRHDHAILLTGLDICSWKNEPCDTLGFAPISGMCSKYRSCTINEDTGLGLAFTIAHESGHNFGMIHDGEGNMCKKSEGNIMSPTLAGRNGVFSWSPCSRQYLHKFLSTAQAICLADQPKPVKEYKYPEKLPGELYDANTQCKWQFGEKAKLCMLDFKKDICKALWCHRIGRKCETKFMPAAEGTICGHDMWCRGGQCVKYGDEGPKPTHGHWSDWSSWSPCSRTCGGGVSHRSRLCTNPKPSHGGKFCEGSTRTLKLCNSQKCPRDSVDF

SEQ ID NO: 17 - protein, 1221 aa, homo sapiens
MECALLLACAFPAAGSGPPRGLAGLGRVAKALQLCCLCCASVAAALASDSSSGASGLNDDYVFVTPVEVDSAGSYISHDILHNGRKKRSAQNARSSLHYRFSAFGQELHLELKPSAILSSHFIVQVLGKDGASETQKPEVQQCFYQGFIRNDSSSSVAVSTCAGLSGLIRTRKNEFLISPLPQLLAQEHNYSSPAGHHPHVLYKRTAEEKIQRYRGYPGSGRNYPGYSPSHIPHASQSRETEYHHRRLQKQHFCGRRKKYAPKPPTEDTYLRFDEYGSSGRPRRSAGKSQKGLNVETLVVADKKMVEKHGKGNVTTYILTVMNMVSGLFKDGTIGSDINVVVVSLILLEQEPGGLLINHHADQSLNSFCQWQSALIGKNGKRHDHAILLTGFDICSWKNEPCDTLGFAPISGMCSKYRSCTINEDTGLGLAFTIAHESGHNFGMIHDGEGNPCRKAEGNIMSPTLTGNNGVFSWSSCSRQYLKKFLSTPQAGCLVDEPKQAGQYKYPDKLPGQIYDADTQCKWQFGAKAKLCSLGFVKDICKSLWCHRVGHRCETKFMPAAEGTVCGLSMWCRQGQCVKFGELGPRPIHGQWSAWSKWSECSRTCGGGVKFQERHCNNPKPQYGGLFCPGSSRIYQLCNINPCNENSLDFRAQQCAEYNSKPFRGWFYQWKPYTKVEEEDRCKLYCKAENFEFFFAMSGKVKDGTPCSPNKNDVCIDGVCELVGCDHELGSKAVSDACGVCKGDNSTCKFYKGLYLNQHKANEYYPVVLIPAGARSIEIQELQVSSSYLAVRSLSQKYYLTGGWSIDWPGEFPFAGTTFEYQRSFNRPERLYAPGPTNETLVFEILMQGKNPGIAWKYALPKVMNGTPPATKRPAYTWSIVQSECSVSCGGGYINVKAICLRDQNTQVNSSFCSAKTKPVTEPKICNAFSCPAYWMPGEWSTCSKACAGGQQSRKIQCVQKKPFQKEEAVLHSLCPVSTPTQVQACNSHACPPQWSLGPWSQCSKTCGRGVRKRELLCKGSAAETLPESQCTSLPRPELQEGCVLGRCPKNSRLQWVASSWSECSATCGLGVRKREMKCSEKGFQGKLITFPERRCRNIKKPNLDLEETCNRRACPAHPVYNMVAGWYSLPWQQCTVTCGGGVQTRSVHCVQQGRPSSSCLLHQKPPVLRACNTNFCPAPEKREDPSCVDFFNWCHLVPQHGVCNHKFYGKQCCKSCTRKI

SEQ ID NO: 18 - protein, 938 aa, homo sapiens
MSAGKSQKGLNVETLVVADKKMVEKHGKGNVTTYILTVMNMVSGLFKDGTIGSDINVVVVSLILLEQEPGGLLINHHADQSLNSFCQWQSALIGKNGKRHDHAILLTGFDICSWKNEPCDTLGFAPISGMCSKYRSCTINEDTGLGLAFTIAHESGHNFGMIHDGEGNPCRKAEGNIMSPTLTGNNGVFSWSSCSRQYLKKFLSTPQAGCLVDEPKQAGQYKYPDKLPGQIYDADTQCKWQFGAKAKLCSLGFVKDICKSLWCHRVGHRCETKFMPAAEGTVCGLSMWCRQGQCVKFGELGPRPIHGQWSAWSKWSECSRTCGGGVKFQERHCNNPKPQYGGLFCPGSSRIYQLCNINPCNENSLDFRAQQCAEYNSKPFRGWFYQWKPYTKVEEEDRCKLYCKAENFEFFFAMSGKVKDGTPCSPNKNDVCIDGVCELVGCDHELGSKAVSDACGVCKGDNSTCKFYKGLYLNQHKANEYYPVVLIPAGARSIEIQELQVSSSYLAVRSLSQKYYLTGGWSIDWPGEFPFAGTTFEYQRSFNRPERLYAPGPTNETLVFEILMQGKNPGIAWKYALPKVMNGTPPATKRPAYTWSIVQSECSVSCGGGYINVKAICLRDQNTQVNSSFCSAKTKPVTEPKICNAFSCPAYWMPGEWSTCSKACAGGQQSRKIQCVQKKPFQKEEAVLHSLCPVSTPTQVQACNSHACPPQWSLGPWSQCSKTCGRGVRKRELLCKGSAAETLPESQCTSLPRPELQEGCVLGRCPKNSRLQWVASSWSECSATCGLGVRKREMKCSEKGFQGKLITFPERRCRNIKKPNLDLEETCNRRACPAHPVYNMVAGWYSLPWQQCTVTCGGGVQTRSVHCVQQGRPSSSCLLHQKPPVLRACNTNFCPAPEKREDPSCVDFFNWCHLVPQHGVCNHKFYGKQCCKSCTRKI

SEQ ID NO: 19 - protein, 650 aa, homo sapiens
MECALLLACAFPAAGSGPPRGLAGLGRVAKALQLCCLCCASVAAALASDSSSGASGLNDDYVFVTPVEVDSAGSYISHDILHNGRKKRSAQNARSSLHYRFSAFGQELHLELKPSAILSSHFIVQVLGKDGASETQKPEVQQCFYQGFIRNDSSSSVAVSTCAGLSGLIRTRKNEFLISPLPQLLAQEHNYSSPAGHHPHVLYKRTAEEKIQRYRGYPGSGRNYPGYSPSHIPHASQSRETEYHHRRLQKQHFCGRRKKYAPKPPTEDTYLRFDEYGSSGRPRRSAGKSQKGLNVETLVVADKKMVEKHGKGNVTTYILTVMNMVSGLFKDGTIGSDINVVVVSLILLEQEPGGLLINHHADQSLNSFCQWQSALIGKNGKRHDHAILLTGFDICSWKNEPCDTLGFAPISGMCSKYRSCTINEDTGLGLAFTIAHESGHNFGMIHDGEGNPCRKAEGNIMSPTLTGNNGVFSWSSCSRQYLKKFLSTPQAGCLVDEPKQAGQYKYPDKLPGQIYDADTQCKWQFGAKAKLCSLGFVKDICKSLWCHRVGHRCETKFMPAAEGTVCGLSMWCRQGQCVKFGELGPRPIHGQWSAWSKWSECSRTCGGGVKFQERHCNNPKPQYGGLFCPGSSRIYQLCNINPCNENSLDF

SEQ ID NO: 20 - protein, 367 aa, homo sapiens
MSAGKSQKGLNVETLVVADKKMVEKHGKGNVTTYILTVMNMVSGLFKDGTIGSDINVVVVSLILLEQEPGGLLINHHADQSLNSFCQWQSALIGKNGKRHDHAILLTGFDICSWKNEPCDTLGFAPISGMCSKYRSCTINEDTGLGLAFTIAHESGHNFGMIHDGEGNPCRKAEGNIMSPTLTGNNGVFSWSSCSRQYLKKFLSTPQAGCLVDEPKQAGQYKYPDKLPGQIYDADTQCKWQFGAKAKLCSLGFVKDICKSLWCHRVGHRCETKFMPAAEGTVCGLSMWCRQGQCVKFGELGPRPIHGQWSAWSKWSECSRTCGGGVKFQERHCNNPKPQYGGLFCPGSSRIYQLCNINPCNENSLDF

SEQ ID NO: 21 - DNA, 4360 bp, homo sapiens
acctgtggagtgcaagtgtacccagagctggagtctcgacgggagcgttgggagcagcggcagcagtggcggcggccacggctgaggcgtctacaccagcggtcggtcagcaaagagaagtgggtggagaccctggtagtagctgatgccaaaatggtggagtaccacggacagccgcaggttgagagctatgtgctgaccatcatgaacatggtggctggcctgtttcatgaccccagcattgggaaccccatccacatcaccattgtgcgcctggtcctgctggaagatgaggaggaggacctaaagatcacgcaccatgcagacaacaccctgaagagcttctgcaagtggcagaaaagcatcaacatgaagggggatgcccatcccctgcaccatgacactgccatcctgctcaccagaaaggacctgtgtgcagccatgaaccggccctgtgagaccctgggactgtcccatgtggcgggcatgtgccagccgcaccgcagctgcagcatcaacgaggacacgggcctgccgctggccttcactgtagcccacgagctcgggcacagttttggcattcagcatgacggaagcggcaatgactgtgagcccgttgggaaacgacctttcatcatgtctccacagctcctgtacgacgccgctcccctcacctggtcccgctgcagccgccagtatatcaccaggttccttgaccgtgggtggggcctgtgcctggacgaccctcctgccaaggacattatcgacttcccctcggtgccacctggcgtcctctatgatgtaagccaccagtgccgcctccagtacggggcctactctgccttctgcgaggacatggataatgtctgccacacactctggtgctctgtggggaccacctgtcactccaagctggatgcagctgtggacggcacccggtgtggggagaataagtggtgtctcagtggggagtgcgtacccgtgggcttccggcccgaggccgtggatggtggctggtctggctggagcgcctggtccatctgctcacggagctgtggcatgggcgtacagagcgccgagcggcagtgcacgcagcctacgcccaaatacaaaggcagatactgtgtgggtgagcgcaagcgcttccgcctctgcaacctgcaggcctgccctgctggccgcccctccttccgccacgtccagtgcagccactttgacgctatgctctacaagggccagctgcacacatgggtgcccgtggtcaatgacgtgaacccctgcgagctgcactgccggcccgcgaatgagtactttgccgagaagctgcgggacgccgtggtcgatggcaccccctgctaccaggtccgagccagccgggacctctgcatcaacggcatctgtaagaacgtgggctgtgacttcgagattgactccggtgctatggaggaccgctgtggtgtgtgccacggcaacggctccacctgccacaccgtgagcgggaccttcgaggaggccgagggcctggggtatgtggatgtggggctgatcccagcgggcgcacgcgagatccgcatccaagaggttgccgaggctgccaacttcctggcactgcggagcgaggacccggagaagtacttcctcaatggtggctggaccatccagtggaacggggactaccaggtggcagggaccaccttcacatacgcacgcaggggcaactgggagaacctcacgtccccgggtcccaccaaggagcctgtctggatccagctgctgttccaggagagcaaccctggggtgcactacgagtacaccatccacagggaggcaggtggccacgacgaggtcccgccgcccgtgttctcctggcattatgggccctggaccaagtgcacagtcacctgcggcagaggtgtgcagagacagaatgtgtactgcttggagcggcaggcagggcccgtggacgaggagcactgtgaccccctgggccggcctgatgaccaacagaggaagtgcagcgagcagccctgccctgccaggtggtgggcaggtgagtggcagctgtgctccagctcctgcgggcctgggggcctctcccgccgggccgtgctctgcatccgcagcgtggggctggatgagcagagcgccctggagccacccgcctgtgaacaccttccccggccccctactgaaaccccttgcaaccgccatgtaccctgtccggccacctgggctgtggggaactggtctcagtgctcagtgacatgtggggagggcactcagcgccgaaatgtcctctgcaccaatgacaccggtgtcccctgtgacgaggcccagcagccagccagcgaagtcacctgctctctgccactctgtcggtggcccctgggcacactgggccctgaaggctcaggcagcggctcctccagccacgagctcttcaacgaggctgacttcatcccgcaccacctggccccacgcccttcacccgcctcatcacccaagccaggcaccatgggcaacgccattgaggaggaggctccagagctggacctgccggggcccgtgtttgtggacgacttctactacgactacaatttcatcaatttccacgaggatctgtcctacgggccctctgaggagcccgatctagacctggcggggacaggggaccggacacccccaccacacagccatcctgctgcgccctccacgggtagccctgtgcctgccacagagcctcctgcagccaaggaggagggggtactgggaccttggtccccgagcccttggcctagccaggccggccgctccccacccccaccctcagagcagacccctgggaaccctttgatcaatttcctgcctgaggaagacacccccataggggccccagatcttgggctccccagcctgtcctggcccagggtttccactgatggcctgcagacacctgccacccctgagagccaaaatgatttcccagttggcaaggacagccagagccagctgccccctccatggcgggacaggaccaatgaggttttcaaggatgatgaggaacccaagggccgcggagcaccccacctgcccccgagacccagctccacgctgccccctttgtcccctgttggcagcacccactcctctcctagtcctgacgtggcggagctgtggacaggaggcacagtggcctgggagccagctctggagggtggcctggggcctgtggacagtgaactgtggcccactgttggggtggcttctctccttcctcctcccatagcccctctgccagagatgaaggtcagggacagttccctggagccggggactccctccttcccaaccccaggaccaggctcatgggacctgcagactgtggcagtgtgggggaccttcctccccacaaccctgactggcctcgggcacatgcctgagcctgccctgaacccaggacccaagggtcagcctgagtccctcagccctgaggtgcccctgagctctaggctgctgtccacaccagcttgggacagccccgccaacagccacagagtccctgagacccagccgctggctcccagcctggctgaagcggggccccccgcggacccgttggttgtcaggaacgccggctggcaagcgggaaactggagcgagtgctctaccacctgtggcctgggtgcggtctggaggccggtgcgctgtagctccggccgggatgaggactgcgcccccgctggccggccccagcctgcccgccgctgccacctgcggccctgtgccacctggcactcaggcaactggagtaagtgctcccgcagctgcggcggaggttcctcagtgcgggacgtgcagtgtgtggacacacgggacctccggccactgcggcccttccattgtcagcccgggcctgccaagccgcctgcgcaccggccctgcggggcccagccctgcctcagctggtacacatcttcctggagggagtgctccgaggcctgtggcggtggtgagcagcagcgtctagtgacctgcccggagccaggcctctgcgaggaggcgctgagacccaacaccacccggccctgcaacacccacccctgcacgcagtgggtggtggggccctggggccagtgctcaggcccctgtggtggtggtgtccagcggcgcctggtcaagtgtgtcaacacccagacagggctgcccgaggaagacagtgaccagtgtggccacgaggcctggcctgagagctcccggccgtgtggcaccgaggattgtgagcccgtcgagcctccccgctgtgagcgggaccgcctgtccttcgggttctgcgagacgctgcgcctactgggccgctgcc

SEQ ID NO: 22 - DNA, 1798 bp, homo sapiens
atgtcctgagccggacctgggaccaggcagcgaccggacgggacggacgagagcgtgcgcgcggggtcacccggcggccgcccggttcctgccatgcccggcggccccagtccccgcagccccgcgcctttgctgcgccccctcctcctgctcctctgcgctctggctcccggcgcccccggacccgcaccaggacgtgcaaccgagggccgggcggcactggacatcgtgcacccggttcgagtcgacgcggggggctccttcctgtcctacgagctgtggccccgcgcactgcgcaagcgggatgtatctgtgcgccgagacgcgcccgccttctacgagctacaataccgcgggcgcgagctgcgcttcaacctgaccgccaatcagcacctgctggcgcccggctttgtgagcgagacgcggcggcgcggcggcctgggccgcgcgcacatccgggcccacaccccggcctgccacctgcttggcgaggtgcaggaccctgagctcgagggtggcctggcggccatcagcgcctgcgacggcctgaaaggtgtgttccagctctccaacgaggactacttcattgagcccctggacagtgccccggcccggcctggccacgcccagccccatgtggtgtacaagcgtcaggccccggagaggctggcacagcggggtgattccagtgctccaagcacctgtggagtgcaagtgtacccagagctggagtctcgacgggagcgttgggagcagcggcagcagtggcggcggccacggctgaggcgtctacaccagcggtcggtcagcaaagagaagtgggtggagaccctggtagtagctgatgccaaaatggtggagtaccacggacagccgcaggttgagagctatgtgctgaccatcatgaacatggtggctggcctgtttcatgaccccagcattgggaaccccatccacatcaccattgtgcgcctggtcctgctggaagatgaggaggaggacctaaagatcacgcaccatgcagacaacaccctgaagagcttctgcaagtggcagaaaagcatcaacatgaagggggatgcccatcccctgcaccatgacactgccatcctgctcaccagaaaggacctgtgtgcagccatgaaccggccctgtgagaccctgggactgtcccatgtggcgggcatgtgccagccgcaccgcagctgcagcatcaacgaggacacgggcctgccgctggccttcactgtagcccacgagctcgggcacagttttggcattcagcatgacggaagcggcaatgactgtgagcccgttgggaaacgacctttcatcatgtctccacagctcctgtacgacgccgctcccctcacctggtcccgctgcagccgccagtatatcaccaggttccttgaccgtgggtggggcctgtgcctggacgaccctcctgccaaggacattatcgacttcccctcggtgccacctggcgtcctctatgatgtaagccaccagtgccgcctccagtacggggcctactctgccttctgcgaggacatggataatgtctgccacacactctggtgctctgtggggaccacctgtcactccaagctggatgcagctgtggacggcacccggtgtggggagaataagtggtgtctcagtggggagtgcgtacccgtgggcttccggcccgaggccgtggatggtggctggtctggctggagcgcctggtccatctgctcacggagctgtggcatgggcgtacagagcgccgagcggcagtgcacgcagccta

SEQ ID NO: 23 - DNA, 1099 bp, homo sapiens
acctgtggagtgcaagtgtacccagagctggagtctcgacgggagcgttgggagcagcggcagcagtggcggcggccacggctgaggcgtctacaccagcggtcggtcagcaaagagaagtgggtggagaccctggtagtagctgatgccaaaatggtggagtaccacggacagccgcaggttgagagctatgtgctgaccatcatgaacatggtggctggcctgtttcatgaccccagcattgggaaccccatccacatcaccattgtgcgcctggtcctgctggaagatgaggaggaggacctaaagatcacgcaccatgcagacaacaccctgaagagcttctgcaagtggcagaaaagcatcaacatgaagggggatgcccatcccctgcaccatgacactgccatcctgctcaccagaaaggacctgtgtgcagccatgaaccggccctgtgagaccctgggactgtcccatgtggcgggcatgtgccagccgcaccgcagctgcagcatcaacgaggacacgggcctgccgctggccttcactgtagcccacgagctcgggcacagttttggcattcagcatgacggaagcggcaatgactgtgagcccgttgggaaacgacctttcatcatgtctccacagctcctgtacgacgccgctcccctcacctggtcccgctgcagccgccagtatatcaccaggttccttgaccgtgggtggggcctgtgcctggacgaccctcctgccaaggacattatcgacttcccctcggtgccacctggcgtcctctatgatgtaagccaccagtgccgcctccagtacggggcctactctgccttctgcgaggacatggataatgtctgccacacactctggtgctctgtggggaccacctgtcactccaagctggatgcagctgtggacggcacccggtgtggggagaataagtggtgtctcagtggggagtgcgtacccgtgggcttccggcccgaggccgtggatggtggctggtctggctggagcgcctggtccatctgctcacggagctgtggcatgggcgtacagagcgccgagcggcagtgcacgcagccta

SEQ ID NO: 24 - DNA, 2353 bp, homo sapiens
ttatcctatccacggtttgtagaagtcttggtggtggcagacaacagaatggtttcataccatggagaaaaccttcaacactatattttaactttaatgtcaattgtagcctctatctataaagacccaagtattggaaatttaattaatattgttattgtgaacttaattgtgattcataatgaacaggatgggccttccatatcttttaatgctcagacaacattaaaaaacttttgccagtggcagcattcgaagaacagtccaggtggaatccatcatgatactgctgttctcttaacaagacaggatatctgcagagctcacgacaaatgtgataccttaggcctggctgaactgggaaccatttgtgatccctatagaagctgttctattagtgaagatagtggattgagtacagcttttacgatcgcccatgagctgggccatgtgtttaacatgcctcatgatgacaacaacaaatgtaaagaagaaggagttaagagtccccagcatgtcatggctccaacactgaacttctacaccaacccctggatgtggtcaaagtgtagtcgaaaatatatcactgagtttttagacactggttatggcgagtgtttgcttaacgaacctgaatccagaccctaccctttgcctgtccaactgccaggcatcctttacaacgtgaataaacaatgtgaattgatttttggaccaggttctcaggtgtgcccatatatgatgcagtgcagacggctctggtgcaataacgtcaatggagtacacaaaggctgccggactcagcacacaccctgggccgatgggacggagtgcgagcctggaaagcactgcaagtatggattttgtgttcccaaagaaatggatgtccccgtgacagatggatcctggggaagttggagtccctttggaacctgctccagaacatgtggagggggcatcaaaacagccattcgagagtgcaacagaccagaaccaaaaaatggtggaaaatactgtgtaggacgtagaatgaaatttaagtcctgcaacacggagccatgtctcaagcagaagcgagacttccgagatgaacagtgtgctcactttgacgggaagcattttaacatcaacggtctgcttcccaatgtgcgctgggtccctaaatacagtggaattctgatgaaggaccggtgcaagttgttctgcagagtggcagggaacacagcctactatcagcttcgagacagagtgatagatggaactccttgtggccaggacacaaatgatatctgtgtccagggcctttgccggcaagctggatgcgatcatgttttaaactcaaaagcccggagagataaatgtggggtttgtggtggcgataattcttcatgcaaaacagtggcaggaacatttaatacagtacattatggttacaatactgtggtccgaattccagctggtgctaccaatattgatgtgcggcagcacagtttctcaggggaaacagacgatgacaactacttagctttatcaagcagtaaaggtgaattcttgctaaatggaaactttgttgtcacaatggccaaaagggaaattcgcattgggaatgctgtggtagagtacagtgggtccgagactgccgtagaaagaattaactcaacagatcgcattgagcaagaacttttgcttcaggttttgtcggtgggaaagttgtacaaccccgatgtacgctattctttcaatattccaattgaagataaacctcagcagttttactggaacagtcatgggccatggcaagcatgcagtaaaccctgccaaggggaacggaaacgaaaacttgtttgcaccagggaatctgatcagcttactgtttctgatcaaagatgcgatcggctgccccagcctggacacattactgaaccctgtggtacagactgtgacctgaggtggcatgttgccagcaggagtgaatgtagtgcccagtgtggcttgggttaccgcacattggacatctactgtgccaaatatagcaggctggatgggaagactgagaaggttgatgatggtttttgcagcagccatcccaaaccaagcaaccgtgaaaaatgctcaggggaatgtaacacgggtggctggcgctattctgcctggactgaatgttcaaaaagctgtgacggtgggacccagaggagaagggctatttgtgtcaatacccgaaatgatgtactggatgacagcaaatgcacacatcaagagaaagttaccattcagaggtgcagtgagttcccttgtccacagtggaaatctggagactggtcagagtgcttggtcacctgtggaaaagggcata

SEQ ID NO: 25 - DNA, 1948 bp, homo sapiens
atgcagtttgtatcctgggccacactgctaacgctcctggtgcgggacctggccgagatggggagcccagacgccgcggcggccgtgcgcaaggacaggctgcacccgaggcaagtgaaattattagagaccctgagcgaatacgaaatcgtgtctcccatccgagtgaacgctctcggagaaccctttcccacgaacgtccacttcaaaagaacgcgacggagcattaactctgccactgacccctggcctgccttcgcctcctcctcttcctcctctacctcctcccaggcgcattaccgcctctctgccttcggccagcagtttctatttaatctcaccgccaatgccggatttatcgctccactgttcactgtcaccctcctcgggacgcccggggtgaatcagaccaagttttattccgaagaggaagcggaactcaagcactgtttctacaaaggctatgtcaataccaactccgagcacacggccgtcatcagcctctgctcaggaatgctgggcacattccggtctcatgatggggattattttattgaaccactacagtctatggatgaacaagaagatgaagaggaacaaaacaaaccccacatcatttataggcgcagcgccccccagagagagccctcaacaggaaggcatgcatgtgacacctcagaacacaaaaataggcacagtaaagacaagaagaaaaccagagcaagaaaatggggagaaaggattaacctggctggtgacgtagcagcattaaacagcggcttagcaacagaggcattttctgcttatggtaataagacggacaacacaagagaaaagaggacccacagaaggacaaaacgttttttatcctatccacggtttgtagaagtcttggtggtggcagacaacagaatggtttcataccatggagaaaaccttcaacactatattttaactttaatgtcaattgtagcctctatctataaagacccaagtattggaaatttaattaatattgttattgtgaacttaattgtgattcataatgaacaggatgggccttccatatcttttaatgctcagacaacattaaaaaacttttgccagtggcagcattcgaagaacagtccaggtggaatccatcatgatactgctgttctcttaacaagacaggatatctgcagagctcacgacaaatgtgataccttaggcctggctgaactgggaaccatttgtgatccctatagaagctgttctattagtgaagatagtggattgagtacagcttttacgatcgcccatgagctgggccatgtgtttaacatgcctcatgatgacaacaacaaatgtaaagaagaaggagttaagagtccccagcatgtcatggctccaacactgaacttctacaccaacccctggatgtggtcaaagtgtagtcgaaaatatatcactgagtttttagacactggttatggcgagtgtttgcttaacgaacctgaatccagaccctaccctttgcctgtccaactgccaggcatcctttacaacgtgaataaacaatgtgaattgatttttggaccaggttctcaggtgtgcccatatatgatgcagtgcagacggctctggtgcaataacgtcaatggagtacacaaaggctgccggactcagcacacaccctgggccgatgggacggagtgcgagcctggaaagcactgcaagtatggattttgtgttcccaaagaaatggatgtccccgtgacagatggatcctggggaagttggagtccctttggaacctgctccagaacatgtggagggggcatcaaaacagccattcgagagtgcaacagaccagaaccaaaaaatggtggaaaatactgtgtaggacgtagaatgaaatttaagtcctgcaacacggagccatgtctcaagcagaagcgagacttcc

SEQ ID NO: 26 - DNA, 1084 bp, homo sapiens
ttatcctatccacggtttgtagaagtcttggtggtggcagacaacagaatggtttcataccatggagaaaaccttcaacactatattttaactttaatgtcaattgtagcctctatctataaagacccaagtattggaaatttaattaatattgttattgtgaacttaattgtgattcataatgaacaggatgggccttccatatcttttaatgctcagacaacattaaaaaacttttgccagtggcagcattcgaagaacagtccaggtggaatccatcatgatactgctgttctcttaacaagacaggatatctgcagagctcacgacaaatgtgataccttaggcctggctgaactgggaaccatttgtgatccctatagaagctgttctattagtgaagatagtggattgagtacagcttttacgatcgcccatgagctgggccatgtgtttaacatgcctcatgatgacaacaacaaatgtaaagaagaaggagttaagagtccccagcatgtcatggctccaacactgaacttctacaccaacccctggatgtggtcaaagtgtagtcgaaaatatatcactgagtttttagacactggttatggcgagtgtttgcttaacgaacctgaatccagaccctaccctttgcctgtccaactgccaggcatcctttacaacgtgaataaacaatgtgaattgatttttggaccaggttctcaggtgtgcccatatatgatgcagtgcagacggctctggtgcaataacgtcaatggagtacacaaaggctgccggactcagcacacaccctgggccgatgggacggagtgcgagcctggaaagcactgcaagtatggattttgtgttcccaaagaaatggatgtccccgtgacagatggatcctggggaagttggagtccctttggaacctgctccagaacatgtggagggggcatcaaaacagccattcgagagtgcaacagaccagaaccaaaaaatggtggaaaatactgtgtaggacgtagaatgaaatttaagtcctgcaacacggagccatgtctcaagcagaagcgagacttcc

SEQ ID NO: 27 - DNA, 2608 bp, homo sapiens
ggccatggtggctgcggaccttgaagccaccgcctgccaggcccctggggaatgaaacagagcgtggccagccaggcctgaagcgatcggtcagccgagagcgctacgtggagaccctggtggtggctgacaagatgatggtggcctatcacgggcgccgggatgtggagcagtatgtcctggccatcatgaacattgtcaggttgccaaacttttccaggactcgagtctgggaagcaccgttaacatcctcgtaactcgcctcatcctgctcacggaggaccagcccactctggagatcacccaccatgccgggaagtccctggacagcttctgtaagtggcagaaatccatcgtgaaccacagcggccatggcaatgccattccagagaacggtgtggctaaccatgacacagcagtgctcatcacacgctatgacatctgcatctacaagaacaaaccctgcggcacactaggcctggccccggtgggcggaatgtgtgagcgcgagagaagctgcagcgtcaatgaggacattggcctggccacagcgttcaccattgcccacgagatcgggcacacattcggcatgaaccatgacggcgtgggaaacagctgtggggcccgtggtcaggacccagccaagctcatggctgcccacattaccatgaagaccaacccattcgtgtggtcatcctgcagccgtgactacatcaccagctttctagactcgggcctggggctctgcctgaacaaccggccccccagacaggactttgtgtacccgacagtggcaccgggccaagcctacgatgcagatgagcaatgccgctttcagcatggagtcaaatcgcgtcagtgtaaatacggggaggtctgcagcgagctgtggtgtctgagcaagagcaaccggtgcatcaccaacagcatcccggccgccgagggcacgctgtgccagacgcacaccatcgacaaggggtggtgctacaaacgggtctgtgtcccctttgggtcgcgcccagagggtgtggacggagcctgggggccgtggactccatggggcgactgcagccggacctgtggcggcggcgtgtcctcttctagccgtcactgcgacagccccaggccaaccatcgggggcaagtactgtctgggtgagagaaggcggcaccgctcctgcaacacggatgactgtccccctggctcccaggacttcagagaagtgcagtgttctgaatttgacagcatccctttccgtgggaaattctacaagtggaaaacgtaccggggagggggcgtgaaggcctgctcgctcacgtgcctagcggaaggcttcaacttctacacggagagggcggcagccgtggtggacgggacaccctgccgtccagacacggtggacatttgcgtcagtggcgaatgcaagcacgtgggctgcgaccgagtcctgggctccgacctgcgggaggacaagtgccgagtgtgtggcggtgacggcagtgcctgcgagaccatcgagggcgtcttcagcccagcctcacctggggccgggtacgaggatgtcgtctggattcccaaaggctccgtccacatcttcatccaggatctgaacctctctctcagtcacttggccctgaagggagaccaggagtccctgctgctggaggggctgcccgggaccccccagccccaccgtctgcctctagctgggaccacctttcaactgcgacaggggccagaccaggtccagagcctcgaagccctgggaccgattaatgcatctctcatcgtcatggtgctggcccggaccgagctgcctgccctccgctaccgcttcaatgcccccatcgcccgtgactcgctgcccccctactcctggcactatgcgccctggaccaagtgctcggcccagtgtgcaggcggtagccaggtgcaggcggtggagtgccgcaaccagctggacagctccgcggtcgccccccactactgcagtgcccacagcaagctgcccaaaaggcagcgcgcctgcaacacggagccttgccctccagactgggttgtagggaactggtcgctctgcagccgcagctgcgatgcaggcgtgcgcagccgctcggtcgtgtgccagcgccgcgtctctgccgcggaggagaaggcgctggacgacagcgcatgcccgcagccgcgcccacctgtactggaggcctgccacggccccacttgccctccggagtgggcggccctcgactggtctgaggtcagccgcccccttccttcgcgcccactgggatgcccaggtggggtgtccacggagacgcctctgcgcggtctccaggttagctgtcccctcctcacttcgcactggggcagcaccctgagcacgaaacgccccttccaactgcagtgcacccccagctgcgggccgggcctccgccaccgcgtggtcctttgcaagagcgcagaccaccgcgccacgctgcccccggcgcactgctcacccgccgccaagccaccggccaccatgcgctgcaacttgcgccgctgccccccggcccgctgggtggctggcgagtggggtgagtgctctgcacagtgcggcgtcgggcag

SEQ ID NO: 28 - DNA, 1825 bp, homo sapiens
atgctgcgaaggctgtgaacaggggaggcggcactgtgggggctgccggcagccggggctggggagagacatgtggacacgtggcctctatggctcccgcctgccagatcctccgctgggccctcgccctggggctgggcctcatgttcgaggtcacgcacgccttccggtctcaagatgagttcctgtccagtctggagagctatgagatcgccttccccacccgcgtggaccacaacggggcactgctggccttctcgccacctcctccccggaggcagcgccgcggcacgggggccacagccgagtcccgcctcttctacaaagtggcctcgcccagcacccacttcctgctgaacctgacccgcagctcccgtctactggcagggcacgtctccgtggagtactggacacgggagggcctggcctggcagagggcggcccggccccactgcctctacgctggtcacctgcagggccaggccagcagctcccatgtggccatcagcacctgtggaggcctgcacggcctgatcgtggcagacgaggaagagtacctgattgagcccctgcacggtgggcccaagggttctcggagcccggaggaaagtggaccacatgtggtgtacaagcgttcctctctgcgtcacccccacctggacacagcctgtggagtgagagatgagaaaccgtggaaagggcggccatggtggctgcggaccttgaagccaccgcctgccaggcccctggggaatgaaacagagcgtggccagccaggcctgaagcgatcggtcagccgagagcgctacgtggagaccctggtggtggctgacaagatgatggtggcctatcacgggcgccgggatgtggagcagtatgtcctggccatcatgaacattgtcaggttgccaaacttttccaggactcgagtctgggaagcaccgttaacatcctcgtaactcgcctcatcctgctcacggaggaccagcccactctggagatcacccaccatgccgggaagtccctggacagcttctgtaagtggcagaaatccatcgtgaaccacagcggccatggcaatgccattccagagaacggtgtggctaaccatgacacagcagtgctcatcacacgctatgacatctgcatctacaagaacaaaccctgcggcacactaggcctggccccggtgggcggaatgtgtgagcgcgagagaagctgcagcgtcaatgaggacattggcctggccacagcgttcaccattgcccacgagatcgggcacacattcggcatgaaccatgacggcgtgggaaacagctgtggggcccgtggtcaggacccagccaagctcatggctgcccacattaccatgaagaccaacccattcgtgtggtcatcctgcagccgtgactacatcaccagctttctagactcgggcctggggctctgcctgaacaaccggccccccagacaggactttgtgtacccgacagtggcaccgggccaagcctacgatgcagatgagcaatgccgctttcagcatggagtcaaatcgcgtcagtgtaaatacggggaggtctgcagcgagctgtggtgtctgagcaagagcaaccggtgcatcaccaacagcatcccggccgccgagggcacgctgtgccagacgcacaccatcgacaaggggtggtgctacaaacgggtctgtgtcccctttgggtcgcgcccagagggtgtggacggagcctgggggccgtggactccatggggcgactgcagccggacctgtggcggcggcgtgtcctcttctagccgtcactgcgacagcccc

SEQ ID NO: 29 - DNA, 1130 bp, homo sapiens
ggccatggtggctgcggaccttgaagccaccgcctgccaggcccctggggaatgaaacagagcgtggccagccaggcctgaagcgatcggtcagccgagagcgctacgtggagaccctggtggtggctgacaagatgatggtggcctatcacgggcgccgggatgtggagcagtatgtcctggccatcatgaacattgtcaggttgccaaacttttccaggactcgagtctgggaagcaccgttaacatcctcgtaactcgcctcatcctgctcacggaggaccagcccactctggagatcacccaccatgccgggaagtccctggacagcttctgtaagtggcagaaatccatcgtgaaccacagcggccatggcaatgccattccagagaacggtgtggctaaccatgacacagcagtgctcatcacacgctatgacatctgcatctacaagaacaaaccctgcggcacactaggcctggccccggtgggcggaatgtgtgagcgcgagagaagctgcagcgtcaatgaggacattggcctggccacagcgttcaccattgcccacgagatcgggcacacattcggcatgaaccatgacggcgtgggaaacagctgtggggcccgtggtcaggacccagccaagctcatggctgcccacattaccatgaagaccaacccattcgtgtggtcatcctgcagccgtgactacatcaccagctttctagactcgggcctggggctctgcctgaacaaccggccccccagacaggactttgtgtacccgacagtggcaccgggccaagcctacgatgcagatgagcaatgccgctttcagcatggagtcaaatcgcgtcagtgtaaatacggggaggtctgcagcgagctgtggtgtctgagcaagagcaaccggtgcatcaccaacagcatcccggccgccgagggcacgctgtgccagacgcacaccatcgacaaggggtggtgctacaaacgggtctgtgtcccctttgggtcgcgcccagagggtgtggacggagcctgggggccgtggactccatggggcgactgcagccggacctgtggcggcggcgtgtcctcttctagccgtcactgcgacagccccaggccaa

SEQ ID NO: 30 - DNA, 2380 bp, homo sapiens
ctctcttctgaggtcccatagaaatgaagaactgaacgtggagaccttggtggtggtcgacaaaaagatgatgcaaaaccatggccatgaaaatatcaccacctacgtgctcacgatactcaacatggtatctgctttattcaaagatggaacaataggaggaaacatcaacattgcaattgtaggtctgattcttctagaagatgaacagccaggactggtgataagtcaccacgcagaccacaccttaagtagcttctgccagtggcagtctggattgatggggaaagatgggactcgtcatgaccacgccatcttactgactggtctggatatatgttcctggaagaatgagccctgtgacactttgggatttgcacccataagtggaatgtgtagtaaatatcgcagctgcacgattaatgaagatacaggtcttggactggccttcaccattgcccatgagtctggacacaactttggcatgattcatgatggagaagggaacatgtgtaaaaagtccgagggcaacatcatgtcccctacattggcaggacgcaatggagtcttctcctggtcaccctgcagccgccagtatctacacaaatttctaagcaccgctcaagctatctgccttgctgatcagccaaagcctgtgaaggaatacaagtatcctgagaaattgccaggagaattatatgatgcaaacacacagtgcaagtggcagttcggagagaaagccaagctctgcatgctggactttaaaaaggacatctgtaaagccctgtggtgccatcgtattggaaggaaatgtgagactaaatttatgccagcagcagaaggcacaatttgtgggcatgacatgtggtgccggggaggacagtgtgtgaaatatggtgatgaaggccccaagcccacccatggccactggtcggactggtcttcttggtccccatgctccaggacctgcggagggggagtatctcataggagtcgcctctgcaccaaccccaagccatcgcatggagggaagttctgtgagggctccactcgcactctgaagctctgcaacagtcagaaatgtccccgggacagtgttgacttccgtgctgctcagtgtgccgagcacaacagcagacgattcagagggcggcactacaagtggaagccttacactcaagtagaagatcaggacttatgcaaactctactgtatcgcagaaggatttgatttcttcttttctttgtcaaataaagtcaaagatgggactccatgctcggaggatagccgtaatgtttgtatagatgggatatgtgagagagttggatgtgacaatgtccttggatctgatgctgttgaagacgtctgtggggtgtgtaacgggaataactcagcctgcacgattcacaggggtctctacaccaagcaccaccacaccaaccagtattatcacatggtcaccattccttctggagcccggagtatccgcatctatgaaatgaacgtctctacctcctacatttctgtgcgcaatgccctcagaaggtactacctgaatgggcactggaccgtggactggcccggccggtacaaattttcgggcactactttcgactacagacggtcctataatgagcccgagaacttaatcgctactggaccaaccaacgagacactgattgtggagctgctgtttcagggaaggaacccgggtgttgcctgggaatactccatgcctcgcttggggaccgagaagcagccccctgcccagcccagctacacttgggccatcgtgcgctctgagtgctccgtgtcctgcggagggggacagatgaccgtgagagagggctgctacagagacctgaagtttcaagtaaatatgtccttctgcaatcccaagacacgacctgtcacggggctggtgccttgcaaagtatctgcctgtcctcccagctggtccgtggggaactggagtgcctgcagtcggacgtgtggcgggggtgcccagagccgccccgtgcagtgcacacggcgggtgcactatgactcggagccagtcccggccagcctgtgccctcagcctgctccctccagcaggcaggcctgcaactctcagagctgcccacctgcatggagcgccgggccctgggcagagtgctcacacacctgtgggaaggggtggaggaagcgggcagtggcctgtaagagcaccaacccctcggccagagcgcagctgctgcccgacgctgtctgcacctccgagcccaagcccaggatgcatgaagcctgtctgcttcagcgctgccacaagcccaagaagctgcagtggctggtgtccgcctggtcccaggtaggtgcactggtctcgcgggagcgaggt

SEQ ID NO: 31 - DNA, 1941 bp, homo sapiens
atgaagccccgcgcgcgcggatggcggggcttggcggcgctgtggatgctgctggcgcaggtggccgagcaggcacctgcgtgcgccatgggacccgcagcggcagcgcctgggagcccgagcgtcccgcgtcctcctccacccgcggagcggccgggctggatggaaaagggcgaatatgacctggtctctgcctacgaggttgaccacaggggcgattacgtgtcccatgaaatcatgcaccatcagcggcggagaagagcagtggccgtgtccgaggttgagtctcttcaccttcggctgaaaggccccaggcacgacttccacatggatctgaggacttccagcagcctagtggctcctggctttattgtgcagacgttgggaaagacaggcactaagtctgtgcagactttaccgccagaggacttctgtttctatcaaggctctttgcgatcacacagaaactcctcagtggccctttcaacctgccaaggcttgtcaggcatgatacgaacagaagaggcagattacttcctaaggccacttccttcacacctctcatggaaactcggcagagctgcccaaggcagctcgccatcccacgtactgtacaagagatccacagagccccatgctcctggggccagtgaggtcctggtgacctcaaggacatgggagctggcacatcaacccctgcacagcagcgaccttcgcctgggactgccacaaaagcagcatttctgtggaagacgcaagaaatacatgccccagcctcccaaggaagacctcttcatcttgccagatgagtataagtcttgcttacggcataagcgctctcttctgaggtcccatagaaatgaagaactgaacgtggagaccttggtggtggtcgacaaaaagatgatgcaaaaccatggccatgaaaatatcaccacctacgtgctcacgatactcaacatggtatctgctttattcaaagatggaacaataggaggaaacatcaacattgcaattgtaggtctgattcttctagaagatgaacagccaggactggtgataagtcaccacgcagaccacaccttaagtagcttctgccagtggcagtctggattgatggggaaagatgggactcgtcatgaccacgccatcttactgactggtctggatatatgttcctggaagaatgagccctgtgacactttgggatttgcacccataagtggaatgtgtagtaaatatcgcagctgcacgattaatgaagatacaggtcttggactggccttcaccattgcccatgagtctggacacaactttggcatgattcatgatggagaagggaacatgtgtaaaaagtccgagggcaacatcatgtcccctacattggcaggacgcaatggagtcttctcctggtcaccctgcagccgccagtatctacacaaatttctaagcaccgctcaagctatctgccttgctgatcagccaaagcctgtgaaggaatacaagtatcctgagaaattgccaggagaattatatgatgcaaacacacagtgcaagtggcagttcggagagaaagccaagctctgcatgctggactttaaaaaggacatctgtaaagccctgtggtgccatcgtattggaaggaaatgtgagactaaatttatgccagcagcagaaggcacaatttgtgggcatgacatgtggtgccggggaggacagtgtgtgaaatatggtgatgaaggccccaagcccacccatggccactggtcggactggtcttcttggtccccatgctccaggacctgcggagggggagtatctcataggagtcgcctctgcaccaaccccaagccatcgcatggagggaagttctgtgagggctccactcgcactctgaagctctgcaacagtcagaaatgtccccgggacagtgttgacttc

SEQ ID NO: 32 - DNA, 1105 bp, homo sapiens
ctctcttctgaggtcccatagaaatgaagaactgaacgtggagaccttggtggtggtcgacaaaaagatgatgcaaaaccatggccatgaaaatatcaccacctacgtgctcacgatactcaacatggtatctgctttattcaaagatggaacaataggaggaaacatcaacattgcaattgtaggtctgattcttctagaagatgaacagccaggactggtgataagtcaccacgcagaccacaccttaagtagcttctgccagtggcagtctggattgatggggaaagatgggactcgtcatgaccacgccatcttactgactggtctggatatatgttcctggaagaatgagccctgtgacactttgggatttgcacccataagtggaatgtgtagtaaatatcgcagctgcacgattaatgaagatacaggtcttggactggccttcaccattgcccatgagtctggacacaactttggcatgattcatgatggagaagggaacatgtgtaaaaagtccgagggcaacatcatgtcccctacattggcaggacgcaatggagtcttctcctggtcaccctgcagccgccagtatctacacaaatttctaagcaccgctcaagctatctgccttgctgatcagccaaagcctgtgaaggaatacaagtatcctgagaaattgccaggagaattatatgatgcaaacacacagtgcaagtggcagttcggagagaaagccaagctctgcatgctggactttaaaaaggacatctgtaaagccctgtggtgccatcgtattggaaggaaatgtgagactaaatttatgccagcagcagaaggcacaatttgtgggcatgacatgtggtgccggggaggacagtgtgtgaaatatggtgatgaaggccccaagcccacccatggccactggtcggactggtcttcttggtccccatgctccaggacctgcggagggggagtatctcataggagtcgcctctgcaccaaccccaagccatcgcatggagggaagttctgtgagggctccactcgcactctgaagctctgcaacagtcagaaatgtccccgggacagtgttgacttc

SEQ ID NO: 33 - DNA, 2799 bp, homo sapiens
accatcgaaggttgcaaaagcagcatttttgtggacgacgcaagaaatatgctcccaagcctcccacagaggacacctatctaaggtttgatgaatatgggagctctgggcgacccagaagatcagctggaaaatcacaaaagggcctcaatgtggaaaccctcgtggtggcagacaagaaaatggtggaaaagcatggcaagggaaatgtcaccacatacattctcacagtaatgaacatggtttctggcctatttaaagatgggactattggaagtgacataaacgtggttgtggtgagcctaattcttctggaacaagaacctggaggattattgatcaaccatcatgcagaccagtctctgaatagtttttgtcaatggcagtctgccctcattggaaagaatggcaagagacatgatcatgccatcttactaacaggatttgatatttgttcttggaagaatgaaccatgtgacactctagggtttgcccccatcagtggaatgtgctctaagtaccgaagttgtaccatcaatgaggacacaggacttggccttgccttcaccatcgctcatgagtcagggcacaactttggtatgattcacgatggagaagggaatccctgcagaaaggctgaaggcaatatcatgtctcccacactgaccggaaacaatggagtgttttcatggtcttcctgcagccgccagtatctcaagaaattcctcagcacacctcaggcggggtgtctagtggatgagcccaagcaagcaggacagtataaatatccggacaaactaccaggacagatttatgatgctgacacacagtgtaaatggcaatttggagcaaaagccaagttatgcagccttggttttgtgaaggatatttgcaaatcactttggtgccaccgagtaggccacaggtgtgagaccaagtttatgcccgcagcagaagggaccgtttgtggcttgagtatgtggtgtcggcaaggccagtgcgtaaagtttggggagctcgggccccggcccatccacggccagtggtccgcctggtcgaagtggtcagaatgttcccggacatgtggtggaggagtcaagttccaggagagacactgcaataaccccaagcctcagtatggtggcttattctgtccaggttctagccgtatttatcagctgtgcaatattaacccttgcaatgaaaatagcttggattttcgggctcaacagtgtgcagaatataacagcaaacctttccgtggatggttctaccagtggaaaccctatacaaaagtggaagaggaagatcgatgcaaactgtactgcaaggctgagaactttgaatttttttttgcaatgtccggcaaagtgaaagatggaactccctgctccccaaacaaaaatgatgtttgtattgacggggtttgtgaactagtgggatgtgatcatgaactaggctctaaagcagtttcagatgcttgtggcgtttgcaaaggtgataattcaacttgcaagttttataaaggcctgtacctcaaccagcataaagcaaatgaatattatccggtggtcctcattccagctggcgcccgaagcatcgaaatccaggagctgcaggtttcctccagttacctcgcagttcgaagcctcagtcaaaagtattacctcaccgggggctggagcatcgactggcctggggagttccccttcgctgggaccacgtttgaataccagcgctctttcaaccgcccggaacgtctgtacgcgccagggcccacaaatgagacgctggtctttgaaattctgatgcaaggcaaaaatccagggatagcttggaagtatgcacttcccaaggtcatgaatggaactccaccagccacaaaaagacctgcctatacctggagtatcgtgcagtcagagtgctccgtctcctgtggtggaggttacataaatgtaaaggccatttgcttgcgagatcaaaatactcaagtcaattcctcattctgcagtgcaaaaaccaagccagtaactgagcccaaaatctgcaacgctttctcctgcccggcttactggatgccaggtgaatggagtacatgcagcaaggcctgtgctggaggccagcagagccgaaagatccagtgtgtgcaaaagaagcccttccaaaaggaggaagcagtgttgcattctctctgtccagtgagcacacccactcaggtccaagcctgcaacagccatgcctgccctccacaatggagccttggaccctggtctcagtgttccaagacctgtggacgaggggtgaggaagcgtgaactcctctgcaagggctctgccgcagaaaccctccccgagagccagtgtaccagtctccccagacctgagctgcaggagggctgtgtgcttggacgatgccccaagaacagccggctacagtgggtcgcttcttcgtggagcgagtgttctgcaacctgtggtttgggtgtgaggaagagggagatgaagtgcagcgagaagggcttccagggaaagctgataactttcccagagcgaagatgccgtaatattaagaaaccaaatctggacttggaagagacctgcaaccgacgggcttgcccagcccatccagtgtacaacatggtagctggatggtattcattgccgtggcagcagtgcacagtcacctgtgggggaggggtccagacccggtcagtccactgtgttcagcaaggccggccttcctcaagttgtctgctccatcagaaacctccggtgctacgagcctgtaatacaaacttct

SEQ ID NO: 34 - DNA, 1951 bp, homo sapiens
atgtgaacggcggcggctctcacctggagccgcacctggggcgccgagctccggggccgcggaaagaatgcgcgccgcccgtgcgctccgcctgccgcgtctggccacccgcagccgccgcgtccgcacctgaccatggagtgcgccctcctgctcgcgtgtgccttcccggctgcgggttcgggcccgccgaggggcctggcgggactggggcgcgtggccaaggcgctccagctgtgctgcctctgctgtgcgtcggtcgccgcggccttagccagtgacagcagcagcggcgccagcggattaaatgatgattacgtctttgtcacgccagtagaagtagactcagccgggtcatatatttcacacgacattttgcacaacggcaggaaaaagcgatcggcgcagaatgccagaagctccctgcactaccgattttcagcatttggacaggaactgcacttagaacttaagccctcggcgattttgagcagtcactttattgtccaggtacttggaaaagatggtgcttcagagactcagaaacccgaggtgcagcaatgcttctatcagggatttatcagaaatgacagctcctcctctgtcgctgtgtctacgtgtgctggcttgtcaggtttaataaggacacgaaaaaatgaattcctcatctcgccattacctcagcttctggcccaggaacacaactacagctcccctgcgggtcaccatcctcacgtactgtacaaaaggacagcagaggagaagatccagcggtaccgtggctaccccggctctggccggaattatcctggttactccccaagtcacattccccatgcatctcagagtcgagagacagagtatcaccatcgaaggttgcaaaagcagcatttttgtggacgacgcaagaaatatgctcccaagcctcccacagaggacacctatctaaggtttgatgaatatgggagctctgggcgacccagaagatcagctggaaaatcacaaaagggcctcaatgtggaaaccctcgtggtggcagacaagaaaatggtggaaaagcatggcaagggaaatgtcaccacatacattctcacagtaatgaacatggtttctggcctatttaaagatgggactattggaagtgacataaacgtggttgtggtgagcctaattcttctggaacaagaacctggaggattattgatcaaccatcatgcagaccagtctctgaatagtttttgtcaatggcagtctgccctcattggaaagaatggcaagagacatgatcatgccatcttactaacaggatttgatatttgttcttggaagaatgaaccatgtgacactctagggtttgcccccatcagtggaatgtgctctaagtaccgaagttgtaccatcaatgaggacacaggacttggccttgccttcaccatcgctcatgagtcagggcacaactttggtatgattcacgatggagaagggaatccctgcagaaaggctgaaggcaatatcatgtctcccacactgaccggaaacaatggagtgttttcatggtcttcctgcagccgccagtatctcaagaaattcctcagcacacctcaggcggggtgtctagtggatgagcccaagcaagcaggacagtataaatatccggacaaactaccaggacagatttatgatgctgacacacagtgtaaatggcaatttggagcaaaagccaagttatgcagccttggttttgtgaaggatatttgcaaatcactttggtgccaccgagtaggccacaggtgtgagaccaagtttatgcccgcagcagaagggaccgtttgtggcttgagtatgtggtgtcggcaaggccagtgcgtaaagtttggggagctcgggccccggcccatccacggccagtggtccgcctggtcgaagtggtcagaatgttcccggacatgtg

SEQ ID NO: 35 - DNA, 1086 bp, homo sapiens
accatcgaaggttgcaaaagcagcatttttgtggacgacgcaagaaatatgctcccaagcctcccacagaggacacctatctaaggtttgatgaatatgggagctctgggcgacccagaagatcagctggaaaatcacaaaagggcctcaatgtggaaaccctcgtggtggcagacaagaaaatggtggaaaagcatggcaagggaaatgtcaccacatacattctcacagtaatgaacatggtttctggcctatttaaagatgggactattggaagtgacataaacgtggttgtggtgagcctaattcttctggaacaagaacctggaggattattgatcaaccatcatgcagaccagtctctgaatagtttttgtcaatggcagtctgccctcattggaaagaatggcaagagacatgatcatgccatcttactaacaggatttgatatttgttcttggaagaatgaaccatgtgacactctagggtttgcccccatcagtggaatgtgctctaagtaccgaagttgtaccatcaatgaggacacaggacttggccttgccttcaccatcgctcatgagtcagggcacaactttggtatgattcacgatggagaagggaatccctgcagaaaggctgaaggcaatatcatgtctcccacactgaccggaaacaatggagtgttttcatggtcttcctgcagccgccagtatctcaagaaattcctcagcacacctcaggcggggtgtctagtggatgagcccaagcaagcaggacagtataaatatccggacaaactaccaggacagatttatgatgctgacacacagtgtaaatggcaatttggagcaaaagccaagttatgcagccttggttttgtgaaggatatttgcaaatcactttggtgccaccgagtaggccacaggtgtgagaccaagtttatgcccgcagcagaagggaccgtttgtggcttgagtatgtggtgtcggcaaggccagtgcgtaaagtttggggagctcgggccccggcccatccacggccagtggtccgcctggtcgaagtggtcagaatgttcccggacatgtg

SEQ ID NO: 36 - protein, 8 aa, artificial
  strep-tag
WSHPQFEK

SEQ ID NO: 37 - DNA, 27 bp, artificial
  Strep-tag
tggagccacccgcagttcgaaaaataa

SEQ ID NO: 38 - protein, 11 aa, artificial
  peptide linker
GSAWSHPQFEK

SEQ ID NO: 39 - DNA, 36 bp, artificial
  peptide linker
ggaagcgcttggagccacccgcagttcgaaaaataa

SEQ ID NO: 40 - protein, 375 aa, homo sapiens
MHQRSVSKEKWVETLVVADAKMVEYHGQPQVESYVLTIMNMVAGLFHDPSIGNPIHITIVRLVLLEDEEEDLKITHHADNTLKSFCKWQKSINMKGDAHPLHHDTAILLTRKDLCAAMNRPCETLGLSHVAGMCQPHRSCSINEDTGLPLAFTVAHELGHSFGIQHDGSGNDCEPVGKRPFIMSPQLLYDAAPLTWSRCSRQYITRFLDRGWGLCLDDPPAKDIIDFPSVPPGVLYDVSHQCRLQYGAYSAFCEDMDNVCHTLWCSVGTTCHSKLDAAVDGTRCGENKWCLSGECVPVGFRPEAVDGGWSGWSAWSICSRSCGMGVQSAERQCTQPTPKYKGRYCVGERKRFRLCNLQACPAGRPSFWSHPQFEK

SEQ ID NO: 41 - protein, 370 aa, homo sapiens
MLSYPRFVEVLVVADNRMVSYHGENLQHYILTLMSIVASIYKDPSIGNLINIVIVNLIVIHNEQDGPSISFNAQTTLKNLCQWQHSKNSPGGIHHDTAVLLTRQDICRAHDKCDTLGLAELGTICDPYRSCSISEDSGLSTAFTIAHELGHVFNMPHDDNNKCKEEGVKSPQHVMAPTLNFYTNPWMWSKCSRKYITEFLDTGYGECLLNEPESRPYPLPVQLPGILYNVNKQCELIFGPGSQVCPYMMQCRRLWCNNVNGVHKGCRTQHTPWADGTECEPGKHCKYGFCVPKEMDVPVTDGSWGSWSPFGTCSRTCGGGIKTAIRECNRPEPKNGGKYCVGRRMKFKSCNTEPCLKQKRDFWSHPQFEK

SEQ ID NO: 42 - protein, 619 aa, homo sapiens
MAPACQILRWALALGLGLMFEVTHAFRSQDEFLSSLESYEIAFPTRVDHNGALLAFSPPPPRRQRRGTGATAESRLFYKVASPSTHFLLNLTRSSRLLAGHVSVEYWTREGLAWQRAARPHCLYAGHLQGQASSSHVAISTCGGLHGLIVADEEEYLIEPLHGGPKGSRSPEESGPHVVYKRSSLRHPHLDTACGVRDEKPWKGRPWWLRTLKPPPARPLGNETERGQPGLKRSVSRERYVETLVVADKMMVAYHGRRDVEQYVLAIMNIVAKLFQDSSLGSTVNILVTRLILLTEDQPTLEITHHAGKSLDSFCKWQKSIVNHSGHGNAIPENGVANHDTAVLITRYDICIYKNKPCGTLGLAPVGGMCERERSCSVNEDIGLATAFTIAHEIGHTFGMNHDGVGNSCGARGQDPAKLMAAHITMKTNPFVWSSCSRDYITSFLDSGLGLCLNNRPPRQDFVYPTVAPGQAYDADEQCRFQHGVKSRQCKYGEVCSELWCLSKSNRCITNSIPAAEGTLCQTHTIDKGWCYKRVCVPFGSRPEGVDGAWGPWTPWGDCSRTCGGGVSSSSRHCDSPRPTIGGKYCLGERRRHRSCNTDDCPPGSQDFGSAWSHPQFEK

SEQ ID NO: 43 - protein, 658 aa, homo sapiens
MKPRARGWRGLAALWMLLAQVAEQAPACAMGPAAAAPGSPSVPRPPPPAERPGWMEKGEYDLVSAYEVDHRGDYVSHEIMHHQRRRRAVAVSEVESLHLRLKGPRHDFHMDLRTSSSLVAPGFIVQTLGKTGTKSVQTLPPEDFCFYQGSLRSHRNSSVALSTCQGLSGMIRTEEADYFLRPLPSHLSWKLGRAAQGSSPSHVLYKRSTEPHAPGASEVLVTSRTWELAHQPLHSSDLRLGLPQKQHFCGRRKKYMPQPPKEDLFILPDEYKSCLRHKRSLLRSHRNEELNVETLVVVDKKMMQNHGHENITTYVLTILNMVSALFKDGTIGGNINIAIVGLILLEDEQPGLVISHHADHTLSSFCQWQSGLMGKDGTRHDHAILLTGLDICSWKNEPCDTLGFAPISGMCSKYRSCTINEDTGLGLAFTIAHESGHNFGMIHDGEGNMCKKSEGNIMSPTLAGRNGVFSWSPCSRQYLHKFLSTAQAICLADQPKPVKEYKYPEKLPGELYDANTQCKWQFGEKAKLCMLDFKKDICKALWCHRIGRKCETKFMPAAEGTICGHDMWCRGGQCVKYGDEGPKPTHGHWSDWSSWSPCSRTCGGGVSHRSRLCTNPKPSHGGKFCEGSTRTLKLCNSQKCPRDSVDFGSAWSHPQFEK

SEQ ID NO: 44 - protein, 661 aa, homo sapiens
MECALLLACAFPAAGSGPPRGLAGLGRVAKALQLCCLCCASVAAALASDSSSGASGLNDDYVFVTPVEVDSAGSYISHDILHNGRKKRSAQNARSSLHYRFSAFGQELHLELKPSAILSSHFIVQVLGKDGASETQKPEVQQCFYQGFIRNDSSSSVAVSTCAGLSGLIRTRKNEFLISPLPQLLAQEHNHSSPAGHHPHVLYKRTAEEKIQRYRGYPGSGRNYPGYSPSHIPHASQSRETEYHHRRLQKQHFCGRRKKYAPKPPTEDTYLRFDEYGSSGRPRRSAGKSQKGLNVETLVVADKKMVEKHGKGNVTTYILTVMNMVSGLFKDGTIGSDINVVVVSLILLEQEPGGLLINHHADQSLNSFCQWQSALIGKNGKRHDHAILLTGFDICSWKNEPCDTLGFAPISGMCSKYRSCTINEDTGLGLAFTIAHESGHNFGMIHDGEGNPCRKAEGNIMSPTLTGNNGVFSWSSCSRQYLKKFLSTPQAGCLVDEPKQAGQYKYPDKLPGQIYDADTQCKWQFGAKAKLCSLGFVKDICKSLWCHRVGHRCETKFMPAAEGTVCGLSMWCRQGQCVKFGELGPRPIHGQWSAWSKWSECSRTCGGGVKFQERHCNNPKPQYGGIFCPGSSRIYQLCNINPCNENSLDFGSAWSHPQFEK